SEQUENCE LISTING (41 sequences)

SEQ ID NO: 1 - protein, 501 aa, Homo sapiens
MEQQDQSMKEGRLTLVLALATLIAAFGSSFQYGYNVAAVNSPALLMQQFYNETYYGRTGEFMEDFPLTLLWSVTVSMFPFGGFIGSLLVGPLVNKFGRKGALLFNNIFSIVPAILMGCSRVATSFELIIISRLLVGICAGVSSNVVPMYLGELAPKNLRGALGVVPQLFITVGILVAQIFGLRNLLANVDGWPILLGLTGVPAALQLLLLPFFPESPRYLLIQKKDEAAAKKALQTLRGWDSVDREVAEIRQEDEAEKAAGFISVLKLFRMRSLRWQLLSIIVLMGGQQLSGVNAIYYYADQIYLSAGVPEEHVQYVTAGTGAVNVVMTFCAVFVVELLGRRLLLLLGFSICLIACCVLTAALALQDTVSWMPYISIVCVISYVIGHALGPSPIPALLITEIFLQSSRPSAFMVGGSVHWLSNFTVGLIFPFIQEGLGPYSFIVFAVICLLTTIYIFLIVPETKAKTFIEINQIFTKMNKVSEVYPEKEELKELPPVTSEQ

SEQ ID NO: 2 - protein, 244 aa, Homo sapiens
MEQQDQSMKEGRLTLVLALATLIAAFGSSFQYGYNVAAVNSPALLMQQFYNETYYGRTGEFMEDFPLTLLWSVTVSMFPFGGFIGSLLVGPLVNKFGRKGALLFNNIFSIVPAILMGCSRVATSFELIIISRLLVGICAGVSSNVVPMYLGELAPKNLRGALGVVPQLFITVGILVAQIFGLRNLLANVDGEFRTSREHPHPFTTTLGPLLVFQSHHHRTGLSADWSLLTGWMSLGGPSCPEPT

SEQ ID NO: 3 - protein, 15 aa, synthetic construct
GGGGSGGGGSGGGGS

SEQ ID NO: 4 - DNA, 45 bp, synthetic construct
  source 1..45: mol_type = other DNA
ggcggcggcggatctggaggtggtggctcaggtggcggaggctcc

SEQ ID NO: 5 - protein, 8 aa, synthetic construct
  VARIANT 2: note = Valine or Isoleucine
  VARIANT 4: note = Any amino acid
DXEXNPGP

SEQ ID NO: 6 - protein, 75 aa, synthetic construct
  VARIANT 1..75: note = This sequence may encompass 1-15 GGGGS repeating units
GGGGSGGGGSGGGGSGGGGSGGGGSGGGGSGGGGSGGGGSGGGGSGGGGSGGGGSGGGGSGGGGSGGGGSGGGGS

SEQ ID NO: 7 - DNA, 735 bp, Homo sapiens
  source 1..735: mol_type = genomic DNA
atggagcaacaggatcagagcatgaaggaagggaggctgacgcttgtgcttgccctggcaaccctgatagctgcctttgggtcatccttccagtatgggtacaacgtggctgctgtcaactccccagcactgctcatgcaacaattttacaatgagacttactatggtaggaccggtgaattcatggaagacttccccttgacgttgctgtggtctgtaaccgtgtccatgtttccatttggagggtttatcggatccctcctggtcggccccttggtgaataaatttggcagaaaaggggccttgctgttcaacaacatattttctatcgtgcctgcgatcttaatgggatgcagcagagtcgccacatcatttgagcttatcattatttccagacttttggtgggaatatgtgcaggtgtatcttccaacgtggtccccatgtacttaggggagctggcccctaaaaacctgcggggggctctcggggtggtgccccagctcttcatcactgttggcatccttgtggcccagatctttggtcttcggaatctccttgcaaacgtagatggtgagttcaggacatctcgggagcacccccaccccttcaccactacccttggccccctccttgtgttccaaagccaccaccacaggacaggactttctgcagactggtctcttctaacaggctggatgtccttggggggcccatcctgtcccgagccaacatag

SEQ ID NO: 8 - DNA, 1506 bp, Homo sapiens
  source 1..1506: mol_type = genomic DNA
atggagcaacaggatcagagcatgaaggaagggaggctgacgcttgtgcttgccctggcaaccctgatagctgcctttgggtcatccttccagtatgggtacaacgtggctgctgtcaactccccagcactgctcatgcaacaattttacaatgagacttactatggtaggaccggtgaattcatggaagacttccccttgacgttgctgtggtctgtaaccgtgtccatgtttccatttggagggtttatcggatccctcctggtcggccccttggtgaataaatttggcagaaaaggggccttgctgttcaacaacatattttctatcgtgcctgcgatcttaatgggatgcagcagagtcgccacatcatttgagcttatcattatttccagacttttggtgggaatatgtgcaggtgtatcttccaacgtggtccccatgtacttaggggagctggcccctaaaaacctgcggggggctctcggggtggtgccccagctcttcatcactgttggcatccttgtggcccagatctttggtcttcggaatctccttgcaaacgtagatggctggccgatcctgctggggctgaccggggtccccgcggcgctgcagctccttctgctgcccttcttccccgagagccccaggtacctgctgattcagaagaaagacgaagcggccgccaagaaagccctacagacgctgcgcggctgggactctgtggacagggaggtggccgagatccggcaggaggatgaggcagagaaggccgcgggcttcatctccgtgctgaagctgttccggatgcgctcgctgcgctggcagctgctgtccatcatcgtcctcatgggcggccagcagctgtcgggcgtcaacgctatctactactacgcggaccagatctacctgagcgccggcgtgccggaggagcacgtgcagtacgtgacggccggcaccggggccgtgaacgtggtcatgaccttctgcgccgtgttcgtggtggagctcctgggtcggaggctgctgctgctgctgggcttctccatctgcctcatagcctgctgcgtgctcactgcagctctggcactgcaggacacagtgtcctggatgccatacatcagcatcgtctgtgtcatctcctacgtcataggacatgccctcgggcccagtcccatacccgcgctgctcatcactgagatcttcctgcagtcctctcggccatctgccttcatggtggggggcagtgtgcactggctctccaacttcaccgtgggcttgatcttcccgttcatccaggagggcctcggcccgtacagcttcattgtcttcgccgtgatctgcctcctcaccaccatctacatcttcttgattgtcccggagaccaaggccaagacgttcatagagatcaaccagattttcaccaagatgaataaggtgtctgaagtgtacccggaaaaggaggaactgaaagagcttccacctgtcacttcggaacagtga

SEQ ID NO: 9 - DNA, 1506 bp, Homo sapiens; identical to SEQ ID NO: 8
  source 1..1506: mol_type = genomic DNA

SEQ ID NO: 10 - protein, 21 aa, Homo sapiens
MALPVTALLLPLALLLHAARP

SEQ ID NO: 11 - DNA, 63 bp, Homo sapiens
  source 1..63: mol_type = genomic DNA
atggccctgccagtaacggctctgctgctgccacttgctctgctcctccatgcagccaggcct

SEQ ID NO: 12 - protein, 18 aa, Homo sapiens
MALPVTALLLPLALLLHA

SEQ ID NO: 13 - DNA, 54 bp, Homo sapiens
  source 1..54: mol_type = genomic DNA
atggctctcccagtgactgccctactgcttcccctagcgcttctcctgcatgca

SEQ ID NO: 14 - protein, 21 aa, Mus sp.
MASPLTRFLSLNLLLLGESII

SEQ ID NO: 15 - DNA, 63 bp, Mus sp.
  source 1..63: mol_type = genomic DNA
atggccagccccctgaccaggttcctgagcctgaacctgctgctgctgggcgagagcatcatc

SEQ ID NO: 16 - protein, 18 aa, Mus sp.
MASPLTRFLSLNLLLLGE

SEQ ID NO: 17 - DNA, 54 bp, Mus sp.
  source 1..54: mol_type = genomic DNA
atggccagccccctgaccaggttcctgagcctgaacctgctgctgctgggcgag

SEQ ID NO: 18 - protein, 214 aa, synthetic construct
MLRLLLALNLFPSIQVTGNKILVKQSPMLVAYDNALSCKYSYNLFSREFRASLHKGLDSAVEVCWYGNYSQQLQVYSKTGFNCDGKLGNESVTFYLQNLYQTDIYFCKIEVMYPPPYLDNEKSNGTIIHVKGKHLCPSPLFPGPSKPFWVLVWGGVLACYSLLVTVAFIIFWVRSKRSRLLHSDYMNMTPRRPGPTRKHYQPYAPPRDFAAYRS

SEQ ID NO: 19 - DNA, 321 bp, synthetic construct
  source 1..321: mol_type = other DNA
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 20 - protein, 234 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPWKSGDKPSLSARYV

SEQ ID NO: 21 - protein, 71 aa, synthetic construct
PTTTPAPRPPTPAPTIASQPLSLRPEACRPAAGGAVHTRGLDFACDIYIWAPLAGTCGVLLLSLVITLYCN

SEQ ID NO: 22 - DNA, 213 bp, synthetic construct
  source 1..213: mol_type = other DNA
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 23 - protein, 164 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 24 - protein, 164 aa, synthetic construct; identical to SEQ ID NO: 23

SEQ ID NO: 25 - protein, 112 aa, synthetic construct
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 337 bp, synthetic construct
  source 1..337: mol_type = other DNA
agagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcg

SEQ ID NO: 28 - DNA, 339 bp, synthetic construct
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 29 - protein, 251 aa, synthetic construct
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLGTKERDWCGPSPADLSPGASSVTPPAPAREPGHSPQIISFFLALTSTALLFLLFFLTLRFSWKRGRKKLLYIFKQPFMRPVQTTQEEDGCSCRFPEEEEGGCEL

SEQ ID NO: 30 - protein, 42 aa, synthetic construct
KRGRKKLLYIFKQPFMRPVQTTQEEDGCSCRFPEEEEGGCEL

SEQ ID NO: 31 - DNA, 126 bp, synthetic construct
  source 1..126: mol_type = other DNA
aaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 32 - protein, 276 aa, synthetic construct
MCVGARRLGRGPCAALLLLGLGLSTVTGLHCVGDTYPSNDRCCHECRPGNGMVSRCSRSQNTVCRPCGPGFYNDWSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 33 - protein, 196 aa, synthetic construct
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVWCILGCILICWLTKKKYSSSVHDPNGEYMFMRATAKKSRLTDVTL

SEQ ID NO: 34 - protein, 4 aa, synthetic construct
YVKM

SEQ ID NO: 35 - protein, 220 aa, synthetic construct
MACLGFQRHKAQLNLATRTWPCTLLFFLLFIPVFCKAMHVAQPAWLASSRGIASFVCEYASPGKATEVRVTVLRQADSQVTEVCAATYMMGNELTFLDDSICTGTSSGNQLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 36 - protein, 286 aa, synthetic construct
MQIPQAPWPVVWAVLQLGWRPGWFLDSPDRPWNPPTFSPALLWTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGWGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 37 - protein, 524 aa, synthetic construct
MWEAQFLGLLFLQPLWVAPVKPLQPGAEVPWWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 38 - protein, 368 aa, synthetic construct
MLGQWTLILLLLLKVYQGKGCQGSADHWSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 39 - protein, 289 aa, synthetic construct
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 40 - protein, 8 aa, synthetic construct
SIINFEKL

SEQ ID NO: 41 - protein, 6 aa, synthetic construct
HHHHHH

(19) United States
(12) Patent Application Publication
(10) Pub. No.: US 2025/0230215 A1
(43) Pub. Date: Jul. 17, 2025

(54) IMMUNE CELLS EXPRESSING GLUCOSE TRANSPORTER 5 (GLUT5) AND COMPOSITIONS AND METHODS INCLUDING THE SAME

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Kayvan R. Keshari, New York, NY (US); Justin Perry, New York, NY (US)

(21) Appl. No.: 18/708,163

(22) PCT Filed: Nov. 7, 2022
(86) PCT No.: PCT/US22/49114

§ 371 Date: May 7, 2024

Related U.S. Application Data
(60) Provisional application No. 63/276,911, filed on Nov. 8, 2021.
(60) Provisional application No. 63/338,626, filed on May 5, 2022.

(51) Int. Cl. C07K 14/705; A61K 40/11; A61K 40/42; A61P 35/00; C12N 5/0783

(52) U.S. Cl. CPC C07K 14/705; A61K 40/11; A61K 40/4202; A61P 35/00; C12N 5/0636; C12N 2510/00

(57) ABSTRACT

Provided herein are compositions, kits, and methods for manufacturing cells for adoptive cell therapy comprising engineered immune cells that overexpress Glucose Transporter 5 (GLUTS).

### TECHNICAL FIELD

This application is the U.S. National Stage Application of International Application No. PCT/US2022/049114, filed Nov. 7, 2022, which claims the benefit of and priority to U.S. Provisional Application Nos. 63/276,911, filed Nov. 8, 2021, and 63/338,626, filed May 5, 2022, the contents of which are incorporated herein by reference in their entireties.

### STATEMENT OF GOVERNMENT SUPPORT

This invention was made with government support under CA008748, awarded by National Institutes of Health. The government has certain rights in the invention.

### TECHNICAL FIELD

The present technology relates to are compositions, kits, and methods for manufacturing cells for adoptive cell therapy comprising engineered immune cells that overexpress glucose transporter 5 (GLUT5).

### BACKGROUND

The following description of the background of the present technology is provided simply as an aid in understanding the present technology and is not admitted to describe or constitute prior art to the present technology.

While checkpoint inhibitors and chimeric antigen receptor (CAR) T cells undergo widespread investigation as potential approaches to unleash the immune system's tumor-targeting abilities, the mechanisms by which these therapies fail is the subject of great debate. In the setting of solid tumors, it is believed that the microenvironment is hostile, excluding T cells and/or inhibiting their ability to proliferate or be activated. Significant recent work has demonstrated that one rationale for this explanation is the competition for nutrients in the microenvironment and the subsequent production of metabolic products that can inhibit T-cell function. To date, most research has focused on modulating suppression of T-cell function, better protecting T cells from the hostile microenvironment and selectively treating the tumor in order to modulate the production of toxic metabolites. These efforts have been met with significant challenges both in efficacy and translation, making novel approaches to enhancing the effectiveness of immunotherapeutic approaches a great unmet need.

Accordingly, there is an urgent need for methods and compositions that improve T cell cytotoxicity to tumor cells.

### SUMMARY OF THE PRESENT TECHNOLOGY

In one aspect, the present disclosure provides an engineered immune cell comprising a non-endogenous expression vector that includes a nucleic acid sequence encoding a Glucose Transporter 5 (GLUT5) amino acid sequence of SEQ ID NO: 1 or SEQ ID NO: 2. Additionally or alternatively, the engineered immune cell comprises a non-endogenous expression vector that includes a GLUT5 nucleic acid sequence of any one of SEQ ID NOs: 7-9. The engineered immune cell may be a T cell, a CD4+ T cell, a CD8+ T cell, a B cell, a natural killer (NK) cell, a natural killer T (NKT) cell, a dendritic cell, a myeloid cell, a monocyte, a macrophage, or a tumor-infiltrating immune cell. In some embodiments, the non-endogenous expression vector including the GLUT5 nucleic acid sequence is a plasmid, a cosmid, a bacmid, a bacterial artificial chromosome (BAC), a yeast artificial chromosome (YAC), a viral vector, or a retroviral vector. Additionally or alternatively, in some embodiments, the engineered immune cell lacks expression of a cytokine, such as TNFα, and/or T-Cell-Specific Transcription Factor (TCF-1).

Additionally or alternatively, in some embodiments of the engineered immune cell disclosed herein, the GLUT5 nucleic acid sequence is operably linked to an expression control sequence. The expression control sequence may be an inducible promoter, a constitutive promoter, a native GLUT5 promoter, or a heterologous promoter.

Additionally or alternatively, in some embodiments, the engineered immune cell further comprises a receptor that binds to a target antigen and/or a nucleic acid encoding the receptor. The receptor may be a native cell receptor, a non-native cell receptor, or a chimeric antigen receptor (CAR). In certain embodiments, the receptor is a T cell receptor.

Additionally or alternatively, in some embodiments, the CAR comprises (i) an extracellular antigen binding domain; (ii) a transmembrane domain; and (iii) an intracellular domain, wherein the extracellular antigen binding domain binds to the target antigen. In certain embodiments, the extracellular antigen binding fragment is a single-chain variable fragment (scFv). The transmembrane domain may comprise a CD8 transmembrane domain or a CD28 transmembrane domain. In certain embodiments, the intracellular domain comprises a CD3ζ signaling domain and optionally one or more costimulatory domains selected from a CD28 costimulatory domain, a 4-1BB costimulatory domain, an OX40 costimulatory domain, an ICOS costimulatory domain, a DAP-10 costimulatory domain, a PD-1 costimulatory domain, a CTLA-4 costimulatory domain, a LAG-3 costimulatory domain, a 2B4 costimulatory domain, a BTLA costimulatory domain, or any combination thereof.

Additionally or alternatively, in some embodiments of the engineered immune cell disclosed herein, the target antigen comprises a tumor antigen. Examples of tumor antigens include, but are not limited to, 5T4, alpha 5β1-integrin, 707-AP, A33, AFP, ART-4, B7H4, BAGE, Bcl-2, β-catenin, BCMA, Bcr-abl, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC16, MUM-1-B, myc, MUM-2, MUM-3, NA88-A, NYESO-1, NY-Eso-B, p53, proteinase-3, p190 minor bcr-abl, Pml/RARα, PRAME, progesterone receptor, PSA, PSCA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, or WT1.

In any of the preceding embodiments, the engineered immune cell is derived from an autologous donor or an allogenic donor.

In one aspect, the present disclosure provides a composition comprising an effective amount of any and all embodiments of the engineered immune cells disclosed herein and a pharmaceutically acceptable carrier.

In another aspect, the present disclosure provides a method of preparing immune cells for adoptive cell therapy comprising: isolating immune cells from a donor subject; and transducing the immune cells with a non-endogenous expression vector that includes a nucleic acid sequence encoding a Glucose Transporter 5 (GLUT5) amino acid sequence of SEQ ID NO: 1 or SEQ ID NO: 2, optionally wherein the nucleic acid sequence is any one of SEQ ID NOs: 7-9.

In yet another aspect, the present disclosure provides a method of treatment, comprising: isolating immune cells form a donor subject; transducing the immune cells with a non-endogenous expression vector that includes a nucleic acid sequence encoding a Glucose Transporter 5 (GLUT5) amino acid sequence of SEQ ID NO: 1 or SEQ ID NO: 2, optionally wherein the nucleic acid sequence is any one of SEQ ID NOs: 7-9; and administering the transduced immune cells to a recipient subject. In some embodiments, the donor subject and the recipient subject are the same. In other embodiments, the donor subject and the recipient subject are different.

Additionally or alternatively, in some embodiments of the methods disclosed herein, the immune cells isolated from the donor subject comprise one or more lymphocytes, such as T cells (e.g., CD8⁺ cytotoxic T cells, CD4⁺ T cells etc.), B cells, tumor infiltrating lymphocytes, natural killer cells, dendritic cells, myeloid cells, monocytes, macrophages and the like. In some embodiments, the T cell comprises a native T cell receptor (TCR), a non-native TCR, or a chimeric antigen receptor (CAR). In certain embodiments, the chimeric antigen receptor (CAR) binds to a tumor antigen.

In one aspect, the present disclosure provides a method for treating cancer or inhibiting tumor growth or metastasis in a subject in need thereof comprising administering to the subject an effective amount of any and all embodiments of the engineered immune cells disclosed herein or any and all embodiments of the compositions disclosed herein. In some embodiments, the cancer or tumor is selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof. The engineered immune cells may be administered pleurally, intravenously, subcutaneously, intranodally, intratumorally, intrathecally, intrapleurally or intraperitoneally.

Additionally or alternatively, in some embodiments, the method further comprises sequentially, separately, or simultaneously administering to the subject an additional cancer therapy such as chemotherapeutic agents, immune checkpoint inhibitors, monoclonal antibodies that specifically target tumor antigens, immune activating agents (e.g., interferons, interleukins, cytokines), oncolytic virus therapy and cancer vaccines.

Additionally or alternatively, in some embodiments, the method further comprises sequentially, separately, or simultaneously administering to the subject one or more of fructose; a pyruvate kinase M2 (PKM2) activator, DASA58, a ketohexokinase (KHK) inhibitor, or 6-(4-(2-Hydroxyethyl)piperazin-1-yl)-2-(3-(hydroxymethyl)-piperidin-1-yl)-4-(trifluoromethyl)nicotinonitrile.

Also disclosed herein are kits comprising an expression vector that includes a nucleic acid sequence encoding a Glucose Transporter 5 (GLUT5) amino acid sequence of SEQ ID NO: 1 or SEQ ID NO: 2, and instructions for transducing immune cells with the expression vector. Additionally or alternatively, in some embodiments, the nucleic acid sequence is any one of SEQ ID NOs: 7-9. In certain embodiments, the kits further comprise a vector encoding an engineered T-cell receptor (TCR) or other cell-surface ligand that binds to a target antigen. Additionally or alternatively, in some embodiments, the kits further comprise one or more of: fructose, a pyruvate kinase M2 (PKM2) activator, a ketohexokinase (KHK) inhibitor, or an additional anti-cancer therapeutic agent.

### BRIEF DESCRIPTION OF THE DRAWINGS

FIGS. 1A-1B provide schematics of fructose metabolism. As shown in FIG. 1A, Fructose is metabolized to fructose-1-phosphoate by the enzyme ketohexokinase (KHK), or to fructose-6-phosphate by the enzyme hexokinase (HK). The HK and related pathways are further illustrated in FIG. 1B.

FIGS. 2A-2C show that metabolite(s) can be traced in vivo. FIG. 2A illustrated metabolism of hyperpolarized [1-¹³C]pyruvate in vivo. FIG. 2B provides representative T2 weighted axial images of a patient with Gleason Score 3 and 4/5 prostate cancer with the color overlays of HP ¹³C lactate generated from infusions of HP pyruvate. The maximum lactate ratio maps show similar spatial distributions to the regions marked on histology slides by a pathologist. Regions of high-grade tumor (Gleason 4/5) show higher ratios than those of low-grade tumor (Gleason 3). Specifically, the left panel provides a representative image of Gleason Grade 3 tumor showing normalized lactate ratios less than 0.5, while the right panel provides a representative image of Gleason Grade 4/5 tumor showing normalized lactate ratios higher than 0.5. FIG. 2C shows that the lactate ratio increases with tumor Gleason Score. The ratios are significantly lower in normal tissue than in tumors (p=0.0001 for Grade 3, p<0.0001 for Gleason Score ≥4). FIGS. 2B-2C adapted from Granlund et al. Cell Metab. 2020 Jan. 7; 31(1):105-114.e3.

FIGS. 3A-3G show that leukemic cells proliferate with fructose in the absence of glucose, and the metabolic fate of fructose is determined by GLUT5. FIG. 3A provides growth curves of MOLM13 and K562 cells in the culture media with 10 mM of glucose or fructose (mean±S.D., n=4 biologically independent samples). FIG. 3B provides a comparison of growth rates of four leukemic cell lines. The growth rate in the fructose-rich condition (the right box of the data plotted for each cell line) was normalized to that in the glucose-rich condition (the left box of the data plotted for each cell line). The box-whisker plot shows the center line as median, the box limits as upper and lower quartiles, and the whiskers to minimum and maximum values). FIG. 3C provides an immunoblot analysis of metabolic enzymes and GLUT5. FIG. 3D shows changes in metabolites in the media after 2-day incubation (mean±S.D., n=3 biologically independent samples). For each sets of bars, the left one plots the data in the glucose-rich condition, while the right one plots the data in the fructose-rich condition. FIG. 3E provides ¹³C-NMR spectra of the media after four leukemic cells were incubated with 10 mM of [2-¹³C]glucose or fructose for 2 days. FIG. 3F provides a quantitative comparison of the peak area of [2-¹³C]glycine normalized to that of [2-¹³C] lactate in the same spectrum in FIG. 3E (mean±S.D., n=3 biologically independent samples). For each sets of bars, the upper one plots the data in the glucose-rich condition, while the lower one plots the data in the fructose-rich condition. FIG. 3G provides a comparison of intracellular metabolites level in MOLM13 cells incubated with 10 mM of [U-¹³C]glucose (left for each panel) or fructose (right for each panel). The total pool size and fully-enriched pool size (m+3 for 3-PG, serine, and lactate; m+2 for glycine) were compared (mean±S.D., n=3 biologically independent samples). All statistical analyses were conducted with unpaired two-tailed t-test. Figure adapted from Jeong et al. Cell Metab. 2021 Jan. 5; 33(1):145-159.e6.

FIGS. 4A-4E show that PKM2 inactivation in the fructose-rich condition leads to the accumulation of the glycolytic intermediate, providing the precursor for SSP. FIG. 4A provides the relative level of intracellular metabolites, fructose-1,6-bisphosphoate (F-1,6-P), glyceraldehyde-3-phosphate (G-3-P), dihydroxyacetone phosphate (DHAP), 3-phosphoglycerate (3-PG), and phosphoenolpyruvate (PEP), in MOLM13 cells cultured in the media with 10 mM of [U-¹³C]glucose (left for each metabolite) or fructose (right for each metabolite) for 2 days (mean±S.D., n=3 biologically independent samples). FIG. 4B provides schematics displaying the mechanism of the metabolic changes between the glucose-rich and fructose-rich conditions. FIG. 4C provides ¹³C-NMR spectra of the media after 2-day culture of MOLM13 cells in [2-¹³C] fructose with different doses of PKM2 activator, DASA58. FIG. 4D provides relative changes in metabolites in the media from FIG. 4C. The metabolite levels were normalized to the level with control treatment (DMSO, 0.1%) (mean±S.D., n=3 biologically independent samples). FIG. 4E provides a comparison of growth rates of MOLM13 cells in the media with 10 mM of glucose or fructose with PKM2 activator, DASA58. (mean±S.D., n=3 biologically independent samples). All statistical analyses were conducted with unpaired two-tailed t-test. Figure adapted from Jeong et al. Cell Metab. 2021 Jan. 5; 33(1):145-159.e6.

FIGS. 5A-5E show overexpression of GLUT5 and its effect on fructose metabolism. FIG. 5A shows that GLUT5 was overexpressed in K562 cells (GLUT5-OV), which was confirmed using flow cytometry. CTRL-OV, K562 cells with empty vector overexpression; GLUT5-OV, K562 cells with GLUT5 overexpression. FIG. 5B shows that changes in metabolites in the media after K562 GLUT5-OV (right bar of each set) and CTRL-OV (left bar of each set) cells were incubated with 10 mM fructose (no glucose) for 2 days (mean±S.D., n=3 biologically independent samples). FIG. 5C shows that ¹³C-NMR spectra of the media after K562 GLUT5-OV and CTRL-OV cells were incubated with 10 mM of [2-¹³C] fructose (no glucose) for 2 days. FIG. 5D provides a quantitative comparison of the peak area of [2-¹³C]glycine normalized to that of [2-¹³C] lactate in the same spectrum in FIG. 5C (mean±S.D., n=3 biologically independent samples). Data from K562 GLUT5-OV is plotted in the right bar; while data from CTRL-OV is plotted in the left bar. All statistical analyses were conducted with unpaired two-tailed t-test. FIG. 5E shows that GLUT5 was overexpressed in primary human T-cells using the same construct and analyzed via flow cytometry. Figure adapted from Jeong et al. Cell Metab. 2021 Jan. 5; 33(1):145-159.e6.

FIGS. 6A-6E show modeling T-cell exhaustion in vitro. FIG. 6A provides a schematic for inducing T-cell exhaustion via chronic stimulation in vitro. Figure discloses SEQ ID NO: 40. FIG. 6B provides that chronically stimulated T-cells fail to produce cytokines (e.g., TNFα) and upregulate immune checkpoints (e.g., PD-1). FIG. 6C provides that chronically stimulated T-cells lose expression of the T-Cell-Specific Transcription Factor (TCF-1) and upregulate expression of the exhaustion-associated transcription factor TOX. FIG. 6D provides that chronically stimulated cells are highly enriched in genes upregulated in exhausted tumor infiltrating CD8 T-cells from mouse models and patients as well as T-cells from mice bearing chronic viral infections, but not significantly enriched in genes upregulated in anergic T-cells. FIG. 6E provides that chronically stimulated T-cells lose the ability to suppress tumor growth in vivo.

FIGS. 7A-7D show that chronically stimulated T-cells exhibit impaired glucose metabolism. FIG. 7A shows a schematic for isotope tracing of uniformly ¹³C-labeled glucose during acute and chronic stimulation of T-cells. FIG. 7B shows fractional labeling of tricarboxylic acid (TCA) cycle metabolites in acutely (left bar of each set) and chronically (right bar of each set) stimulated T-cells using uniformly [¹³C]glucose. Ions used for quantification of metabolite levels are as follows: d5-2HG m/z 354; αKG, m/z 304; aspartate, m/z 334; citrate, m/z 465; fumarate, m/z 245; glutamate, m/z 363; malate, m/z 335 and succinate, m/z 247. All peaks are manually inspected and verified relative to known spectra. FIGS. 7C-7D shows chronically stimulated T-cells require high amounts of available glucose to sustain growth. Data from the acutely stimulated T-cells is plotted in FIG. 7C; while data from the chronically stimulated T-cells is plotted in FIG. 7D.

FIGS. 8A-8B show regulation of T-cell killing by nutrient availability. FIG. 8A provides a schematic for T-cell killing assay. OT-I TCR transgenic CD8+ T-cells are co-cultured with B16 cells expressing a luciferase reporter that have been pulsed with SIINFEKL (SEQ ID NO: 40) peptide in media containing varying levels of extracellular glucose. 24 hours later, luminescence is measured. FIG. 8B shows decreased target cell killing by T-cells when cultured in 1 mM glucose.

FIGS. 9A-9H show that hyperpolarized [2-¹³C] fructose has the potential to trace in vivo metabolism even with a short lifetime of 13 s, and visualized its conversion to F6P. FIG. 9A provides the mechanism for transport by GLUT5 and the first step of metabolism of fructose to fructofuranose-6-phosphate by hexokinase. FIG. 9B provides a T₂-weighted image of a transgenic model of prostate cancer (TRAMP) mouse with tumor only on the right side of the prostate. Metabolic images of total hyperpolarized [2-¹³C] fructose resonances (FIG. 9C) and the composite 0-fructofuranose-6-phosphate and β-fructofuranose (FIG. 9D) are shown overlaid on the T₂ weighted image. Resonances corresponding to the β-fructopyranose and composite p-fructofuranose-6-phosphate and p-fructofuranose are shown in the corresponding spectral array (FIG. 9E). FIGS. 9A-9E adapted from Keshari et al. J Am Chem Soc. 2009 Dec. 9; 131(48):17591-6. The yellow area covering the rectangles numbered with 1 to 4 demonstrates a region of tumor, compared to a region of benign prostate tissue in red covering the rectangles numbered with 5 to 8. An unassigned, spurious, low signal-to-noise resonance appears at 115 ppm. FIG. 9F provides a synthetic scheme for U-²H-[2-¹³C] fructose. The C2 is annotated by an C. FIG. 9G provides representative dynamic spectra of HP [2-¹³C] fructose dissolved in D₂O and HP U-²H-[2-¹³C] fructose dissolved in D20. Both solutions were approximately 30 mM and spectra acquired using 5° flip angle every 3 s. FIG. 9H provides an integration of peak intensity in time shows a dramatic extension of lifetime for HP U-²H-[2-¹³C] fructose in D20 as compared to HP [2-¹³C] fructose.

FIGS. 10A-10D show sensitive and nondestructive analysis using the hyperpolarized micro-NMR platform. FIG. 10A provides titration data of the flux metric ξ of UOK262 cells. FIG. 10B provides profiling of the flux metric in five different cell lines: UOK262 (kidney cancer), U87 (glioblastoma), Jurkat (acute T cell leukemia), K562 (CML), and HK-2 (kidney). FIG. 10C provides NMR spectra of hyperpolarized metabolites acquired from malignant cancer cells (UOK262) and nonmalignant ones (HK-2). For each assay, 10⁵ cells were used. FIG. 10D provides a comparison of viability before and after assays. Two cancer cell lines (K562 and UOK262) were tested. The “Before” measurement was conducted 15 min before the HMRS assay, and the “After” measurement was conducted 15 min after the HMRS assay. All measurements were performed in duplicate. Figure adapted from Jeong et al. Sci Adv. 2017 Jun. 16; 3(6):e1700341.

FIGS. 11A-11C provide fructose converted to fructose-1-phosphate (F1P) and later to lactate in vivo measured in transformed hepatocytes (HepG2 cells) using [2-¹³C] fructose. FIG. 11A provides ¹³C NMR spectra of media containing [2-¹³C] fructose exposed to HepG2 cells. FIG. 11B provides a quantification of fructose consumption by HepG2. FIG. 11C provides ¹³C NMR spectra of a representative HepG2 cell extract demonstrating high levels of [2-¹³C]F1P derived from the labeled fructose in media and easily resolved. The subsequent decrease in F1P with treatment using the KHKi is also readily demonstrated with treatment at 4 hr in vitro, highlighting that this drug is on target.

FIG. 12 shows liver and B16 melanoma fructose concentrations at 1 hr post-IP injection of fructose tumor bearing mice. Tumor fructose is well within the range necessary to feed T-cells over expressing GLUT5, demonstrating that a bolus of fructose can dramatically increase the tumor fructose. Further this supports that HP fructose reaches the tumor and provides a platform for imaging.

FIG. 13 shows that primary human T-cells are unable to metabolize fructose at the levels of glucose to drive glycolysis.

FIG. 14 demonstrates overexpression of SLC2A5 (GLUT5) in T-cells of OT-1 mice.

FIG. 15 shows detected levels of [2-¹³C] lactate after incubating T-cells of OT-1 mice with [2-¹³C]glucose (labeled as glucose in the figure) or [2-¹³C] fructose (labelled as fructose in the figure) for 8 hours. The tested T-cells overexpress GLUT5 (labeled as Glut5) or not (labeled as mock).

FIGS. 16A-16B show that primary macrophages bearing GLUT5 survive and differentiate on fructose. As shown in FIG. 16A, primary macrophages are unable to use fructose as an energy source and begin to undergo de-differentiation and cell death after 24 h of treatment. On the other hand, FIG. 16B shows that primary macrophages stably (over)expressing a novel GLUT5 construct survive, differentiate, and proliferate on fructose even in the absence of glucose.

FIGS. 17A-17B show that expression of GLUT5 in macrophages allows for fructose use during phagocytosis of triple-negative breast cancer cells (TNBCs). FIG. 17A provides representative flow cytometry results; while FIG. 17B provides a bar graph quantifying the phagocytosis.

FIG. 18A shows a schematic of the experimental design for FIG. 18B. FIG. 18B shows the quantification of B16 luminescence post incubation with either control or GLUT5 expressing T cells. FIG. 18C shows the analysis of glycolytic intermediates in Empty Vector (EV) or GLUT5 (GT5) T cells after incubation in isotopic fructose or glucose. FIG. 18D shows a representative image of flow cytometry analysis of CFSE dye incorporation into EV or GLUT5 T cells in 10 mM fructose. Quantification of the same experiment performed in either 10 mM glucose, 10 mM fructose or 2 mM glucose+2 mM fructose (LG+LF). FIG. 18E shows a depiction of the proposed mouse experiment.

FIG. 19A shows a schematic of the immunosuppressive tumor microenvironment (TME). FIG. 19B shows a schematic of how glucose is limiting in the (TME) which makes T cells exhausted; fructose is present in the TME and is not generally consumed by cancer cells or immune cells. FIG. 19C shows that expression of fructose transporter GLUT5 (GT5) on T cells allowed them to consume fructose and metabolize it, producing lactate.

FIG. 20A shows that wild-type (WT) T cells do not metabolize fructose as evident through decreased production of glycolytic intermediates, GA3P, PEP and lactate when grown in fructose compared to glucose. FIG. 20B shows that GT5-expressing T cells grown in fructose are able to produce GA3P, PEP and lactate, which is comparable to WT T cells grown in high glucose media. FIG. 20C: GT5 or WT T cells were grown with B16 melanoma cells in glucose or fructose, and their ability to kill was evaluated. FIG. 20D: T cells were either isolated from B6 mice or Balb/c mice, engineered to express GT5, and grown with 4T1 breast cancer cells which come from Balb/c background. The ability of B6 GT5 T cells to kill based on MHC mismatch was evaluated in glucose and fructose media.

FIGS. 21A-21B: The ability of GT5 cells to kill in vivo was evaluated: B16 melanoma cells were injected into mouse flanks and either WT (EV) or GT5 T cells were injected one week post tumor implantation. Figures on the bottom show the progression of individual mice and their representative MRI images.

FIG. 22A: GT5 was introduced into macrophages and subsequently cultured in fructose or glucose prior to co-culture with apoptotic human breast cancer cells. Control cells (cells not given GT5) were generally unable to use fructose to perform phagocytosis of apoptotic breast cancer cells (termed efferocytosis; compare orange bars to white bars). On the other hand, macrophages with GT5 not only were able to efficiently perform efferocytosis of apoptotic breast cancer cells, they did so better than any condition tested (compare red bar to white and orange bars). FIG. 22B: Similar to FIG. 22A, except instead live human breast cancer cells were concurrently labeled with the antibody CD47, allowing macrophages to perform antibody-mediated phagocytosis. Importantly, macrophages with GT5 exhibited significantly enhanced antibody-mediated phagocytosis of CD47-labeled human breast cancer cells than all other conditions tested (compared red bar to white and orange bars). FIG. 22C: The ability of GT5 macrophages to prevent progression of two orthotopic mouse models of human breast cancer. All mice were treated with CD47 antibody beginning day 7 after implantation. Additionally, tumor-size matched mice were administered either macrophages with a control construct or GT5. Both immune checkpoint blockade (ICB)-sensitive (E0771) and -insensitive (4T1) orthotopic models significantly responded to GT5 macrophage infusion without the need for ICB therapy.

### DETAILED DESCRIPTION

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

Current strategies to selectively supply engineered immune cells (e.g., T cells) in vivo with a substrate for energy are limited and a better mechanistic understanding of immune cell (e.g., T-cell) metabolism is needed. Characterizing immunometabolism pathways provides a unique understanding of tumor biochemistry and has a significant impact in the setting of immunotherapies. Without wishing to be bound by the theory, while many tumor cells can take up glucose and compete with immune cells (e.g., T cells), very few can utilize fructose efficiently. Thus, the present disclosure provides strategies to modulate glycolysis in immune cells (e.g., T cells) using fructose as a substrate and enhancing their cytotoxicity to tumor cells.

### Definitions

As it would be understood, the section or subsection headings as used herein is for organizational purposes only and are not to be construed as limiting and/or separating the subject matter described.

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1.0 or 0.1, as appropriate or alternatively by a variation of +/−20% or +/−15%, or alternatively 10% or alternatively 5% or alternatively 2%. As will be understood by one skilled in the art, for any and all purposes, all ranges disclosed herein also encompass any and all possible subranges and combinations of subranges thereof. Furthermore, as will be understood by one skilled in the art, a range includes each individual member.

As used herein, the singular forms “a”, “an” and “the” are intended to include the plural forms as well, unless the context clearly indicates otherwise. For example, the term “a cell” includes a plurality of cells, including mixtures thereof.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value.

As used herein, the term “administration” of an agent to a subject includes any route of introducing or delivering the agent to a subject to perform its intended function. Administration can be carried out by any suitable route, including, but not limited to, intravenously, intramuscularly, intraperitoneally, subcutaneously, and other suitable routes as described herein. Administration includes self-administration and the administration by another. “Administration” of a cell or vector or other agent and compositions containing same can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. In some embodiments, administering or a grammatical variation thereof also refers to more than one doses with certain interval. In some embodiments, the interval is 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application. In some embodiments, the administration is an infusion (for example to peripheral blood of a subject) over a certain period of time, such as about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours or longer.

As used herein “adoptive cell therapeutic composition” refers to any composition comprising cells suitable for adoptive cell transfer. In exemplary embodiments, the adoptive cell therapeutic composition comprises a cell type selected from a group consisting of a tumor infiltrating lymphocyte (TIL), TCR (i.e. heterologous T-cell receptor) modified lymphocytes (e.g., eTCR T cells and caTCR T cells) and CAR (i.e. chimeric antigen receptor) modified lymphocytes (e.g., CAR T cells). In another embodiment, the adoptive cell therapeutic composition comprises a cell type selected from a group consisting of T-cells, CD8+ cells, CD4+ cells, NK-cells, delta-gamma T-cells, regulatory T-cells and peripheral blood mononuclear cells. In another embodiment, TILs, T-cells, CD8+ cells, CD4+ cells, NK-cells, delta-gamma T-cells, regulatory T-cells or peripheral blood mononuclear cells form the adoptive cell therapeutic composition. In one embodiment, the adoptive cell therapeutic composition comprises T cells.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form, and a second plurality of amino acids are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

As used herein, the term “analog” refers to a structurally related polypeptide or nucleic acid molecule having the function of a reference polypeptide or nucleic acid molecule.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983)). Antibodies may comprise whole native antibodies, monoclonal antibodies, human antibodies, humanized antibodies, camelised antibodies, multispecific antibodies, bispecific antibodies, chimeric antibodies, Fab, Fab′, single chain V region fragments (scFv), single domain antibodies (e.g., nanobodies and single domain camelid antibodies), VNAR fragments, Bi-specific T-cell engager (BiTE) antibodies, minibodies, disulfide-linked Fvs (sdFv), and anti-idiotypic (anti-Id) antibodies, intrabodies, fusion polypeptides, unconventional antibodies and antigen binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA, and IgY), class (e.g., IgG1, IgG2, IgG3, IgG4, IgA1, and IgA2), or subclass.

In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, C_{H}1, C_{H}2, and C_{H}3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. As used herein interchangeably, the terms “antigen binding portion”, “antigen binding fragment”, or “antigen binding region” of an antibody, refer to the region or portion of an antibody that binds to the antigen and which confers antigen specificity to the antibody; fragments of antigen binding proteins, for example antibodies, include one or more fragments of an antibody that retain the ability to specifically bind to an antigen. It has been shown that the antigen binding function of an antibody can be performed by fragments of a full-length antibody. Examples of antigen binding portions encompassed within the term “antibody fragments” of an antibody include a Fab fragment, a monovalent fragment consisting of the V_{L}, V_{H}, C_{L} and C_{H}1 domains; a F(ab)₂ fragment, a bivalent fragment comprising two Fab fragments linked by a disulfide bridge at the hinge region; a Fd fragment consisting of the V_{H} and C_{H}1 domains; a Fv fragment consisting of the V_{L} and V_{H} domains of a single arm of an antibody; a dAb fragment (Ward et al., Nature 341: 544-546 (1989)), which consists of a V_{H} domain; and an isolated complementarity determining region (CDR). An “isolated antibody” or “isolated antigen binding protein” is one which has been identified and separated and/or recovered from a component of its natural environment. “Synthetic antibodies” or “recombinant antibodies” are generally generated using recombinant technology or using peptide synthetic techniques known to those of skill in the art.

Antibodies and antibody fragments can be wholly or partially derived from mammals (e.g., humans, non-human primates, goats, guinea pigs, hamsters, horses, mice, rats, rabbits and sheep) or non-mammalian antibody producing animals (e.g., chickens, ducks, geese, snakes, and urodele amphibians). The antibodies and antibody fragments can be produced in animals or produced outside of animals, such as from yeast or phage (e.g., as a single antibody or antibody fragment or as part of an antibody library).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science 242:423-426 (1988); and Huston et al., Proc. Natl. Acad. Sci. 85: 5879-5883 (1988). These antibody fragments are obtained using conventional techniques known to those of ordinary skill in the art, and the fragments are screened for utility in the same manner as are intact antibodies.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., about 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen binding domain. In certain embodiments, the linker comprises amino acids having GGGGSGGGGSGGGGS (SEQ ID NO: 3). In certain embodiments, the nucleic acid sequence encoding the amino acid sequence of SEQ ID NO: 3 is ggcggcggcggatctggaggtggtggctcaggtggcggaggctcc (SEQ ID NO: 4).

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 85:5879-5883 (1988)). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 27(6):455-51 (2008); Peter et al., J Cachexia Sarcopenia Muscle (2012); Shieh et al., J Imunol 183(4):2277-85 (2009); Giomarelli et al., Thromb Haemost 97(6):955-63 (2007); Fife et al., J Clin Invst 116(8):2252-61 (2006); Brocks et al., Immunotechnology 3(3): 173-84 (1997); Moosmayer et al., Ther Immunol 2(10):31-40 (1995). Agonistic scFvs having stimulatory activity have been described (see, e.g., Peter et al., J Biol Chem 25278(38):36740-7 (2003); Xie et al., Nat Biotech 15(8):768-71 (1997); Ledbetter et al., Crit Rev Immunol 17(5-6):427-55 (1997); Ho et al., Bio Chim Biophys Acta 1638(3):257-66 (2003)).

As used herein, an “antigen” refers to a molecule to which an antibody can selectively bind. The target antigen may be a protein (e.g., an antigenic peptide), carbohydrate, nucleic acid, lipid, hapten, or other naturally occurring or synthetic compound. An antigen may also be administered to an animal subject to generate an immune response in the subject.

As used herein, a “cancer” is a disease state characterized by the presence in a subject of cells demonstrating abnormal uncontrolled replication and in some aspects, the term may be used interchangeably with the term “tumor.” The term “cancer or tumor antigen” refers to an antigen known to be associated and expressed in a cancer cell or tumor cell (such as on the cell surface) or tissue, and the term “cancer or tumor targeting antibody” refers to an antibody that targets such an antigen. In some embodiments, the cancer or tumor antigen is not expressed in a non-cancer cell or tissue. In some embodiments, the cancer or tumor antigen is expressed in a non-cancer cell or tissue at a level significantly lower compared to a cancer cell or tissue.

In some embodiments, the cancer is selected from: circulatory system, for example, heart (sarcoma [angiosarcoma, fibrosarcoma, rhabdomyosarcoma, liposarcoma], myxoma, rhabdomyoma, fibroma, and lipoma), mediastinum and pleura, and other intrathoracic organs, vascular tumors and tumor-associated vascular tissue; respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); gastrointestinal stromal tumors and neuroendocrine tumors arising at any site; genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), placenta, vagina (clear cell carcinoma, squamous cell carcinoma, botryoid sarcoma (embryonal rhabdomyosarcoma), fallopian tubes (carcinoma) and other sites associated with female genital organs; penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Kaposi's sarcoma, moles dysplastic nevi, lipoma, angioma, dermatofibroma, and keloids; adrenal glands: neuroblastoma; and other tissues comprising connective and soft tissue, retroperitoneum and peritoneum, eye, intraocular melanoma, and adnexa, breast, head or neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites. In some embodiments, the cancer is a colon cancer, colorectal cancer or rectal cancer. In some embodiments, the cancer is a lung cancer. In some embodiments, the cancer is a pancreatic cancer. In some embodiments, the cancer is an adenocarcinoma, an adenocarcinoma, an adenoma, a leukemia, a lymphoma, a carcinoma, a melanoma, an angiosarcoma, or a seminoma.

In some embodiments, the cancer is a solid tumor. In other embodiments, the cancer is not a solid tumor. In some embodiments, the cancer is from a carcinoma, a sarcoma, a myeloma, a leukemia, or a lymphoma. In some embodiments, the cancer is a primary cancer or a metastatic cancer. In some embodiments, the cancer is a relapsed cancer. In some embodiments, the cancer reaches a remission, but can relapse. In some embodiments, the cancer is unresectable.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 10,000 cells, at least about 100,000 cells, at least about 1×10⁶ cells, at least about 1×10⁷ cells, at least about 1×10⁸ cells, at least about 1×10⁹ cells, at least about 1×10¹⁰ cells, at least about 1×10¹¹ cells, at least about 1×10¹² cells, or more cells expressing similar or different phenotypes.

As used herein, the term “chimeric co-stimulatory receptor” or “CCR” refers to a chimeric receptor that binds to an antigen and provides co-stimulatory signals, but does not provide a T-cell activation signal.

As used herein, a “cleavable peptide”, which is also referred to as a “cleavable linker,” means a peptide that can be cleaved, for example, by an enzyme. One translated polypeptide comprising such cleavable peptide can produce two final products, therefore, allowing expressing more than one polypeptides from one open reading frame. One example of cleavable peptides is a self-cleaving peptide, such as a 2A self-cleaving peptide. 2A self-cleaving peptides, is a class of 18-22 aa-long peptides, which can induce the cleaving of the recombinant protein in a cell. In some embodiments, the 2A self-cleaving peptide is selected from P2A, T2A, E2A, F2A and BmCPV2A. See, for example, Wang Y, et al. Sci Rep. 2015; 5:16273. Published 2015 Nov. 5. As used herein, the terms “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X-N-P-G-P (SEQ ID NO: 5), wherein X refers to any amino acid generally thought to be self-cleaving.

As used herein, “complementary” sequences refer to two nucleotide sequences which, when aligned anti-parallel to each other, contain multiple individual nucleotide bases which pair with each other. Paring of nucleotide bases forms hydrogen bonds and thus stabilizes the double strand structure formed by the complementary sequences. It is not necessary for every nucleotide base in two sequences to pair with each other for sequences to be considered “complementary”. Sequences may be considered complementary, for example, if at least 30%, 40%, 50%, 55%, 60%, 65%, 70%, 75%, 80%, 85%, 90%, 95%, 98%, 99%, or 100% of the nucleotide bases in two sequences pair with each other. In some embodiments, the term complementary refers to 100% of the nucleotide bases in two sequences pair with each other. In addition, sequences may still be considered “complementary” when the total lengths of the two sequences are significantly different from each other. For example, a primer of 15 nucleotides may be considered “complementary” to a longer polynucleotide containing hundreds of nucleotides if multiple individual nucleotide bases of the primer pair with nucleotide bases in the longer polynucleotide when the primer is aligned anti-parallel to a particular region of the longer polynucleotide. Nucleotide bases paring is known in the field, such as in DNA, the purine adenine (A) pairs with the pyrimidine thymine (T) and the pyrimidine cytosine (C) always pairs with the purine guanine (G); while in RNA, adenine (A) pairs with uracil (U) and guanine (G) pairs with cytosine (C). Further, the nucleotide bases aligned anti-parallel to each other in two complementary sequences, but not a pair, are referred to herein as a mismatch.

A “composition” is intended to mean a combination of active agent and another compound or composition, inert (for example, a nanoparticle, detectable agent or label) or active, such as an adjuvant, diluent, binder, stabilizer, buffers, salts, lipophilic solvents, preservative, adjuvant or the like and include carriers, such as pharmaceutically acceptable carriers. In some embodiments, the carrier (such as the pharmaceutically acceptable carrier) comprises, or consists essentially of, or yet further consists of a nanoparticle, such as an polymeric nanoparticle carrier or an lipid nanoparticle that can be used alone or in combination with another carrier, such as an adjuvant or solvent. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol. A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, the term, “co-stimulatory signaling domain,” or “co-stimulatory domain”, refers to the portion of the CAR comprising the intracellular domain of a co-stimulatory molecule. Co-stimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD28 and 4-1BB, other costimulatory domains are contemplated for use with the CARs described herein. The inclusion of one or more co-stimulatory signaling domains can enhance the efficacy and expansion of T cells expressing CAR receptors. The intracellular signaling and co-stimulatory signaling domains can be linked in any order in tandem to the carboxyl terminus of the transmembrane domain.

As used herein, the phrase “derived” means isolated, purified, mutated, or engineered, or any combination thereof. For example, an immune cell derived from a donor refers to the immune cell isolated from a biological sample of the donor and optionally engineered.

As used herein, the term “effective amount” or “therapeutically effective amount” refers to a quantity of an agent sufficient to achieve a beneficial or desired clinical result upon treatment. In the context of therapeutic applications, the amount of a therapeutic agent administered to the subject can depend on the type and severity of the disease or condition and on the characteristics of the individual, such as general health, age, sex, body weight, effective concentration of the engineered immune cells administered, and tolerance to drugs. It can also depend on the degree, severity, and type of disease. The skilled artisan will be able to determine appropriate dosages depending on these and other factors. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art.

As used herein, the term “excipient” refers to a natural or synthetic substance formulated alongside the active ingredient of a medication, included for the purpose of long-term stabilization, bulking up solid formulations, or to confer a therapeutic enhancement on the active ingredient in the final dosage form, such as facilitating drug absorption, reducing viscosity, or enhancing solubility.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g., by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell.

As used herein, an “expression vector” includes vectors capable of expressing DNA that is operably linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, the term “heterologous nucleic acid molecule or polypeptide” refers to a nucleic acid molecule (e.g., a cDNA, DNA or RNA molecule) or polypeptide that is either not normally expressed or is expressed at an aberrant level in a cell or sample obtained from a cell. This nucleic acid can be from another organism, or it can be, for example, an mRNA molecule that is not normally expressed in a cell or sample.

As used herein, a “host cell” is a cell that is used to receive, maintain, reproduce and amplify an expression vector. A host cell also can be used to express the polypeptide encoded by the expression vector. The nucleic acid contained in the expression vector is replicated when the host cell divides, thereby amplifying the nucleic acids. In some embodiments, the host cell as disclosed herein is a eukaryotic cell or a prokaryotic cell. In some embodiments, the host cell is a human cell. In some embodiments, the host cell is a cell line, such as a human embryonic kidney 293 cell (HEK 293 cell or 293 cell), or a 293T cell. These cells are commercially available, for example, from the American Type Culture Collection (ATCC).

As used herein, the term “increase” or “enhance” means to alter positively by at least about 5%, including, but not limited to, alter positively by about 5%, by about 10%, by about 25%, by about 30%, by about 50%, by about 75%, or by about 100%.

As used herein, the term “immune cell” refers to any cell that plays a role in the immune response of a subject. Immune cells are of hematopoietic origin, and include lymphocytes, such as B cells and T cells; natural killer cells; myeloid cells, such as monocytes, macrophages, dendritic cells, eosinophils, neutrophils, mast cells, basophils, and granulocytes. As used herein, the term “engineered immune cell” refers to an immune cell that is genetically modified. As used herein, the term “native immune cell” refers to an immune cell that naturally occurs in the immune system.

The terms “ketohexokinase,” and “KHK” are used interchangeably and refer to a ketohexokinase that catalyzes conversion of fructose to fructose-1-phosphate. The product of this gene is the first enzyme with a specialized pathway that catabolizes dietary fructose. In some embodiments, the KHK is a human KHK. Non-limiting exemplary sequences of this protein or the underlying gene can be found under Gene Cards ID: GC02P027086, HGNC: 6315, NCBI Entrez Gene: 3795, Ensembl: ENSG00000138030, OMIM®: 614058, or UniProtKB/Swiss-Prot: P50053, each of which is incorporated by reference herein in its entirety.

As used herein, the term “ligand” refers to a molecule that binds to a receptor. In particular, the ligand binds a receptor on another cell, allowing for cell-to-cell recognition and/or interaction.

As used herein, the term “linker” refers to any amino acid sequence comprising from a total of 1 to 200 amino acid residues; or about 1 to 10 amino acid residues, or alternatively 8 amino acids, or alternatively 6 amino acids, or alternatively 5 amino acids that may be repeated from 1 to 10, or alternatively to about 8, or alternatively to about 6, or alternatively to about 5, or alternatively, to about 4, or alternatively to about 3, or alternatively to about 2 times. For example, the linker may comprise up to 15 amino acid residues consisting of a pentapeptide repeated three times. In one embodiment, the linker sequence is a (G4S)n (SEQ ID NO: 6), wherein n is 1, or 2, or 3, or 4, or 5, or 6, or 7, or 8, or 9, or 10, or 11, or 12, or 13, or 14, or 15.

The term “lymphocyte” refers to all immature, mature, undifferentiated, and differentiated white blood cell populations that are derived from lymphoid progenitors including tissue specific and specialized varieties, and encompasses, by way of non-limiting example, B cells, T cells, NKT cells, and NK cells. In some embodiments, lymphocytes include all B cell lineages including pre-B cells, progenitor B cells, early pro-B cells, late pro-B cells, large pre-B cells, small pre-B cells, immature B cells, mature B cells, plasma B cells, memory B cells, B-1 cells, B-2 cells, and anergic AN1/T3 cell populations.

As used herein, “operably linked” with reference to nucleic acid sequences, regions, elements or domains means that the nucleic acid regions are functionally related to each other. For example, a nucleic acid encoding a leader peptide can be operably linked to a nucleic acid encoding a polypeptide, whereby the nucleic acids can be transcribed and translated to express a functional fusion protein, wherein the leader peptide affects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

“Optional” or “optionally” means that the subsequently described circumstance may or may not occur, so that the description includes instances where the circumstance occurs and instances where it does not.

As used herein, the “percent homology” between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

“Pharmaceutically acceptable carriers” refers to any diluents, excipients, or carriers that may be used in the compositions disclosed herein. In some embodiments, a pharmaceutically acceptable carrier comprises, or consists essentially of, or yet further consists of a nanoparticle, such as an polymeric nanoparticle carrier or an lipid nanoparticle (LNP). Additionally or alternatively, pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They can be selected with respect to the intended form of administration, that is, oral tablets, capsules, elixirs, syrups and the like, and consistent with conventional pharmaceutical practices.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form. A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The terms “polypeptide,” “peptide,” and “protein” are used interchangeably herein to refer to a polymer of amino acid residues. The terms apply to naturally occurring amino acid polymers as well as amino acid polymers in which one or more amino acid residues are a non-naturally occurring amino acid, e.g., an amino acid analog. The terms encompass amino acid chains of any length, including full length proteins, wherein the amino acid residues are linked by covalent peptide bonds.

The terms “pyruvate kinase M2,” and “PKM2” are used interchangeably and refer to a protein involved in glycolysis. The encoded protein is a pyruvate kinase that catalyzes the transfer of a phosphoryl group from phosphoenolpyruvate to ADP, generating ATP and pyruvate. In some embodiments, the PKM2 is a human PKM2. Non-limiting exemplary sequences of this protein or the underlying gene can be found under Gene Cards ID: GC15M072199, HGNC: 9021, NCBI Entrez Gene: 5315, Ensembl: ENSG00000067225, OMIM®: 179050, or UniProtKB/Swiss-Prot: P14618, each of which is incorporated by reference herein in its entirety.

As used herein, the term “PKM2 activator” refers to an agent that increases the level of pyruvate kinase activity of PKM2, such as from the state of inactive monomeric or dimeric form or maintains or increases the activity of active tetrameric form of PKM2 (e.g., in the presence of an endogenous inhibitor.

As used herein, the term “reduce” means to alter negatively by at least about 5%, including, but not limited to, alter negatively by about 5%, by about 10%, by about 25%, by about 30%, by about 50%, by about 75%, or by about 100%.

As used herein, “regulatory sequence” of a nucleic acid molecule means a cis-acting nucleotide sequence that influences expression, positively or negatively, of an operably linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration, gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Polymerase II and III are examples of promoters. A polymerase II or “pol II” promoter catalyzes the transcription of DNA to synthesize precursors of mRNA, and most shRNA and microRNA. Examples of pol II promoters are known in the art and include without limitation, the phosphoglycerate kinase (“PGK”) promoter; EF1-alpha; CMV (minimal cytomegalovirus promoter); and LTRs from retroviral and lentiviral vectors. In some embodiments, the promoter is a constitutive promoter. As used herein, the term “constitutive promoter” refers to a promoter that allows for continual transcription of the coding sequence or gene under its control in all or most tissues of a subject at all or most developing stages. Non-limiting examples of the constitutive promoters include a CMV promoter, a simian virus 40 (SV40) promoter, a polyubiquitin C (UBC) promoter, an EF1-alpha promoter, a PGK promoter and a CAG promoter. In some embodiments, the promoter is a conditional promoter, which allows for continual transcription of the coding sequence or gene under certain conditions. In further embodiments, the conditional promoter is an immune cell specific promoter, which allows for continual transcription of the coding sequence or gene in an immune cell. Non-limiting examples of the immune cell specific promoters include a promoter of a B29 gene promoter, a CD14 gene promoter, a CD43 gene promoter, a CD45 gene promoter, a CD68 gene promoter, a IFN-β gene promoter, a WASP gene promoter, a T-cell receptor β-chain gene promoter, a V9 γ (TRGV9) gene promoter, a V2 δ (TRDV2) gene promoter, and the like.

Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, but are not limited to, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, the term “sample” refers to clinical samples obtained from a subject. In certain embodiments, a sample is obtained from a biological source (i.e., a “biological sample”), such as tissue, bodily fluid, or microorganisms collected from a subject. Sample sources include, but are not limited to, mucus, sputum, bronchial alveolar lavage (BAL), bronchial wash (BW), whole blood, bodily fluids, cerebrospinal fluid (CSF), urine, plasma, serum, or tissue.

As used herein, the term “secreted” in reference to a polypeptide means a polypeptide that is released from a cell via the secretory pathway through the endoplasmic reticulum, Golgi apparatus, and as a vesicle that transiently fuses at the cell plasma membrane, releasing the proteins outside of the cell. Small molecules, such as drugs, can also be secreted by diffusion through the membrane to the outside of cell.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject,” “individual,” or “patient” are used interchangeably and refer to an individual organism, a vertebrate, or a mammal and may include humans, non-human primates, rodents, and the like (e.g., which is to be the recipient of a particular treatment, or from whom cells are harvested). In certain embodiments, the individual, patient or subject is a human.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods. As used herein, production by recombinant means by using recombinant DNA methods means the use of the well-known methods of molecular biology for expressing proteins encoded by cloned DNA.

As used herein, the term “T-cell” includes naïve T cells, CD4+ T cells, CD8+ T cells, memory T cells (including central memory T cells, stem-cell-like memory T cells (or stem-like memory T cells), and two types of effector memory T cells: e.g., T_{EM} cells and TEMRA cells), activated T cells, anergic T cells, tolerant T cells, chimeric B cells, Regulatory T cells (also known as suppressor T cells), Natural killer T cells, Mucosal associated invariant T cells, and γδ T cells, and antigen-specific T cells.

As used herein, “T cell receptor” or “TCR”, is a protein complex found on the surface of T cells, that is responsible for recognizing fragments of antigen as peptides bound to major histocompatibility complex molecules. TCR is composed of two disulfide-linked protein chains. Cells expressing a TCR containing the highly variable alpha (α) and beta (β) chains are referred to as αβ T cells. Cells expressing an alternate TCR, formed by variable gamma (γ) and delta (δ) chains, are referred to as γδ T cells. When the TCR engages with antigenic peptide and MHC (peptide/MHC), the T lymphocyte is activated through signal transduction, that is, a series of biochemical events mediated by associated enzymes, co-receptors, specialized adaptor molecules, and activated or released transcription factors. In some embodiments, the TCR is a native T cell receptor that is endogenous to the immune cells. In some embodiments, the TCR is an artificial receptor that mimics native TCR function, i.e., recognizing peptide antigens of key intracellular proteins in the context of MHC on the cell surface.

The terms “T-Cell-Specific Transcription Factor 1,” “TCF1” and “TCF-1” are used interchangeably and refer to a member of the T-cell factor/lymphoid enhancer-binding factor family of high mobility group (HMG) box transcriptional activators. This gene is expressed predominantly in T-cells and plays a critical role in natural killer cell and innate lymphoid cell development. The encoded protein forms a complex with beta-catenin and activates transcription through a Wnt/beta-catenin signaling pathway. In some embodiments, the TCF-1 is a human TCF-1. Non-limiting exemplary sequences of this protein or the underlying gene can be found under Gene Cards ID: GC05P134114, HGNC: 11639, NCBI Entrez Gene: 6932, Ensembl: ENSG00000081059, OMIM®: 189908, or UniProtKB/Swiss-Prot: P36402, each of which is incorporated by reference herein in its entirety.

The terms “Tumor Necrosis Factor α,” “TNFα” and “TNF-α” are used interchangeably and refer to a multifunctional proinflammatory cytokine that belongs to the tumor necrosis factor (TNF) superfamily. This cytokine is mainly secreted by macrophages. It can bind to, and thus functions through its receptors TNFRSF1A/TNFR1 and TNFRSF1B/TNFBR. This cytokine is involved in the regulation of a wide spectrum of biological processes including cell proliferation, differentiation, apoptosis, lipid metabolism, and coagulation. In some embodiments, the TNFα is a human TNFα. Non-limiting exemplary sequences of this protein or the underlying gene can be found under Gene Cards ID: GC06P061170, HGNC: 11892, NCBI Entrez Gene: 7124, Ensembl: ENSG00000232810, OMIM®: 191160, or UniProtKB/Swiss-Prot: P01375, each of which is incorporated by reference herein in its entirety.

As used herein “tumor-infiltrating” immune cells refer to immune cells that have left the bloodstream and migrated into a tumor.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. Therapeutic effects of treatment include, without limitation, inhibiting recurrence of disease, alleviation of symptoms, diminishment of any direct or indirect pathological consequences of the disease, preventing metastases, decreasing the rate of disease progression, amelioration or palliation of the disease state, and remission or improved prognosis. By “treating a cancer” is meant that the symptoms associated with the cancer are, e.g., alleviated, reduced, cured, or placed in a state of remission.

It is also to be appreciated that the various modes of treatment of diseases as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

### Adoptive Cell Therapy (ACT)

CAR T cell therapy has gained momentum after several promising clinical trials for the treatment of B-cell neoplasms and the FDA approval of a CD19 targeted CAR T cell for treatment of B cell acute lymphoid leukemia (Sadelain et al., Nature 545:423-431 (2017); Yu et al., J Hematol Oncol. 10:78 (2017); Kakarla and Gottschalk, Cancer J. 20:151-155 (2014); Wang et al., J Hematol Oncol. 10:53 (2017)). CAR T cell therapy involves isolating a patient's own T cells, engineering them to express a CAR, and reinfusing the engineered T cells back into the patient. The CAR contains an extracellular single-chain variable fragment (scFv), a transmembrane domain, and an intracellular signaling domain. Surface expression of a tumor-targeted scFv on the T cell results in tumor antigen-directed T cell activation and specific tumor killing via its signaling domain. However, many patients with hematologic cancers treated with CAR T cell therapy relapse with antigen loss variants as a result of tumor editing (Wang et al., J Hematol Oncol. 10:53 (2017)). Furthermore, translation of CAR T cell therapy to solid tumors has been difficult due to the immunosuppressive tumor environment (TME) (Yu et al., J Hematol Oncol. 10:78 (2017); Kakarla and Gottschalk, Cancer J. 20:151-155 (2014)).

The TME consists of physical barriers, such as surrounding fibroblasts and extracellular matrix proteins, which make tumors less accessible to the T cells. Beyond this dense stromal network, T cell can encounter a number of inhibitory immune cells such as regulatory T cells, myeloid suppressor cells and tumor associated macrophages, as well an upregulation of immune checkpoint molecules, rendering the cytotoxic T cells inactive (Newick et al., Annu Rev Med. 1-14 (2016)). These immune checkpoints normally play a role in self recognition to prevent autoimmune responses, but are upregulated by many cancers to suppress immune cells (Topalian et al., Cancer Cell 27:451-461 (2015) and Postow et al., J Clin Oncol. 33:1974-1982 (2015)).

When the immune checkpoint proteins CTLA-4 and PD-1 receptors are expressed on the T cell surface, they function through distinct mechanisms to downregulate T cell activity to prevent autoimmunity and maintain immunological homeostasis (Postow et al., J Clin Oncol. 33:1974-1982 (2015)). Although immune checkpoint blockade therapies have been successful in treating patients with various cancers, patient response rate is variable (Matlung et al., Immunol Rev. 276:145-164 (2017); Rizvi et al., Science 348:124-128 (2015); Chao et al., Cell 24:225-232 (2011)).

The importance of sustained T cell activity for ACT has generated a great deal of interest in methods that increase the effective size of therapeutic T cell populations and prolong their anti-tumor functionality. Patients are typically subjected to conditioning chemotherapy or ablative radiation prior to infusion in order to reduce competition between transferred T cells and endogenous lymphocytes for homeostatic cytokines. Although these approaches significantly increase engraftment efficiency, a large fraction of infused T cells still perish within days (J. N. Blattman et al., J Exp Med 195, 657-664 (2002); C. Berger et al., J Clin Invest 118, 294-305 (2008)), and strategies for mitigating this death are poorly developed. Maintaining functional persistence after engraftment is also of critical importance. Provided herein are compositions comprising engineered immune cells that overexpress GLUT5 and uses thereof that address these issues.

In addition, provided herein are compositions comprising heterologous nucleic acids encoding GLUT5, vectors comprising heterologous nucleic acids encoding GLUT5, or engineered immune cells expressing heterologous nucleic acids encoding GLUT5 and methods of using such compositions for the manufacture of an engineered immune cell. Without wishing to be bound by theory, ectopic expression of GLUT5 renders T cells less vulnerable to exhaustion and more able to kill tumor cells. Consequently, T cells overexpressing GLUT5 display enhanced anti-tumor activity in both T cell receptor (TCR) and CAR driven models of ACT.

In some embodiments, the engineered immune cells provided herein express a T-cell receptor (TCR) or other cell-surface ligand that binds to a target antigen, such as a tumor antigen or viral protein. In some embodiments, the T cell receptor is a wild-type or native T-cell receptor. In some embodiments, the TCR is an engineered receptor or a non-native receptor. In some embodiments, the engineered receptor is an engineered TCR (eTCR). In some embodiments, the engineered receptor is a chimeric antibody TCR (caTCR). In some embodiments, the engineered receptor is a chimeric antigen receptor (CAR).

In exemplary embodiments, the engineered immune cells provided herein express a native receptor, a non-native receptor, or an engineered receptor (e.g., a CAR, caTCR, or eTCR) or other cell-surface ligand that binds to a tumor antigen. In some embodiments, the engineered immune cells provided herein express a native receptor, a non-native receptor, or an engineered receptor (e.g., a CAR, caTCR, or eTCR) or other cell-surface ligand that binds to a tumor antigen presented in the context of an MHC molecule. In some embodiments, the engineered immune cells provided herein express a native receptor, a non-native receptor or an engineered receptor (e.g., a CAR, caTCR, or eTCR) or other cell-surface ligand that binds to a tumor antigen presented in the context of an HLA-A2 molecule. In exemplary embodiments, the engineered immune cells provided herein express a native receptor, a non-native receptor or an engineered receptor (e.g., a CAR, caTCR, or eTCR) or other cell-surface ligand that binds to a tumor antigen. Examples of tumor antigens bound by the native receptor, non-native receptor or engineered receptor (e.g., a CAR, caTCR, or eTCR) or other cell-surface ligand include, but is not limited to, disialoganglioside GD2 (GD2), mucin 1 (MUC1), prostate-specific membrane antigen (PSMA), human epidermal growth factor receptor 2 (Her2), mucin 16 (MUC16), melanoma-associated antigen 1 (MAGE-A1), carbonic anhydrase 9 (CAIX), b-lymphocyte surface antigen CD19 (CD19), prominin-1 (CD133), CD33 antigen (CD33), CD38 antigen (CD38), neural cell adhesion molecule (CD56), interleukin-3 receptor (CD123), and b-lymphocyte antigen CD20 (CD20). Exemplary engineered receptors that bind to CD19 are described in International Publication No. WO2017070608, which is incorporated by reference in its entirety.

In one aspect, the metabolic features of T_{eff} cells are engineered to adapt in glucose-limited conditions and enhance their antitumor effector functions so as to confront the challenge of interrogating metabolic flux in vivo. In further embodiments, T_{eff} cells are engineered to utilize fructose as an alternative to glucose for their major carbon source. Fructose has the same molecular formula as glucose, but most cells, including cancer cells, consume it slowly due to their low expression of fructose transporters, such as GLUT5. The present disclosure demonstrates that GLUT5 overexpression (GLUT5-OV) can readily convert cancer cells into rapid consumers of fructose through glycolysis, and without wishing to be bound by the theory, T_{eff} cells with GLUT5-OV readily utilize fructose to maintain the level of glycolytic intermediates and reduce defects in their immune responses in glucose-deprived tumor microenvironments when exogenous fructose is provided. The metabolic states and effector functions of GLUT5-OV T_{eff} cells are determined with modulation of glucose and fructose levels in vitro. A strategy is further developed to increase the fructose level in the tumor microenvironment as well as to apply isotopic tracing and HP MRI methods to interrogate metabolism and T-cell efficacy.

In some embodiments, the disclosure herein is to address this critical energetic need of T cells by developing a system to overexpress GLUT5 or another fructose transporter in effector T cells. In the presence of levels of extracellular fructose inadequate to fuel glycolysis in tumor cells, T cells would be able to meet all their energetic demands. This engineer's approach to selectively fueling T cells would require only the transient addition of a non-toxic endogenous dietary component, fructose, and could advance the understanding of in vivo cancer biology. As it would be understood by one of skill in the art, such T cells can be substituted by other immune cells cytotoxic to tumor cells or capable of enhancing the cytotoxicity to tumor cells.

As illustrated in the Example, a newly developed T-cell exhaustion model is used; fructose is traced and imaged to optimize overexpression of GLUT5; and an in vivo investigation is performed. The impact of sugar use in these T cells are verified as well as the effect on energy metabolism and T-cell function. This approach provides a novel strategy for using diet to “turbo-charge” T cells in vivo that can readily be translated to the clinic.

Without wishing to be bound by the theory, enhancing the T cells' ability to metabolize fructose renders them less vulnerable to exhaustion and more able to kill tumor cells. This metabolic switch can be followed using LC/MS and hyperpolarized MRI isotope tracing in vitro and in vivo. Taken together, this rigorous approach as disclosed herein, supported by both extensive literature and the data shown herein, addresses a significant need in the field of immunotherapy and in the study of immunometabolism.

### Overview of Fructose Metabolism in Immune Cells

Fructose Metabolism: Dietary fructose consumption, which has increased >100-fold over the past two centuries, now accounts for ˜10% of total caloric intake in the United States (Vos et al. Medscape J Med 10, 160 (2008); Bray et al. Am J Clin Nutr 79, 537-543 (2004); and Marriott et al., J Nutr 139, 1228S-1235S (2009)). As fructose is more palatable but less satiating than glucose, overconsumption often leads to the development of obesity and metabolic syndrome (Macdonald et al. Eur J Nutr 55, 17-23 (2016)). For nearly 100 years it has been known that most cancer cells exhibit a high glycolytic rate, even in the presence of oxygen (Warburg Effect). Recently, multiple studies have demonstrated that cancer cells are able to utilize fructose as an additional fuel for their proliferation and metastasis. For example, pancreatic cancer cells have been shown to increase flux through the non-oxidative pentose phosphate pathway in fructose-rich conditions, which leads to the preferential use of fructose over glucose for nucleotide synthesis (Liu et al. Cancer research 70, 6368-6376 (2010)). Breast cancer and colon cancer cells with elevated levels of aldolase-B, a key enzyme in fructose metabolism, were shown to metastasize at high levels to liver under a high-fructose diet (Bu et al. Cell metabolism 27, 1-41 (2018)). GLUT5, a potent transporter of fructose, has been shown to be upregulated in some patients with leukemia or lung adenocarcinoma, which facilitates the fructose use of cancer cells in glucose-limited conditions (Chen et al. Cancer cell 30, 779-791 (2016); Weng et al. Cell Death Discov 4, 38 (2018)). Without wishing to be bound by the theory, the metabolic flexibility of cancer cells, which exploit any available carbon source to adapt and proliferate in diverse environments, can be one reason why the increased uptake of fructose in the diet has been associated with cancer progression. In general, though, the uptake of fructose in cancers is slow and carbons derived from fructose typically are unable to populate glycolytic intermediates at near the level of glucose.

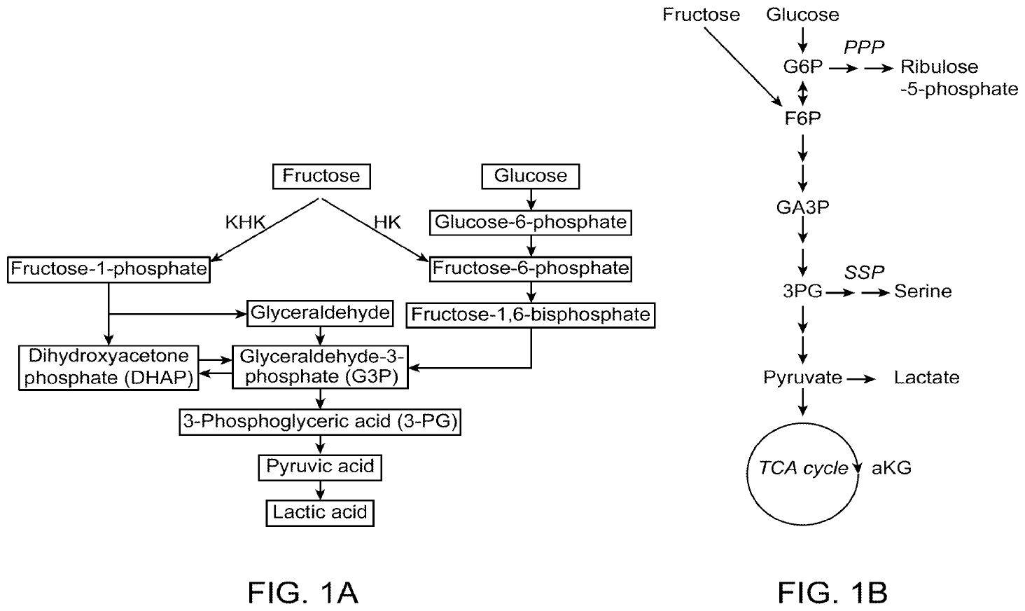

In normal physiology, fructose is predominantly metabolized in the liver and small intestine, utilizing rapid transport via the insulin-independent transporters GLUT2 and GLUT5 (Douard & Ferraris. American journal of physiology Endocrinology and metabolism 295, E227-237 (2008); Jang et al. Cell Metab 27, 351-361 e353 (2018); Goncalves et al. Science 363, 1345-1349 (2019)). It is subsequently converted to fructose-1-phosphate by the enzyme ketohexokinase (KHK) and can participate in further biochemical transformations in glycolysis (FIG. 1). Fructose can also be converted directly to fructose-6-phosphate by hexokinase, though at a rate significantly slower than that of KHK. Ultimately, transport and these first enzymatic conversions drive the metabolism of fructose and, in many instances, gluconeogenesis. While fructose has been implicated as a potential carbon source for cancer cells, the underlying mechanism remains unclear, especially as most cancer cells do not have the ability to drive glycolytic flux using fructose.

Recent work has suggested glycolytic production of lactate is required for T-cell function, making reduced glucose a fundamental limitation for effective immunotherapy. Fructose, a monosaccharide that is not transported by the predominant glucose transporter (GLUT1), is typically is taken up by the liver, kidneys and small intestine via the insulin independent transporters GLUT2 and GLUT5. In these tissues, the enzyme ketohexokinase (KHK) is expressed at high levels, facilitating the formation of fructose-1-phosphate and in most cases gluconeogenesis. Interestingly, most tumors take up fructose slowly, due to low levels of GLUT2/5 expression, high Kₘ of fructose for GLUT1 and lack of KHK. A mechanism is identified herein whereby fructose is metabolized through the serine synthesis pathway (SSP), generating insignificant lactate via glycolysis. Without wishing to be bound by the theory, this can be modulated by the fructose transporter, titrating carbons from the SSP to glycolysis and deriving lactate from fructose equivalent to glucose.

T-cell metabolism in the tumor microenvironment: Immunotherapy has surged as a powerful therapeutic option for cancer, but its effect has only been demonstrated in a subset of cancers. A deeper understanding of the key mechanisms behind immune cells losing their antitumor functions is necessary for the development of more effective therapeutic options. Growing evidence suggests metabolic conditions in the tumor microenvironment are unfavorable for immune cells and dampen their antitumor functions (Kouidhi et al. Front Immunol 8, 270 (2017); and Martinez-Outschoorn et al. Nat Rev Clin Oncol 14, 11-31 (2017)). In some embodiments, tumor cells rapidly reduce levels of extracellular glucose, leading to a low steady state concentration (e.g. 1 mM). For example, as a consequence of multiple genetic mutations in signaling pathways, including PI3K, cancer cells often exhibit increased glucose metabolism, which leads to glucose-deprived tumor microenvironments (Hirayama et al. Cancer Res 69, 4918-4925 (2009); and Urasaki et al. PLoS One 7, e36775 (2012)). Unfortunately, immune cells, particularly effector T (T_{eff}) cells, are heavily dependent on glycolysis for their immune response and may be affected by the tumor microenvironment (O'Sullivan et al. Nature reviews. Immunology 19, 324-335 (2019)); T-cell activation is accompanied by the increased expression of the glucose transporter GLUT1 and the glycolytic enzymes, including LDHA (Chang et al. Cell 162, 1229-1241 (2015); MacIver et al. Annu RevImmunol 31, 259-283 (2013); Geltink et al. Annu Rev Immunol 36, 461-488 (2018)). The enhanced glycolysis in T cells not only provides biosynthetic and energetic precursors, but also supports interferon gamma (IFN-γ) production through epigenetic reprogramming (Chang et al. Cell 153, 1239-1251 (2013); Peng et al. Science 354, 481-484 (2016)). Recent studies have shown that the decreased level of glycolytic intermediates in T_{eff} cells leads to defects in their Ca²⁺ signaling (Ho et al. Cell 162, 1217-1228 (2015)) and N-linked glycosylation (Song et al. Nature 562, 423-428 (2018)), both of which are critical for their immune responses. It has been consistently observed that the high glycolytic rates of tumors from melanoma and lung cancer patients correlate with poor effector functions of tumor-infiltrating T cells and resistance to adoptive T-cell therapy (Cascone et al. Cell Metab 27, 977-987 e974 (2018). Recent work has also implicated increased enolase active tumor infiltrating lymphocytes (TILs) in the setting of checkpoint blockade, further linking glycolytically flux to potential enhancement of immunotherapy (Gemta et al. Sci Immunol 4 (2019); and Zappasodi et al. Cancer cell 33, 581-598 (2018)). Thus, without wishing to be bound by the theory, if the availability of nutrients to feed T cells can be increased in this deprived condition, glycolytic flux can be restored.

Limitations of current models in exploring immunometabolism: Conventional study of immune-mediated responses to tumors and reinvigoration by checkpoint blockade has largely relied on the use of in vivo models in which tumor-infiltrating T cells are extracted from subcutaneous tumors and evaluated ex vivo. However, these model systems have numerous limitations. First, the metabolism of cells is altered within minutes of being removed from their native environment, limiting the interpretability of T cells that are extracted from tumors in a multi-step process over two to three hours. Second, solely evaluating the metabolic behavior of surviving cells within a tumor ignores metabolic susceptibilities that might prevent the effective accumulation of intratumoral T cells. To overcome these deficiencies, developed herein is a novel in vitro system in which T cells chronically encounter tumor cells in an antigen-specific fashion. This approach offers a number of advantages. First, with great sensitivity, both control nutrient availability and easily introduce uniformly labeled tracers can be used to track T-cell metabolism. Second, T cells can be rapidly isolated, thereby avoiding any metabolic changes that might occur during the process of T-cell extraction from tumors. Finally, metabolic behavior can be traced during the development of T-cell dysfunction, enabling identification of metabolic features that might drive the elimination of T cells from tumors over time. Overcoming this limitation is necessary to explore metabolic modulation in T cells and their impact on tumor biology.

Limitations of current methods for measuring metabolic flux in mass limited systems: The difficulty of acquiring large numbers of primary T cells presents a challenge for metabolic study, especially in the case of genetically engineered T cells. Typically, metabolism is assessed in two ways, first in terms of the steady state distribution of metabolites, or “metabolomics,” and, second, in terms of rates of conversion of substrates to products in a pathway, or “flux”. Given the interest in sources of metabolites and the rates at which metabolites are made, flux measurements have become a staple of cancer metabolism research. Provocative studies have demonstrated that the source of some metabolites—effectively, the flux from a nutrient to the metabolite of interest—is dramatically different than previously expected given the literature and metabolomics studies. See, Viale et al. Nature 514, 628-632 (2014); Mashimo et al. Cell 159, 1603-1614 (2014); and Fan et al. Nature 510, 298-302 (2014). Recently, numerous methods have been developed to access fluxes non-invasively in living systems (e.g., NMR and fluorescence) as well as by destructive methods (e.g., GC and LC/MS). To monitor a reaction, these approaches exploit either isotopically enriched metabolic substrates, following them as they are metabolized, or specific fluorescent substrates. Unfortunately, the approaches suffer from sensitivity (e.g., NMR requiring 10⁸ cells to make a measurement non-invasively or LC/MS requiring 10⁶ cells destructively) and difficulty in measuring multiple metabolic fluxes robustly with high-throughput (e.g., fluorescence requiring the development of many strategies to measure multiple metabolites). Given the interest in studying T-cell metabolism, both realistic models of T-cell exhaustion and methods to assess them are needed to better understand immunotherapy and immunometabolism.

Hyperpolarized MRI and isotope tracing to interrogate in vivo flux: Notwithstanding the great interest in understanding metabolic fluxes, interrogating them in vivo is extremely challenging. Hyperpolarized MRI can address this challenge by creating MRI probes, which can take part in these metabolic reactions (Qu et al. Acad Radiol 18, 932-939 (2011); Gallagher et al. Magn Reson Med 66, 18-23 (2011)). Utilizing this approach, the NMR signal of a molecule was increased by the Applicant by nearly 10⁵-fold and it was followed through enzymatic reactions in seconds (Koelsch et al. Analyst 138, 1011-1014 (2013); Hu et al. Cell Metab 14, 131-142 (2011); and Keshari et al. Cancer research 73, 529-538 (2013)). This approach was used by the Applicant to develop multiple metabolic probes for use in vivo, including HP pyruvate (to trace changes in glycolysis and the TCA cycle), HP glutamine (to measures glutaminolysis and production of the oncometabolite 2-HG) as well as HP dehydroascorbate (to assess in vivo redox capacity and oxidative stress). See Di Gialleonardo et al. Cancer Res 77, 3113-3120 (2017); Salamanca-Cardona et al. Cell Metab 26, 830-841 e833 (2017); Keshari et al. Proc Natl Acad Sci USA 108, 18606-18611 (2011).

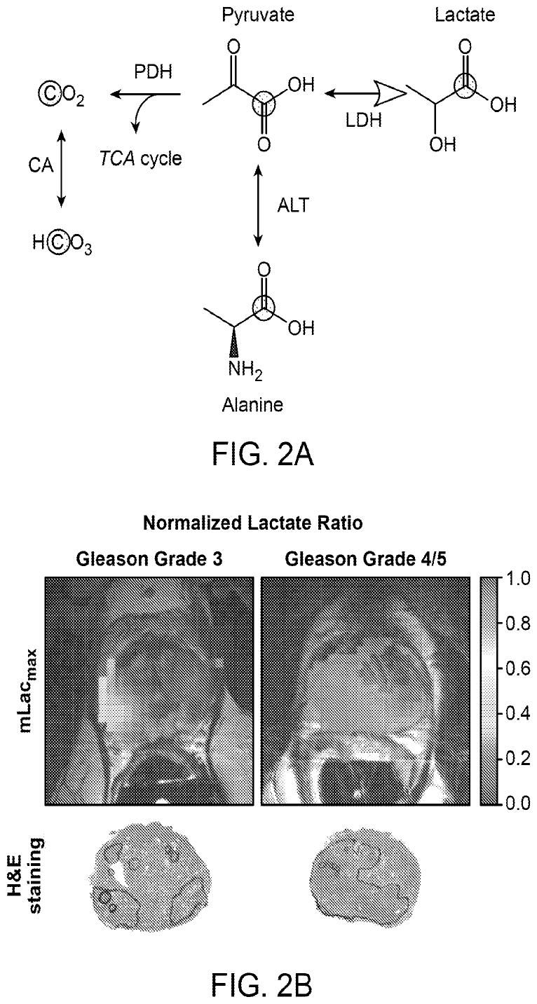

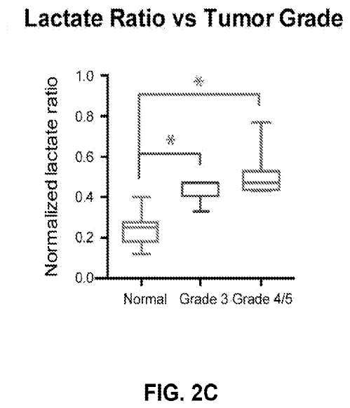

These endogenous nutrients represent a class of imaging agents that can be utilized in vivo at high concentrations and rapidly cleared using normal physiologic processes, making them easily applied in the setting of repeat measurements of in vivo metabolic flux. The metabolism of a number of these molecules was confirmed by the Applicant in vivo and in doing so rapid bolus methods were developed with LC/MS and NMR (Salamanca-Cardona et al. Cell Metab 26, 830-841 e833 (2017); Cho et al. ACS Chem Biol 14(4):665-673 (2019)), which is used extensively in the Examples as described herein. Furthermore, the Applicant has acquired first human breast cancer as well as prostate cancer and brain data using HP pyruvate, making this approach readily applicable to imaging humans (FIG. 2).

### GLUT5

As used herein, the terms “glucose transporter 5,” “GLUT5,” “Solute Carrier Family 2 Member 5” and “SLC2A5” refer to a fructose transporter responsible for fructose uptake by the small intestine, or a gene encoding the fructose transporter. The encoded protein also is necessary for the increase in blood pressure due to high dietary fructose consumption. Non-limiting exemplary sequences of this protein or the underlying gene may be found under Gene Cards ID: GC01M009036 (retrieved from www.genecards.org/cgi-bin/carddisp.pl?gene=SLC2A5), HGNC: 1010 (www.genenames.org/data/gene-symbol-report/#!/hgnc_id/11010), NCBI Entrez Gene: 6518 (retrieved from www.ncbi.nlm.nih.gov/gene/6518), Ensembl: ENSG00000142583 (retrieved from uswest.ensembl.org/Homo_sapiens/Gene/Summary?g=ENSG00000142583;r=1:9035106-9088478), OMIM®: 138230 (retrieved from omim.org/entry/138230), or UniProtKB/Swiss-Prot: P22732 (retrieved from www.uniprot.org/uniprot/P22732), which are incorporated by reference herein.

Exemplary amino acid sequences of human GLUT5 are set forth below:

Solute carrier family 2, facilitated glucose
transporter member 5 isoform 1
(SEQ ID NO: 1)
MEQQDQSMKEGRLTLVLALATLIAAFGSSFQYGYNVAAVNSPALLMQQFY
NETYYGRTGEFMEDFPLTLLWSVTVSMFPFGGFIGSLLVGPLVNKFGRKG
ALLFNNIFSIVPAILMGCSRVATSFELIIISRLLVGICAGVSSNVVPMYL
GELAPKNLRGALGVVPQLFITVGILVAQIFGLRNLLANVDGWPILLGLTG
VPAALQLLLLPFFPESPRYLLIQKKDEAAAKKALQTLRGWDSVDREVAEI
RQEDEAEKAAGFISVLKLFRMRSLRWQLLSIIVLMGGQQLSGVNAIYYYA
DQIYLSAGVPEEHVQYVTAGTGAVNVVMTFCAVFVVELLGRRLLLLLGFS
ICLIACCVLTAALALQDTVSWMPYISIVCVISYVIGHALGPSPIPALLIT
EIFLQSSRPSAFMVGGSVHWLSNFTVGLIFPFIQEGLGPYSFIVFAVICL
LTTIYIFLIVPETKAKTFIEINQIFTKMNKVSEVYPEKEELKELPPVTSE
Q;
or
Solute carrier family 2, facilitated glucose
transporter member 5 isoform 2
(SEQ ID NO: 2)
MEQQDQSMKEGRLTLVLALATLIAAFGSSFQYGYNVAAVNSPALLMQQFY
NETYYGRTGEFMEDFPLTLLWSVTVSMFPFGGFIGSLLVGPLVNKFGRKG
ALLFNNIFSIVPAILMGCSRVATSFELIIISRLLVGICAGVSSNVVPMYL
GELAPKNLRGALGVVPQLFITVGILVAQIFGLRNLLANVDGEFRTSREHP
HPFTTTLGPLLVFQSHHHRTGLSADWSLLTGWMSLGGPSCPEPT

In some embodiments, the engineered immune cells express a heterologous amino acid sequence that is at least 80%, 85%, 90%, 95%, 96%, 97%, 98%, or 99% identical to SEQ ID NO: 1, SEQ ID NO: 2, or a biological equivalent thereof. In further embodiments, the biological equivalent of SEQ ID NO: 1 or SEQ ID NO: 2 comprises one or more conservative amino acid substitutions relative to SEQ ID NO: 1 or SEQ ID NO: 2, respectively. Additionally or alternatively, in some embodiments, the biological equivalent transports fructose substantially similar to or significantly more efficiently compared to the protein of SEQ ID NO: 1 or 2.

Exemplary nucleic acid sequences of human GLUT5 are set forth below:

(SEQ ID NO: 7)
atggagcaacaggatcagagcatgaaggaagggaggctgacgcttgtgct
tgccctggcaaccctgatagctgcctttgggtcatccttccagtatgggt
acaacgtggctgctgtcaactccccagcactgctcatgcaacaattttac
aatgagacttactatggtaggaccggtgaattcatggaagacttcccctt
gacgttgctgtggtctgtaaccgtgtccatgtttccatttggagggttta
tcggatccctcctggtcggccccttggtgaataaatttggcagaaaaggg
gccttgctgttcaacaacatattttctatcgtgcctgcgatcttaatggg
atgcagcagagtcgccacatcatttgagcttatcattatttccagacttt
tggtgggaatatgtgcaggtgtatcttccaacgtggtccccatgtactta
ggggagctggcccctaaaaacctgcggggggctctcggggtggtgcccca
gctcttcatcactgttggcatccttgtggcccagatctttggtcttcgga
atctccttgcaaacgtagatggtgagttcaggacatctcgggagcacccc
caccccttcaccactacccttggccccctccttgtgttccaaagccacca
ccacaggacaggactttctgcagactggtctcttctaacaggctggatgt
ccttggggggcccatcctgtcccgagccaacatag;
(SEQ ID NO: 8)
atggagcaacaggatcagagcatgaaggaagggaggctgacgcttgtgct
tgccctggcaaccctgatagctgcctttgggtcatccttccagtatgggt
acaacgtggctgctgtcaactccccagcactgctcatgcaacaattttac
aatgagacttactatggtaggaccggtgaattcatggaagacttcccctt
gacgttgctgtggtctgtaaccgtgtccatgtttccatttggagggttta
tcggatccctcctggtcggccccttggtgaataaatttggcagaaaaggg
gccttgctgttcaacaacatattttctatcgtgcctgcgatcttaatggg
atgcagcagagtcgccacatcatttgagcttatcattatttccagacttt
tggtgggaatatgtgcaggtgtatcttccaacgtggtccccatgtactta
ggggagctggcccctaaaaacctgcggggggctctcggggtggtgcccca
gctcttcatcactgttggcatccttgtggcccagatctttggtcttcgga
atctccttgcaaacgtagatggctggccgatcctgctggggctgaccggg
gtccccgcggcgctgcagctccttctgctgcccttcttccccgagagccc
caggtacctgctgattcagaagaaagacgaagcggccgccaagaaagccc
tacagacgctgcgcggctgggactctgtggacagggaggtggccgagatc
cggcaggaggatgaggcagagaaggccgcgggcttcatctccgtgctgaa
gctgttccggatgcgctcgctgcgctggcagctgctgtccatcatcgtcc
tcatgggcggccagcagctgtcgggcgtcaacgctatctactactacgcg
gaccagatctacctgagcgccggcgtgccggaggagcacgtgcagtacgt
gacggccggcaccggggccgtgaacgtggtcatgaccttctgcgccgtgt
tcgtggtggagctcctgggtcggaggctgctgctgctgctgggcttctcc
atctgcctcatagcctgctgcgtgctcactgcagctctggcactgcagga
cacagtgtcctggatgccatacatcagcatcgtctgtgtcatctcctacg
tcataggacatgccctcgggcccagtcccatacccgcgctgctcatcact
gagatcttcctgcagtcctctcggccatctgccttcatggtggggggcag
tgtgcactggctctccaacttcaccgtgggcttgatcttcccgttcatcc
aggagggcctcggcccgtacagcttcattgtcttcgccgtgatctgcctc
ctcaccaccatctacatcttcttgattgtcccggagaccaaggccaagac
gttcatagagatcaaccagattttcaccaagatgaataaggtgtctgaag
tgtacccggaaaaggaggaactgaaagagcttccacctgtcacttcggaa
cagtga;
or
(SEQ ID NO: 9)
atggagcaacaggatcagagcatgaaggaagggaggctgacgcttgtgct
tgccctggcaaccctgatagctgcctttgggtcatccttccagtatgggt
acaacgtggctgctgtcaactccccagcactgctcatgcaacaattttac
aatgagacttactatggtaggaccggtgaattcatggaagacttcccctt
gacgttgctgtggtctgtaaccgtgtccatgtttccatttggagggttta
tcggatccctcctggtcggccccttggtgaataaatttggcagaaaaggg
gccttgctgttcaacaacatattttctatcgtgcctgcgatcttaatggg
atgcagcagagtcgccacatcatttgagcttatcattatttccagacttt
tggtgggaatatgtgcaggtgtatcttccaacgtggtccccatgtactta
ggggagctggcccctaaaaacctgcggggggctctcggggtggtgcccca
gctcttcatcactgttggcatccttgtggcccagatctttggtcttcgga
atctccttgcaaacgtagatggctggccgatcctgctggggctgaccggg
gtccccgcggcgctgcagctccttctgctgcccttcttccccgagagccc
caggtacctgctgattcagaagaaagacgaagcggccgccaagaaagccc
tacagacgctgcgcggctgggactctgtggacagggaggtggccgagatc
cggcaggaggatgaggcagagaaggccgcgggcttcatctccgtgctgaa
gctgttccggatgcgctcgctgcgctggcagctgctgtccatcatcgtcc
tcatgggcggccagcagctgtcgggcgtcaacgctatctactactacgcg
gaccagatctacctgagcgccggcgtgccggaggagcacgtgcagtacgt
gacggccggcaccggggccgtgaacgtggtcatgaccttctgcgccgtgt
tcgtggtggagctcctgggtcggaggctgctgctgctgctgggcttctcc
atctgcctcatagcctgctgcgtgctcactgcagctctggcactgcagga
cacagtgtcctggatgccatacatcagcatcgtctgtgtcatctcctacg
tcataggacatgccctcgggcccagtcccatacccgcgctgctcatcact
gagatcttcctgcagtcctctcggccatctgccttcatggtggggggcag
tgtgcactggctctccaacttcaccgtgggcttgatcttcccgttcatcc
aggagggcctcggcccgtacagcttcattgtcttcgccgtgatctgcctc
ctcaccaccatctacatcttcttgattgtcccggagaccaaggccaagac
gttcatagagatcaaccagattttcaccaagatgaataaggtgtctgaag
tgtacccggaaaaggaggaactgaaagagcttccacctgtcacttcggaa
cagtga.

In some embodiments, the engineered immune cells comprise a heterologous nucleic acid sequence that is at least 80%, 85%, 90%, 95%, 96%, 97%, 98%, or 99% identical to SEQ ID NO: 7, SEQ ID NO: 8 or SEQ ID NO: 9. Additionally or alternatively, in some embodiments, the expression levels and/or activity of GLUT5 in the engineered immune cell is at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 times higher compared to that observed in a native immune cell, wherein the engineered immune cell is of the same lineage as the native immune cell.

In some embodiments, the engineered immune cell further comprises a first regulatory sequence operatively linked to the nucleic acid encoding the GLUT5. In further embodiments, the first regulatory sequence directs the expression of the GLUT5. Additionally or alternatively, in some embodiments, the first regulatory sequence comprises, or consists essentially of, or yet further consists of a promoter, for example a constitutive promoter or a conditional promoter. In further embodiments, the conditional promoter is an immune cell specific promoter.

In one aspect, the engineered immune cells provided herein overexpress GLUT5 and/or comprise a heterologous nucleic acid encoding the GLUT5 gene. In certain embodiments, the engineered immune cells of the present disclosure target and kill a cancer cell expressing a target antigen more efficiently at a tissue site. The engineered immune cells disclosed herein can be generated by in vitro transduction of immune cells with a nucleic acid as disclosed herein.

### Receptors

Typical therapeutic anti-cancer monoclonal antibody (mAb), like those that bind to CD19, recognize cell surface proteins, which constitute only a tiny fraction of the cellular protein content. Most mutated or oncogenic tumor associated proteins are typically nuclear or cytoplasmic. In certain instances, these intracellular proteins can be degraded in the proteasome, processed and presented on the cell surface by MHC class I molecules as T cell epitopes that are recognized by T cell receptors (TCRs). The development of mAb that mimic TCR function, “TCR mimic (TCRm)” or “TCR-like”; (i.e., that recognize peptide antigens of key intracellular proteins in the context of MHC on the cell surface) greatly extends the potential repertoire of tumor targets addressable by potent mAb. TCRm Fab, or scFv, and mouse IgG specific for the melanoma Ags, NY-ESO-1, hTERT, MART 1, gp100, and PR1, among others, have been developed. The antigen binding portions of such antibodies can be incorporated into the CARs provided herein. HLA-A2 is the most common HLA haplotype in the USA and EU (about 40% of the population) (Marsh, S., Parham, P., Barber, L., The HLA FactsBook. 1 ed. The HLA FactsBook. Vol. 1. 2000: Academic Press. 416). Therefore, potent TCRm mAb and native TCRs against tumor antigens presented in the context of HLA-A2 are useful in the treatment of a large populations.

Accordingly, in some embodiments, a receptor as disclosed herein binds to a target antigen. In further embodiments, the target antigen is a tumor antigen presented in the context of an MHC molecule. In some embodiments, the MHC protein is a MHC class I protein. In some embodiments, the MHC Class I protein is an HLA-A, HLA-B, or HLA-C molecules. In some embodiments, target antigen is a tumor antigen presented in the context of an HLA-A2 molecule.

In some embodiments, the engineered immune cells provided herein express at least one chimeric antigen receptor (CAR). CARs are engineered receptors, which graft or confer a specificity of interest onto an immune effector cell. For example, CARs can be used to graft the specificity of a monoclonal antibody onto an immune cell, such as a T cell. In some embodiments, transfer of the coding sequence of the CAR is facilitated by nucleic acid vector, such as a retroviral vector.

There are currently three generations of CARs. In some embodiments, the engineered immune cells provided herein express a “first generation” CAR. “First generation” CARs are typically composed of an extracellular antigen binding domain (e.g., a single-chain variable fragment (scFv)) fused to a transmembrane domain fused to cytoplasmic/intracellular domain of the T cell receptor (TCR) chain. “First generation” CARs typically have the intracellular domain from the CD3ζ chain, which is the primary transmitter of signals from endogenous TCRs. “First generation” CARs can provide de novo antigen recognition and cause activation of both CD4⁺ and CD8⁺ T cells through their CD3ζ chain signaling domain in a single fusion molecule, independent of HLA-mediated antigen presentation.

In some embodiments, the engineered immune cells provided herein express a “second generation” CAR. “Second generation” CARs add intracellular domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (e.g., CD3ζ). Preclinical studies have indicated that “Second Generation” CARs can improve the antitumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL).

In some embodiments, the engineered immune cells provided herein express a “third generation” CAR. “Third generation” CARs comprise those that provide multiple co-stimulation (e.g., CD28 and 4-1BB) and activation (e.g., CD3ζ).

In accordance with the presently disclosed subject matter, the CARs of the engineered immune cells provided herein comprise an extracellular antigen-binding domain, a transmembrane domain and an intracellular domain. Further, the activity of the engineered immune cells can be adjusted by selection of co-stimulatory molecules included in the chimeric antigen receptor.

Extracellular Antigen-Binding Domain of a CAR. In certain embodiments, the extracellular antigen-binding domain of a CAR specifically binds a target antigen. In certain embodiments, the extracellular antigen-binding domain is derived from a monoclonal antibody (mAb) that binds to a target antigen. In some embodiments, the extracellular antigen-binding domain comprises, or consists essentially of, or yet further consists of an scFv. In some embodiments, the extracellular antigen-binding domain comprises, or consists essentially of, or yet further consists of a Fab, which is optionally crosslinked. In some embodiments, the extracellular binding domain comprises, or consists essentially of, or yet further consists of a F(ab)₂. In some embodiments, any of the foregoing molecules are included in a fusion protein with a heterologous sequence to form the extracellular antigen-binding domain. In certain embodiments, the extracellular antigen-binding domain comprises, or consists essentially of, or yet further consists of a human scFv that binds specifically to a target antigen. In certain embodiments, the scFv is identified by screening scFv phage library with a target antigen-Fc fusion protein.

In certain embodiments, the extracellular antigen-binding domain of a presently disclosed CAR has a high binding specificity and high binding affinity to a target antigen. For example, in some embodiments, the extracellular antigen-binding domain of the CAR (embodied, for example, in a human scFv or an analog thereof) binds to a particular target antigen with a dissociation constant (K_{d}) of about 1×10⁻⁵ M or less. In certain embodiments, the K_{d} is about 5×10⁻⁶ M or less, about 1×10⁻⁶ M or less, about 5×10⁻⁷ M or less, about 1×10⁻⁷ M or less, about 5×10⁻⁸ M or less, about 1×10⁻⁸ M or less, about 5×10⁻⁹ or less, about 4×10⁻⁹ or less, about 3×10⁻⁹ or less, about 2×10⁻⁹ or less, or about 1×10⁻⁹ M or less. In certain non-limiting embodiments, the K_{d} is from about 3×10⁻⁹ M or less. In certain non-limiting embodiments, the K_{d} is from about 3×10⁻⁹ to about 2×10⁻⁷.

Binding of the extracellular antigen-binding domain (embodiment, for example, in an scFv or an analog thereof) of a presently disclosed target-antigen-specific CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the target-antigen-specific CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In certain embodiments, the scFv of a presently disclosed target-antigen-specific CAR is labeled with GFP.

In some embodiments, the extracellular antigen-binding domain of the expressed CAR binds to a target antigen that is expressed by a tumor cell. In some embodiments, the extracellular antigen-binding domain of the expressed CAR binds to a target antigen that is expressed on the surface of a tumor cell. In some embodiments, the extracellular antigen-binding domain of the expressed CAR binds to a target antigen that is expressed on the surface of a tumor cell in combination with an MHC protein. In some embodiments, the MHC protein is a MHC class I protein. In some embodiments, the MHC Class I protein is an HLA-A, HLA-B, or HLA-C molecules. In some embodiments, the extracellular antigen-binding domain of the expressed CAR binds to a target antigen that is expressed on the surface of a tumor cell not in combination with an MHC protein.

In some embodiments, the extracellular antigen-binding domain of the expressed CAR binds to a target antigen. In some embodiments, the extracellular antigen-binding domain of the expressed CAR binds to a target antigen presented in the context of an MHC molecule. In some embodiments, the extracellular antigen-binding domain of the expressed CAR binds to a target antigen presented in the context of an HLA-A2 molecule.

In certain embodiments, the extracellular antigen-binding domain (e.g., human scFv) comprises a heavy chain variable (V_{H}) region and a light chain variable (V_{L}) region, optionally linked with a linker sequence, for example a linker peptide (e.g., SEQ ID NO: 3), between the heavy chain variable (V_{H}) region and the light chain variable (V_{L}) region. In certain embodiments, the extracellular antigen-binding domain is a human scFv-Fc fusion protein or full length human IgG with V_{H} and V_{L} regions.

In certain non-limiting embodiments, an extracellular antigen-binding domain of the presently disclosed CAR can comprise a linker connecting the heavy chain variable (V_{H}) region and light chain variable (V_{L}) region of the extracellular antigen-binding domain. As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises amino acids having the sequence set forth in SEQ ID NO: 3. In certain embodiments, the nucleotide sequence encoding the amino acid sequence of SEQ ID NO: 3 is set forth in SEQ ID NO: 4.

Additionally or alternatively, in some embodiments, the extracellular antigen-binding domain can comprise a leader or a signal peptide sequence that directs the nascent protein into the endoplasmic reticulum. The signal peptide or leader can be essential if the CAR is to be glycosylated and anchored in the cell membrane. The signal sequence or leader sequence can be a peptide sequence (about 5, about 10, about 15, about 20, about 25, or about 30 amino acids long) present at the N-terminus of the newly synthesized proteins that direct their entry to the secretory pathway.

In certain embodiments, the signal peptide is covalently joined to the N-terminus of the extracellular antigen-binding domain. In certain embodiments, the signal peptide comprises a human CD8 signal polypeptide comprising amino acids having the sequence set forth in SEQ ID NO: 10 as provided below: MALPVTALLLPLALLLHAARP (SEQ ID NO: 10).

The nucleotide sequence encoding the amino acid sequence of SEQ ID NO: 10 is set forth in SEQ ID NO: 11, which is provided below:

(SEQ ID NO: 11)
ATGGCCCTGCCAGTAACGGCTCTGCTGCTGCCACTTGCTCTGCTCCTCCA
TGCAGCCAGGCCT.

In certain embodiments, the signal peptide comprises a human CD8 signal polypeptide comprising amino acids having the sequence set forth in SEQ ID NO: 12 as provided below: MALPVTALLLPLALLLHA (SEQ ID NO: 12).

The nucleotide sequence encoding the amino acid sequence of SEQ ID NO: 12 is set forth in SEQ ID NO: 13, which is provided below:

(SEQ ID NO: 13)
ATGGCTCTCCCAGTGACTGCCCTACTGCTTCCCCTAGCGCTTCTCCTGCA
TGCA.

In certain embodiments, the signal peptide comprises a mouse CD8 signal polypeptide comprising amino acids having the sequence set forth in SEQ ID NO: 14 as provided below: MASPLTRFLSLNLLLLGESII (SEQ ID NO: 14).

The nucleotide sequence encoding the amino acid sequence of SEQ ID NO: 14 is set forth in SEQ ID NO: 15, which is provided below:

(SEQ ID NO: 15)
ATGGCCAGCCCCCTGACCAGGTTCCTGAGCCTGAACCTGCTGCTGCTGGG
CGAGAGCATCATC.

In certain embodiments, the signal peptide comprises a mouse CD8 signal polypeptide comprising amino acids having the sequence set forth in SEQ ID NO: 16 as provided below: MASPLTRFLSLNLLLLGE (SEQ ID NO: 16).

The nucleotide sequence encoding the amino acid sequence of SEQ ID NO: 16 is set forth in SEQ ID NO: 17, which is provided below:

(SEQ ID NO: 17)
ATGGCCAGCCCCCTGACCAGGTTCCTGAGCCTGAACCTGCTGCTGCTGGG
CGAG.

Transmembrane Domain of a CAR. In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide (e.g., a transmembrane peptide not based on a protein associated with the immune response), or a combination thereof.

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous to the sequence having a UniProtKB Reference No: P10747 or NCBI Reference No: NP006130 (SEQ ID NO: 18), or fragments thereof, and/or may optionally comprise up to one or up to two or up to three conservative amino acid substitutions. In certain embodiments, the CD28 polypeptide can have an amino acid sequence that is a consecutive portion of SEQ ID NO: 18 which is at least 20, or at least 30, or at least 40, or at least 50, and up to 220 amino acids in length. Additionally or alternatively, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO: 18. In certain embodiments, the CAR of the present disclosure comprises a transmembrane domain comprising a CD28 polypeptide, and optionally an intracellular domain comprising a co-stimulatory signaling region that comprises a CD28 polypeptide. In certain embodiments, the CD28 polypeptide comprised in the transmembrane domain and the intracellular domain has an amino acid sequence of amino acids 114 to 220 of SEQ ID NO: 18. In certain embodiments, the CD28 polypeptide comprised in the transmembrane domain has an amino acid sequence of amino acids 153 to 179 of SEQ ID NO: 18.

SEQ ID NO: 18 is provided below:

(SEQ ID NO: 18)
MLRLLLALNLFPSIQVTGNKILVKQSPMLVAYDNALSCKYSYNLFSREFR
ASLHKGLDSAVEVCWYGNYSQQLQVYSKTGFNCDGKLGNESVTFYLQNLY
QTDIYFCKIEVMYPPPYLDNEKSNGTIIHVKGKHLCPSPLFPGPSKPFWV
LVWGGVLACYSLLVTVAFIIFWVRSKRSRLLHSDYMNMTPRRPGPTRKHY
QPYAPPRDFAAYRS

In accordance with the presently disclosed subject matter, a “CD28 nucleic acid molecule” refers to a polynucleotide encoding a CD28 polypeptide. In certain embodiments, the CD28 nucleic acid molecule encoding the CD28 polypeptide comprised in the transmembrane domain (and optionally the intracellular domain (e.g., the co-stimulatory signaling region)) of the presently disclosed CAR (e.g., amino acids 114 to 220 of SEQ ID NO: 18 or amino acids 153 to 179 of SEQ ID NO: 18) comprises at least a portion of the sequence set forth in SEQ ID NO: 19 as provided below.

(SEQ ID NO: 19)
attgaagttatgtatcctcctccttacctagacaatgagaagagcaatgg
aaccattatccatgtgaaagggaaacacctttgtccaagtcccctatttc
ccggaccttctaagcccttttgggtgctggtggtggttggtggagtcctg
gcttgctatagcttgctagtaacagtggcctttattattttctgggtgag
gagtaagaggagcaggctcctgcacagtgactacatgaacatgactcccc
gccgccccgggcccacccgcaagcattaccagccctatgccccaccacgc
gacttcgcagcctatcgctcc

In certain embodiments, the transmembrane domain comprises a CD8 polypeptide. The CD8 polypeptide can have an amino acid sequence that is at least about 85%, about 90%, about 95%, about 96%, about 97%, about 98%, about 99% or about 100%) homologous to SEQ ID NO: 20 (homology herein may be determined using standard software such as BLAST or FASTA) as provided below, or fragments thereof, and/or may optionally comprise up to one or up to two or up to three conservative amino acid substitutions. In certain embodiments, the CD8 polypeptide can have an amino acid sequence that is a consecutive portion of SEQ ID NO: 20 which is at least 20, or at least 30, or at least 40, or at least 50, and up to 235 amino acids in length. Additionally or alternatively, in various embodiments, the CD8 polypeptide has an amino acid sequence of amino acids 1 to 235, 1 to 50, 50 to 100, 100 to 150, 150 to 200, or 200 to 235 of SEQ ID NO: 20.

(SEQ ID NO: 20)
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPWKSGDKPSLSARYV

In certain embodiments, the transmembrane domain comprises a CD8 polypeptide comprising amino acids having the sequence set forth in SEQ ID NO: 21 as provided below:

(SEQ ID NO: 21)
PTTTPAPRPPTPAPTIASQPLSLRPEACRPAAGGAVHTRGLDFACDIYIW
APLAGTCGVLLLSLVITLYCN

In accordance with the presently disclosed subject matter, a “CD8 nucleic acid molecule” refers to a polynucleotide encoding a CD8 polypeptide. In certain embodiments, the CD8 nucleic acid molecule encoding the CD8 polypeptide comprised in the transmembrane domain of the presently disclosed CAR (SEQ ID NO: 21) comprises nucleic acids having the sequence set forth in SEQ ID NO: 22 as provided below.

(SEQ ID NO: 22)
CCCACCACGACGCCAGCGCCGCGACCACCAACCCCGGCGCCCACGATCGC
GTCGCAGCCCCTGTCCCTGCGCCCAGAGGCGTGCCGGCCAGCGGCGGGGG
GCGCAGTGCACACGAGGGGGCTGGACTTCGCCTGTGATATCTACATCTGG
GCGCCCCTGGCCGGGACTTGTGGGGTCCTTCTCCTGTCACTGGTTATCAC
CCTTTACTGCAAC

In certain non-limiting embodiments, a CAR can also comprise a spacer region that links the extracellular antigen-binding domain to the transmembrane domain. The spacer region can be flexible enough to allow the antigen-binding domain to orient in different directions to facilitate antigen recognition while preserving the activating activity of the CAR. In certain non-limiting embodiments, the spacer region can be the hinge region from IgG1, the CH₂CH₃ region of immunoglobulin and portions of CD3, a portion of a CD28 polypeptide (e.g., SEQ ID NO: 18), a portion of a CD8 polypeptide (e.g., SEQ ID NO: 20), a variation of any of the foregoing which is at least about 80%, at least about 85%, at least about 90%, or at least about 95% homologous thereto, or a synthetic spacer sequence. In certain non-limiting embodiments, the spacer region may have a length between about 1-50 (e.g., 5-25, 10-30, or 30-50) amino acids.

Intracellular Domain of a CAR. In certain non-limiting embodiments, an intracellular domain of the CAR can comprise a CD3ζ polypeptide, which can activate or stimulate a cell (e.g., a cell of the lymphoid lineage, e.g., a T cell). CD3ζ comprises 3 ITAMs, and transmits an activation signal to the cell (e.g., a cell of the lymphoid lineage, e.g., a T cell) after antigen is bound. The CD3ζ polypeptide can have an amino acid sequence that is at least about 85%, about 90%, about 95%, about 96%, about 97%, about 98%, about 99% or about 100% homologous to the sequence having a NCBI Reference No: NP_932170 (SEQ ID NO: 23), or fragments thereof, and/or may optionally comprise up to one or up to two or up to three conservative amino acid substitutions.

In certain embodiments, the CD3ζ polypeptide can have an amino acid sequence that is a consecutive portion of SEQ ID NO: 24 which is at least 20, or at least 30, or at least 40, or at least 50, and up to 164 amino acids in length. Additionally or alternatively, in various embodiments, the CD3ζ polypeptide has an amino acid sequence of amino acids 1 to 164, 1 to 50, 50 to 100, 100 to 150, or 150 to 164 of SEQ ID NO: 24. In certain embodiments, the CD3ζ polypeptide has an amino acid sequence of amino acids 52 to 164 of SEQ ID NO: 24.

SEQ ID NO: 24 is provided below:

(SEQ ID NO: 24)
MKWKALFTAAILQAQLPITEAQSFGLLDPKLCYLLDGILFIYGVI
LTALFLRVKFSRSADAPAYQQGQNQLYNELNLGRREEYDVLDKRR
GRDPEMGGKPQRRKNPQEGLYNELQKDKMAEAYSEIGMKGERRRG
KGHDGLYQGLSTATKDTYDALHMQALPPR

In certain embodiments, the CD3ζ polypeptide has the amino acid sequence set forth in SEQ ID NO: 25, which is provided below:

(SEQ ID NO: 25)
RVKFSRSAEPPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR

In certain embodiments, the CD3ζ polypeptide has the amino acid sequence set forth in SEQ ID NO: 26, which is provided below:

(SEQ ID NO: 26)
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR

In accordance with the presently disclosed subject matter, a “CD3ζ nucleic acid molecule” refers to a polynucleotide encoding a CD3ζ polypeptide. In certain embodiments, the CD3ζ nucleic acid molecule encoding the CD3ζ polypeptide (SEQ ID NO: 25) comprised in the intracellular domain of the presently disclosed CAR comprises a nucleotide sequence as set forth in SEQ ID NO: 27 as provided below.

(SEQ ID NO: 27)
AGAGTGAAGTTCAGCAGGAGCGCAGAGCCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGAGAAGGAAGAACCCTCAGGAAGGCCTGTACAAT
GAACTGCAGAAAGATAAGATGGCGGAGGCCTACAGTGAGATTGGG
ATGAAAGGCGAGCGCCGGAGGGGCAAGGGGCACGATGGCCTTTAC
CAGGGTCTCAGTACAGCCACCAAGGACACCTACGACGCCCTTCAC
ATGCAGGCCCTGCCCCCTCGCG

In certain embodiments, the CD3ζ nucleic acid molecule encoding the CD3ζ polypeptide (SEQ ID NO: 26) comprised in the intracellular domain of the presently disclosed CAR comprises a nucleotide sequence as set forth in SEQ ID NO: 28 as provided below.

(SEQ ID NO: 28)
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGAGAAGGAAGAACCCTCAGGAAGGCCTGTACAAT
GAACTGCAGAAAGATAAGATGGCGGAGGCCTACAGTGAGATTGGG
ATGAAAGGCGAGCGCCGGAGGGGCAAGGGGCACGATGGCCTTTAC
CAGGGTCTCAGTACAGCCACCAAGGACACCTACGACGCCCTTCAC
ATGCAGGCCCTGCCCCCTCGCTAA

In certain non-limiting embodiments, an intracellular domain of the CAR further comprises at least one signaling region. The at least one signaling region can include a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof.

In certain embodiments, the signaling region is a co-stimulatory signaling region.

In certain embodiments, the co-stimulatory signaling region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, 4-1BBL, CD48, TNFRSF14, and PD-L1. As one example, a 4-1BB ligand (i.e., 4-1BBL) may bind to 4-1BB (also known as “CD 137”) for providing an intracellular signal that in combination with a CAR signal induces an effector cell function of the CAR⁺ T cell. CARs comprising an intracellular domain that comprises a co-stimulatory signaling region comprising 4-1BB, ICOS or DAP-10 are disclosed in U.S. Pat. No. 7,446,190, which is herein incorporated by reference in its entirety. In certain embodiments, the intracellular domain of the CAR comprises a co-stimulatory signaling region that comprises a CD28 polypeptide. In certain embodiments, the intracellular domain of the CAR comprises a co-stimulatory signaling region that comprises two co-stimulatory molecules: CD28 and 4-1BB or CD28 and OX40.

4-1BB can act as a tumor necrosis factor (TNF) ligand and have stimulatory activity. The 4-1BB polypeptide can have an amino acid sequence that is at least about 85%, about 90%, about 95%, about 96%, about 97%, about 98%, about 99% or 100% homologous to the sequence having a UniProtKB Reference No: P41273 or NCBI Reference No: NP_001552 (SEQ ID NO: 29) or fragments thereof, and/or may optionally comprise up to one or up to two or up to three conservative amino acid substitutions.

SEQ ID NO: 29 is provided below:

(SEQ ID NO: 29)
MGNSCYNIVATLLLVLNFERTRSLQDPCSNCPAGTFCDNNRNQIC
SPCPPNSFSSAGGQRTCDICRQCKGVFRTRKECSSTSNAECDCTP
GFHCLGAGCSMCEQDCKQGQELTKKGCKDCCFGTFNDQKRGICRP
WTNCSLDGKSVLGTKERDWCGPSPADLSPGASSVTPPAPAREPGH
SPQIISFFLALTSTALLFLLFFLTLRFSWKRGRKKLLYIFKQPFM
RPVQTTQEEDGCSCRFPEEEEGGCEL

In certain embodiments, the 4-1BB co-stimulatory domain has the amino acid sequence set forth in SEQ ID NO: 30, which is provided below:

(SEQ ID NO: 30)
KRGRKKLLYIFKQPFMRPVQTTQEEDGCSCRFPEEEEGGCEL

In accordance with the presently disclosed subject matter, a “4-1BB nucleic acid molecule” refers to a polynucleotide encoding a 4-1BB polypeptide. In certain embodiments, the 4-1BB nucleic acid molecule encoding the 4-1BB polypeptide (SEQ ID NO: 30) comprised in the intracellular domain of the presently disclosed CAR comprises a nucleotide sequence as set forth in SEQ ID NO: 31 as provided below.

(SEQ ID NO: 31)
AAACGGGGCAGAAAGAAGCTCCTGTATATATTCAAACAACCATTT
ATGAGACCAGTACAAACTACTCAAGAGGAAGATGGCTGTAGCTGC
CGATTTCCAGAAGAAGAAGAAGGAGGATGTGAACTG

An OX40 polypeptide can have an amino acid sequence that is at least about 85%, about 90%, about 95%, about 96%, about 97%, about 98%, about 99% or 100% homologous to the sequence having a UniProtKB Reference No: P43489 or NCBI Reference No: NP_003318 (SEQ ID NO: 32), or fragments thereof, and/or may optionally comprise up to one or up to two or up to three conservative amino acid substitutions.

SEQ ID NO: 32 is provided below:

(SEQ ID NO: 32)
MCVGARRLGRGPCAALLLLGLGLSTVTGLHCVGDTYPSNDRCCHE
CRPGNGMVSRCSRSQNTVCRPCGPGFYNDWSSKPCKPCTWCNLRS
GSERKQLCTATQDTVCRCRAGTQPLDSYKPGVDCAPCPPGHFSPG
DNQACKPWTNCTLAGKHTLQPASNSSDAICEDRDPPATQPQETQG
PPARPITVQPTEAWPRTSQGPSTRPVEVPGGRAVAAILGLGLVLG
LLGPLAILLALYLLRRDQRLPPDAHKPPGGGSFRTPIQEEQADAH
STLAKI

In accordance with the presently disclosed subject matter, an “OX40 nucleic acid molecule” refers to a polynucleotide encoding an OX40 polypeptide.

An ICOS polypeptide can have an amino acid sequence that is at least about 85%, about 90%, about 95%, about 96%, about 97%, about 98%, about 99% or 100% homologous to the sequence having a NCBI Reference No: NP_036224 (SEQ ID NO: 33) or fragments thereof, and/or may optionally comprise up to one or up to two or up to three conservative amino acid substitutions.

SEQ ID NO: 33 is provided below:

(SEQ ID NO: 33)
MKSGLWYFFLFCLRIKVLTGEINGSANYEMFIFHNGGVQILCKYP
DIVQQFKMQLLKGGQILCDLTKTKGSGNTVSIKSLKFCHSQLSNN
SVSFFLYNLDHSHANYYFCNLSIFDPPPFKVTLTGGYLHIYESQL
CCQLKFWLPIGCAAFVWCILGCILICWLTKKKYSSSVHDPNGEYM
FMRATAKKSRLTDVTL

In accordance with the presently disclosed subject matter, an “ICOS nucleic acid molecule” refers to a polynucleotide encoding an ICOS polypeptide.

CTLA-4 is an inhibitory receptor expressed by activated T cells, which when engaged by its corresponding ligands (CD80 and CD86; B7-1 and B7-2, respectively), mediates activated T cell inhibition or anergy. In both preclinical and clinical studies, CTLA-4 blockade by systemic antibody infusion, enhanced the endogenous anti-tumor response albeit, in the clinical setting, with significant unforeseen toxicities.

CTLA-4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM (SEQ ID NO: 34) motif able to bind PI3K, PP2A and SHP-2 and one proline-rich motif able to bind SH3 containing proteins. One role of CTLA-4 in inhibiting T cell responses seem to be directly via SHP-2 and PP2A dephosphorylation of TCR-proximal signaling proteins such as CD3 and LAT. CTLA-4 can also affect signaling indirectly via competing with CD28 for CD80/86 binding. CTLA-4 has also been shown to bind and/or interact with PI3K, CD80, AP2M1, and PPP2R5A.

In accordance with the presently disclosed subject matter, a CTLA-4 polypeptide can have an amino acid sequence that is at least about 85%, about 90%, about 95%, about 96%, about 97%, about 98%, about 99% or about 100% homologous to UniProtKB/Swiss-Prot Ref. No.: P16410.3 (SEQ ID NO: 35) (homology herein may be determined using standard software such as BLAST or FASTA) or fragments thereof, and/or may optionally comprise up to one or up to two or up to three conservative amino acid substitutions.

SEQ ID NO: 35 is provided below:

(SEQ ID NO: 35)
MACLGFQRHKAQLNLATRTWPCTLLFFLLFIPVFCKAMHVAQPAW
LASSRGIASFVCEYASPGKATEVRVTVLRQADSQVTEVCAATYMM
GNELTFLDDSICTGTSSGNQLTIQGLRAMDTGLYICKVELMYPPP
YYLGIGNGTQIYVIDPEPCPDSDFLLWILAAVSSGLFFYSFLLTA
VSLSKMLKKRSPLTTGVYVKMPPTEPECEKQFQPYFIPIN

In accordance with the presently disclosed subject matter, a “CTLA-4 nucleic acid molecule” refers to a polynucleotide encoding a CTLA-4 polypeptide.

PD-1 is a negative immune regulator of activated T cells upon engagement with its corresponding ligands PD-L1 and PD-L2 expressed on endogenous macrophages and dendritic cells. PD-1 is a type I membrane protein of 268 amino acids. PD-1 has two ligands, PD-L1 and PD-L2, which are members of the B7 family. The protein's structure comprises an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, that PD-1 negatively regulates TCR signals. SHP-I and SHP-2 phosphatases bind to the cytoplasmic tail of PD-1 upon ligand binding. Upregulation of PD-L1 is one mechanism tumor cells may evade the host immune system. In pre-clinical and clinical trials, PD-1 blockade by antagonistic antibodies induced anti-tumor responses mediated through the host endogenous immune system. In accordance with the presently disclosed subject matter, a PD-1 polypeptide can have an amino acid sequence that is at least about 85%, about 90%, about 95%, about 96%, about 97%, about 98%, about 99% or about 100% homologous to NCBI Reference No: NP_005009.2 (SEQ ID NO: 36) or fragments thereof, and/or may optionally comprise up to one or up to two or up to three conservative amino acid substitutions.

SEQ ID NO: 36 is provided below:

(SEQ ID NO: 36)
MQIPQAPWPVVWAVLQLGWRPGWFLDSPDRPWNPPTFSPALLWTE
GDNATFTCSFSNTSESFVLNWYRMSPSNQTDKLAAFPEDRSQPGQ
DCRFRVTQLPNGRDFHMSVVRARRNDSGTYLCGAISLAPKAQIKE
SLRAELRVTERRAEVPTAHPSPSPRPAGQFQTLVVGWGGLLGSLV
LLVWVLAVICSRAARGTIGARRTGQPLKEDPSAVPVFSVDYGELD
FQWREKTPEPPVPCVPEQTEYATIVFPSGMGTSSPARRGSADGPR
SAQPLRPEDGHCSWPL

In accordance with the presently disclosed subject matter, a “PD-1 nucleic acid molecule” refers to a polynucleotide encoding a PD-1 polypeptide.

Lymphocyte-activation protein 3 (LAG-3) is a negative immune regulator of immune cells. LAG-3 belongs to the immunoglobulin (Ig) superfamily and contains 4 extracellular Ig-like domains. The LAG3 gene contains 8 exons. The sequence data, exon/intron organization, and chromosomal localization all indicate a close relationship of LAG3 to CD4. LAG3 has also been designated CD223 (cluster of differentiation 223).

In accordance with the presently disclosed subject matter, a LAG-3 polypeptide can have an amino acid sequence that is at least about 85%, about 90%, about 95%, about 96%, about 97%, about 98%, about 99% or about 100% homologous to UniProtKB/Swiss-Prot Ref. No.: P18627.5 (SEQ ID NO: 37) or fragments thereof, and/or may optionally comprise up to one or up to two or up to three conservative amino acid substitutions.

SEQ ID NO: 37 is provided below:

(SEQ ID NO: 37)
MWEAQFLGLLFLQPLWVAPVKPLQPGAEVPWWAQEGAPAQLPCSP
TIPLQDLSLLRRAGVTWQHQPDSGPPAAAPGHPLAPGPHPAAPSS
WGPRPRRYTVLSVGPGGLRSGRLPLQPRVQLDERGRQRGDFSLWL
RPARRADAGEYRAAVHLRDRALSCRLRLRLGQASMTASPPGSLRA
SDWVILNCSFSRPDRPASVHWFRNRGQGRVPVRESPHHHLAESFL
FLPQVSPMDSGPWGCILTYRDGFNVSIMYNLTVLGLEPPTPLTVY
AGAGSRVGLPCRLPAGVGTRSFLTAKWTPPGGGPDLLVTGDNGDF
TLRLEDVSQAQAGTYTCHIHLQEQQLNATVTLAIITVTPKSFGSP
GSLGKLLCEVTPVSGQERFVWSSLDTPSQRSFSGPWLEAQEAQLL
SQPWQCQLYQGERLLGAAVYFTELSSPGAQRSGRAPGALPAGHLL
LFLILGVLSLLLLVTGAFGFHLWRRQWRPRRFSALEQGIHPPQAQ
SKIEELEQEPEPEPEPEPEPEPEPEPEQL

In accordance with the presently disclosed subject matter, a “LAG-3 nucleic acid molecule” refers to a polynucleotide encoding a LAG-3 polypeptide.

Natural Killer Cell Receptor 2B4 (2B4) mediates non-MHC restricted cell killing on NK cells and subsets of T cells. To date, the function of 2B4 is still under investigation, with the 2B4-S isoform believed to be an activating receptor, and the 2B4-L isoform believed to be a negative immune regulator of immune cells. 2B4 becomes engaged upon binding its high-affinity ligand, CD48. 2B4 contains a tyrosine-based switch motif, a molecular switch that allows the protein to associate with various phosphatases. 2B4 has also been designated CD244 (cluster of differentiation 244).

In accordance with the presently disclosed subject matter, a 2B4 polypeptide can have an amino acid sequence that is at least about 85%, about 90%, about 95%, about 96%, about 97%, about 98%, about 99% or about 100% homologous to UniProtKB/Swiss-Prot Ref. No.: Q9BZW8.2 (SEQ ID NO: 38) or fragments thereof, and/or may optionally comprise up to one or up to two or up to three conservative amino acid substitutions.

SEQ ID NO: 38 is provided below:

(SEQ ID NO: 38)
MLGQWTLILLLLLKVYQGKGCQGSADHWSISGVPLQLQPNSIQTK
VDSIAWKKLLPSQNGFHHILKWENGSLPSNTSNDRFSFIVKNLSL
LIKAAQQQDSGLYCLEVTSISGKVQTATFQVFVFESLLPDKVEKP
RLQGQGKILDRGRCQVALSCLVSRDGNVSYAWYRGSKLIQTAGNL
TYLDEEVDINGTHTYTCNVSNPVSWESHTLNLTQDCQNAHQEFRF
WPFLVIIVILSALFLGTLACFCVWRRKRKEKQSETSPKEFLTIYE
DVKDLKTRRNHEQEQTFPGGGSTIYSMIQSQSSAPTSQEPAYTLY
SLIQPSRKSGSRKRNHSPSFNSTIYEVIGKSQPKAQNPARLSRKE
LENFDVYS

In accordance with the presently disclosed subject matter, a “2B4 nucleic acid molecule” refers to a polynucleotide encoding a 2B4 polypeptide.

B- and T-lymphocyte attenuator (BTLA) expression is induced during activation of T cells, and BTLA remains expressed on Th1 cells but not Th2 cells. Like PD1 and CTLA4, BTLA interacts with a B7 homolog, B7H4. However, unlike PD-1 and CTLA-4, BTLA displays T-Cell inhibition via interaction with tumor necrosis family receptors (TNF-R), not just the B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses. BTLA activation has been shown to inhibit the function of human CD8⁺ cancer-specific T cells. BTLA has also been designated as CD272 (cluster of differentiation 272).

In accordance with the presently disclosed subject matter, a BTLA polypeptide can have an amino acid sequence that is at least about 85%, about 90%, about 95%, about 96%, about 97%, about 98%, about 99% or about 100% homologous to UniProtKB/Swiss-Prot Ref. No.: Q7Z6A9.3 (SEQ ID NO: 39) or fragments thereof, and/or may optionally comprise up to one or up to two or up to three conservative amino acid substitutions.

SEQ ID NO: 39 is provided below:

(SEQ ID NO: 39)
MKTLPAMLGTGKLFWVFFLIPYLDIWNIHGKESCDVQLYIKRQSE
HSILAGDPFELECPVKYCANRPHVTWCKLNGTTCVKLEDRQTSWK
EEKNISFFILHFEPVLPNDNGSYRCSANFQSNLIESHSTTLYVTD
VKSASERPSKDEMASRPWLLYRLLPLGGLPLLITTCFCLFCCLRR
HQGKQNELSDTAGREINLVDAHLKSEQTEASTRQNSQVLLSETGI
YDNDPDLCFRMQEGSEVYSNPCLEENKPGIVYASLNHSVIGPNSR
LARNVKEAPTEYASICVRS

In accordance with the presently disclosed subject matter, a “BTLA nucleic acid molecule” refers to a polynucleotide encoding a BTLA polypeptide.

Additionally or alternatively, in certain embodiments, the heterologous GLUT5 nucleic acid and a first reporter or selection marker (e.g., GFP, LNGFR) are expressed as a single polypeptide linked by a self-cleaving linker, such as a P2A linker. In certain embodiments, the heterologous GLUT5 gene and a reporter or selection marker (e.g., GFP, LNGFR) are expressed as two separate polypeptides.

Additionally or alternatively, in certain embodiments, the receptor of the present technology and a second reporter or selection marker (e.g., GFP, LNGFR) are expressed as a single polypeptide linked by a self-cleaving linker, such as a P2A linker. In certain embodiments, the receptor and a second reporter or selection marker (e.g., GFP, LNGFR) are expressed as two separate polypeptides.

Additionally or alternatively, in some embodiments, the heterologous nucleic acid encoding the GLUT5 gene and/or any receptor disclosed herein is operably linked to an inducible promoter. In some embodiments, the heterologous nucleic acid encoding the GLUT5 gene and/or any receptor disclosed herein is operably linked to a constitutive promoter.

In some embodiments, the inducible promoter is a synthetic Notch promoter that is activatable in a CAR T cell, where the intracellular domain of the CAR contains a transcriptional regulator that is released from the membrane when engagement of the CAR with the target antigen/polypeptide induces intramembrane proteolysis (see, e.g., Morsut et al., Cell 164(4): 780-791 (2016). Accordingly, further transcription of the target-antigen-specific CAR is induced upon binding of the engineered immune cell with the antigen/polypeptide.

The presently disclosed subject matter also provides isolated nucleic acid molecules encoding the GLUT5 gene and CAR constructs described herein or a functional portion thereof. In certain embodiments, the CAR construct comprises (a) an antigen binding fragment (e.g., an anti-target-antigen scFv or a fragment) that specifically binds to a target antigen, (b) a transmembrane domain comprising a CD8 polypeptide or CD28 polypeptide, and (c) an intracellular domain comprising a CD3ζ polypeptide, and optionally one or more of a co-stimulatory signaling region disclosed herein, a P2A self-cleaving peptide, and/or a reporter or selection marker (e.g., GFP, LNGFR) provided herein. The at least one co-stimulatory signaling region can include a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof.

In certain embodiments, the isolated nucleic acid molecule encodes a GLUT5 gene and a receptor (such as a CAR that specifically binds a target antigen) comprising an antigen binding fragment (e.g., a scFv) that specifically binds to a target antigen/polypeptide, fused to a synthetic Notch transmembrane domain and an intracellular cleavable transcription factor. In certain embodiments, the present disclosure provides an isolated nucleic acid molecule encoding a GLUT5 gene and a receptor (such as a CAR that specifically binds a target antigen) that is inducible by release of the transcription factor of a synthetic Notch system.

In certain embodiments, the isolated nucleic acid molecule encodes a functional portion of a presently disclosed CAR constructs. As used herein, the term “functional portion” refers to any portion, part or fragment of a CAR, which portion, part or fragment retains the biological activity of the parent CAR. For example, functional portions encompass the portions, parts or fragments of a target-antigen-specific CAR that retains the ability to recognize a target senescent cell, to treat cancer or a senescence-associated pathology, to a similar, same, or even a higher extent as the parent CAR. In certain embodiments, an isolated nucleic acid molecule encoding a functional portion of a target-antigen-specific CAR can encode a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent CAR.

The presently disclosed subject matter provides engineered immune cells expressing a GLUT5 and a target-antigen-specific T-cell receptor (e.g., a CAR) or other ligand that comprises an extracellular antigen-binding domain, a transmembrane domain and an intracellular domain, where the extracellular antigen-binding domain specifically binds a target antigen/polypeptide. In certain embodiments immune cells can be transduced with a presently disclosed CAR constructs such that the cells express the CAR. The presently disclosed subject matter also provides methods of using such cells for the treatment of cancer or senescence-associated pathology.

The engineered immune cells of the presently disclosed subject matter can be cells of the lymphoid lineage or myeloid lineage. The lymphoid lineage, comprising B, T, and natural killer (NK) cells, provides for the production of antibodies, regulation of the cellular immune system, detection of foreign agents in the blood, detection of cells foreign to the host, and the like. Non-limiting examples of immune cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., T_{EM} cells and TEMRA cells, Regulatory T cells (also known as suppressor T cells), Natural killer T cells, Mucosal associated invariant T cells, and T6 T cells. Cytotoxic T cells (CTL or killer T cells) are a subset of T lymphocytes capable of inducing the death of infected somatic or tumor cells. In certain embodiments, the CAR-expressing T cells express Foxp3 to achieve and maintain a T regulatory phenotype.

Natural killer (NK) cells can be lymphocytes that are part of cell-mediated immunity and act during the innate immune response. NK cells do not require prior activation in order to perform their cytotoxic effect on target cells.

The engineered immune cells of the presently disclosed subject matter can express a GLUT5 and an extracellular antigen binding domain (e.g., an anti-target-antigen scFv, an anti-target-antigen Fab that is optionally crosslinked, an anti-target-antigen F(ab)₂ or a fragment) that specifically binds to a target antigen, for the treatment of cancer.

In some embodiments, the higher the expression level of either or both of GLUT 5 and the receptor in an engineered immune cell, the greater cytotoxicity and cytokine production the engineered immune cell exhibits. An engineered immune cell (e.g., T cell) having a high GLUT5 expression level can utilize fructose, induce antigen-specific cytokine production or secretion and/or exhibit improved cytotoxicity relative to a tissue or a cell having a low or no expression level of GLUT5, e.g., about 2,000 or less, about 1,000 or less, about 900 or less, about 800 or less, about 700 or less, about 600 or less, about 500 or less, about 400 or less, about 300 or less, about 200 or less, about 100 or less of sites/cell. Additionally or alternatively, the cytotoxicity and cytokine production of a presently disclosed engineered immune cell (e.g., T cell) are proportional to the expression level of target antigen in a target tissue or a target cell. Additionally or alternatively, the cytotoxicity and cytokine production of a presently disclosed engineered immune cell (e.g., T cell) are proportional to the expression level of GLUT5 in the immune cell. For example, the higher the expression level, the greater cytotoxicity and cytokine production the engineered immune cell exhibits.

In certain embodiments, the antigen recognizing receptor is a chimeric co-stimulatory receptor (CCR). CCR is described in Krause, et al., J. Exp. Med. 188(4):619-626(1998), and US20020018783, the contents of which are incorporated by reference in their entireties. CCRs mimic co-stimulatory signals, but unlike, CARs, do not provide a T-cell activation signal, e.g., CCRs lack a CD3ζ polypeptide. CCRs provide co-stimulation, e.g., a CD28-like signal, in the absence of the natural co-stimulatory ligand on the antigen-presenting cell. A combinatorial antigen recognition, i.e., use of a CCR in combination with a CAR, can augment T-cell reactivity against the dual-antigen expressing cells, thereby improving selective targeting. Kloss et al., describe a strategy that integrates combinatorial antigen recognition, split signaling, and, critically, balanced strength of T-cell activation and costimulation to generate T cells that eliminate target cells that express a combination of antigens while sparing cells that express each antigen individually (Kloss et al., Nature Biotechnology 31(1):71-75 (2013)). With this approach, T-cell activation requires CAR-mediated recognition of one antigen, whereas costimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen. In certain embodiments, the CCR comprises (a) an extracellular antigen-binding domain that binds to an antigen different than the first target antigen, (b) a transmembrane domain, and (c) a co-stimulatory signaling region that comprises at least one co-stimulatory molecule, including, but not limited to, CD28, 4-1BB, OX40, ICOS, PD-1, CTLA-4, LAG-3, 2B4, and BTLA. In certain embodiments, the co-stimulatory signaling region of the CCR comprises one co-stimulatory signaling molecule. In certain embodiments, the one co-stimulatory signaling molecule is CD28. In certain embodiments, the one co-stimulatory signaling molecule is 4-1BB. In certain embodiments, the co-stimulatory signaling region of the CCR comprises two co-stimulatory signaling molecules. In certain embodiments, the two co-stimulatory signaling molecules are CD28 and 4-1BB. A second target antigen is selected so that expression of both the first target antigen and the second target antigen is restricted to the targeted cells (e.g., cancerous cells). Similar to a CAR, the extracellular antigen-binding domain can be an scFv, a Fab, a F(ab)₂; or a fusion protein with a heterologous sequence to form the extracellular antigen-binding domain. In certain embodiments, the CCR comprises an scFv that binds to CD138, transmembrane domain comprising a CD28 polypeptide, and a co-stimulatory signaling region comprising two co-stimulatory signaling molecules that are CD28 and 4-1BB.

In certain embodiments, the antigen recognizing receptor is a truncated CAR. A “truncated CAR” is different from a CAR by lacking an intracellular signaling domain. For example, a truncated CAR comprises an extracellular antigen-binding domain and a transmembrane domain, and lacks an intracellular signaling domain. In accordance with the presently disclosed subject matter, the truncated CAR has a high binding affinity to the second antigen expressed on the targeted cells. The truncated CAR functions as an adhesion molecule that enhances the avidity of a presently disclosed CAR, especially, one that has a low binding affinity to a target antigen, thereby improving the efficacy of the presently disclosed CAR or engineered immune cell (e.g., T cell) comprising the same. In certain embodiments, the truncated CAR comprises an extracellular antigen-binding domain that binds to a target antigen, a transmembrane domain comprising a CD8 polypeptide. A presently disclosed T cell comprises or is transduced to express a presently disclosed CAR targeting a target antigen and a truncated CAR targeting a target antigen. In certain embodiments, the targeted cells are solid tumor cells.

### Polynucleotides, Polypeptides and Analogs

Also included in the presently disclosed subject matter are GLUT5 polynucleotides and their corresponding polypeptides or fragments that may be modified in ways that enhance their anti-tumor activity when expressed in an engineered immune cell. The presently disclosed subject matter provides methods for optimizing an amino acid sequence or a nucleic acid sequence by producing an alteration in the sequence. Such alterations can comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%), about 98%, about 99% or more identity or homology with all or part of a naturally-occurring amino, acid sequence of the presently disclosed subject matter. The length of sequence comparison is at least about 5, about 10, about 15, about 20, about 25, about 50, about 75, about 100 or more amino acid residues. Again, in an exemplary approach to determining the degree of identity, a BLAST program can be used, with a probability score between e⁻³ and e⁻¹⁰⁰ indicating a closely related sequence. Modifications comprise in vivo and in vitro chemical derivatization of polypeptides, e.g., acetylation, carboxylation, phosphorylation, or glycosylation; such modifications can occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethyl sulfate or by site-specific mutagenesis as described in Sambrook, Fritsch and Maniatis, Molecular Cloning: A Laboratory Manual (2nd ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta (β) or gamma (γ) amino acids.

In addition to full-length polypeptides, the presently disclosed subject matter also provides fragments of any one of the polypeptides or peptide domains of the presently disclosed subject matter. A fragment can be at least about 5, about 10, about 13, or about 15 amino acids. In some embodiments, a fragment is at least about 20 contiguous amino acids, at least about 30 contiguous amino acids, or at least about 50 contiguous amino acids. In some embodiments, a fragment is at least about 60 to about 80, about 100, about 200, about 300 or more contiguous amino acids. Fragments of the presently disclosed subject matter can be generated by methods known to those of ordinary skill in the art or can result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein. Such analogs are administered according to methods of the presently disclosed subject matter. Such analogs can exceed the physiological activity of the original polypeptide. Methods of analog design are well known in the art, and synthesis of analogs can be carried out according to such methods by modifying the chemical structures such that the resultant analogs increase the antineoplastic activity of the original polypeptide when expressed in an engineered immune cell. These chemical modifications include, but are not limited to, substituting alternative R groups and varying the degree of saturation at specific carbon atoms of a reference polypeptide. The protein analogs can be relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding GLUT5 can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

In some embodiments, a nucleic acid as disclosed herein further comprises a regulatory sequence directing the expression of the GLUT5 gene and any receptor disclosed herein. In further embodiments, the nucleic acid comprises a single regulatory sequence directing the expression of both of the GLUT5 gene and the receptor. In other embodiments, the nucleic acid comprises a first regulatory sequence directing the expression of the GLUT5 gene and a second regulatory sequence directing the expression of the receptor. In other embodiments, the first regulatory sequence is the same as the second regulatory sequence. In some embodiments, the first regulatory sequence is different from the second regulatory sequence.

### Vectors

Many expression vectors are available and known to those of skill in the art and can be used for nonendogenous expression of GLUT5. The choice of expression vector will be influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells.

Vectors also can contain additional nucleotide sequences operably linked to the ligated nucleic acid molecule, such as, for example, an epitope tag such as for localization, e.g., a hexa-his tag (SEQ ID NO: 41) or a myc tag, hemagglutinin tag or a tag for purification, for example, a GST fusion, and a sequence for directing protein secretion and/or membrane association.

Expression of the heterologous GLUT5 gene can be controlled by any promoter/enhancer known in the art. Suitable bacterial promoters are well known in the art and described herein below. Other suitable promoters for mammalian cells, yeast cells and insect cells are well known in the art and some are exemplified below. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bernoist and Chambon, Nature 290:304-310(1981)), the promoter contained in the 3′ long terminal repeat of Rous sarcoma virus (Yamamoto et al., Cell 22:787-797(1980)), the herpes thymidine kinase promoter (Wagner et al., Proc. Natl. Acad. Sci. USA 75: 1441-1445 (1981)), the regulatory sequences of the metallothionein gene (Brinster et al., Nature 296:39-42 (1982)); prokaryotic expression vectors such as the β-lactamase promoter (Jay et al., Proc. Natl. Acad. Sci. USA 75:5543 (1981)) or the tac promoter (DeBoer et al., Proc. Natl. Acad. Sci. USA 50:21-25(1983)); see also “Useful Proteins from Recombinant Bacteria”: in Scientific American 242:79-94 (1980)); plant expression vectors containing the nopaline synthetase promoter (Herrera-Estrella et al., Nature 505:209-213(1984)) or the cauliflower mosaic virus 35S RNA promoter (Gardner et al., Nucleic Acids Res. 9:2871(1981)), and the promoter of the photosynthetic enzyme ribulose bisphosphate carboxylase (Herrera-Estrella et al., Nature 510: 115-120(1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 55:639-646 (1984); Ornitz et al., Cold Spring Harbor Symp. Quant. Biol. 50:399-409(1986); MacDonald, Hepatology 7:425-515 (1987)); insulin gene control region which is active in pancreatic beta cells (Hanahan et al., Nature 515: 115-122 (1985)), immunoglobulin gene control region which is active in lymphoid cells (Grosschedl et al., Cell 55:647-658 (1984); Adams et al., Nature 515:533-538 (1985); Alexander et al., Mol. Cell Biol. 7: 1436-1444 (1987)), mouse mammary tumor virus control region which is active in testicular, breast, lymphoid and mast cells (Leder et al., Cell 15:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-403 (1985)); Hammer et al., Science 255:53-58 (1987)), alpha-1 antitrypsin gene control region which is active in liver (Kelsey et al., Genes and Devel. 7:161-171 (1987)), beta globin gene control region which is active in myeloid cells (Magram et al., Nature 515:338-340 (1985)); Kollias et al., Cell 5:89-94 (1986)), myelin basic protein gene control region which is active in oligodendrocyte cells of the brain (Readhead et al., Cell 15:703-712 (1987)), myosin light chain-2 gene control region which is active in skeletal muscle (Shani, Nature 514:283-286 (1985)), and gonadotrophic releasing hormone gene control region which is active in gonadotrophs of the hypothalamus (Mason et al., Science 254: 1372-1378 (1986)).

In addition to the promoter, the expression vector typically contains a transcription unit or expression cassette that contains all the additional elements required for the expression of an antibody, or antigen binding fragment thereof, in host cells. A typical expression cassette contains a promoter operably linked to the nucleic acid sequence encoding the polypeptide chains of interest and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a germline antibody chain under the direction of the polyhedron promoter or other strong baculovirus promoter.

Any methods known to those of skill in the art for the insertion of DNA fragments into a vector can be used to construct expression vectors containing a nucleic acid encoding any of the polypeptides provided herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

Exemplary plasmid vectors useful to produce the polypeptides provided herein contain a strong promoter, such as the HCMV immediate early enhancer/promoter or the MHC class I promoter, an intron to enhance processing of the transcript, such as the HCMV immediate early gene intron A, and a polyadenylation (poly A) signal, such as the late SV40 polyA signal.

Genetic modification of engineered immune cells (e.g., T cells, NK cells) can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding GLUT5 can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide GLUT5 overexpressing cells, a retroviral vector can be employed for transduction. However, any other suitable viral vector or non-viral delivery system can be used for genetic modification of cells. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller et al. (1985) Mol. Cell. Biol. 5:431-437); PA317 (Miller et al. (1986) Mol. Cell. Biol. 6:2895-2902); and CRIP (Danos et al. (1988) Proc. Natl. Acad. Sci. USA 85:6460-6464). Non-amphotropic particles are suitable too, e.g., particles pseudotyped with VSVG, RD 114 or GALV envelope and any other known in the art.

Possible methods of transduction also include direct co-culture of the cells with producer cells, e.g., by the method of Bregni, et al., Blood 80: 1418-1422(1992), or culturing with viral supernatant alone or concentrated vector stocks with or without appropriate growth factors and polycations, e.g., by the method of Xu, et al., Exp. Hemat. 22:223-230 (1994); and Hughes, et al., J. Clin. Invest. 89: 1817 (1992).

Transducing viral vectors can be used to express a co-stimulatory ligand and/or secretes a cytokine (e.g., 4-1BBL and/or IL-12) in an engineered immune cell. In some embodiments, the chosen vector exhibits high efficiency of infection and stable integration and expression (see, e.g., Cayouette et al., Human Gene Therapy 8:423-430 (1997); Kido et al., Current Eye Research 15:833-844 (1996); Bloomer et al., Journal of Virology 71:6641-6649, 1997; Naldini et al., Science 272:263 267 (1996); and Miyoshi et al., Proc. Natl. Acad. Sci. U.S.A. 94: 10319, (1997)). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, (1990); Friedman, Science 244: 1275-1281 (1989); Eglitis et al., BioTechniques 6:608-614, (1988); Tolstoshev et al., Current Opinion in Biotechnology 1:55-61(1990); Sharp, The Lancet 337: 1277-1278 (1991); Cornetta et al., Nucleic Acid Research and Molecular Biology 36:311-322 (1987); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med 323:370 (1990); Anderson et al., U.S. Pat. No. 5,399,346).

In certain non-limiting embodiments, the vector expressing GLUT5 is a retroviral vector, e.g., an oncoretroviral vector. In some instances, the retroviral vector is a SFG retroviral vector or murine stem cell virus (MSCV) retroviral vector. In certain non-limiting embodiments, the vector expressing a GLUT5 nucleic acid sequence is a lentiviral vector. In certain non-limiting embodiments, the vector expressing a GLUT5 nucleic acid sequence is a transposon vector.

Non-viral approaches can also be employed for the expression of a protein in a cell. For example, a nucleic acid molecule can be introduced into a cell by administering the nucleic acid in the presence of lipofection (Feigner et al., Proc. Nat'l. Acad. Sci. U.S.A. 84:7413, (1987); Ono et al., Neuroscience Letters 17:259 (1990); Brigham et al., Am. J. Med. Sci. 298:278, (1989); Staubinger et al., Methods in Enzymology 101:512 (1983)), asialoorosomucoid-polylysine conjugation (Wu et al., Journal of Biological Chemistry 263 14621 (1988); Wu et al., Journal of Biological Chemistry 264: 16985 (1989)), or by micro-injection under surgical conditions (Wolff et al., Science 247: 1465 (1990)). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

In some embodiments, a vector as disclosed herein further comprises a regulatory sequence directing the expression of the GLUT5 gene and any receptor disclosed herein. In further embodiments, the vector comprises a single regulatory sequence directing the expression of both of the GLUT5 gene and the receptor. In other embodiments, the vector comprises a first regulatory sequence directing the expression of the GLUT5 gene and a second regulatory sequence directing the expression of the receptor. In other embodiments, the first regulatory sequence is the same as the second regulatory sequence. In some embodiments, the first regulatory sequence is different from the second regulatory sequence.

### Engineered Immune Cells

The presently disclosed subject matter provides engineered immune cells that overexpress GLUT5. In some embodiments, the engineered immune cells may further comprise an engineered receptor (e.g., a CAR, caTCR, or eTCR) or other ligand that comprises an extracellular antigen-binding domain, a transmembrane domain and an intracellular domain, where the extracellular antigen-binding domain specifically binds a tumor antigen, including a tumor receptor or ligand. In certain embodiments, immune cells can be transduced with a vector comprising nucleic acid sequences that encode GLUT5.

Examples of tumor antigens include, but are not limited to, 5T4, alpha 5β1-integrin, 707-AP, A33, AFP, ART-4, B7H4, BAGE, Bcl-2, β-catenin, BCMA, Bcr-abl, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC16, MUM-1-B, myc, MUM-2, MUM-3, NA88-A, NYESO-1, NY-Eso-B, p53, proteinase-3, p190 minor bcr-abl, Pml/RARα, PRAME, progesterone receptor, PSA, PSCA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, or WT1

The presently disclosed subject matter also provides methods of using such cells for the treatment of a tumor. The engineered immune cells of the presently disclosed subject matter can be cells of the lymphoid lineage or myeloid lineage. Examples of myeloid cells include but are not limited to, mast cells, monocytes, macrophages, dendritic cells, eosinophils, neutrophils, basophils. The lymphoid lineage, comprising B, T, and natural killer (NK) cells, provides for the production of antibodies, regulation of the cellular immune system, detection of foreign agents in the blood, detection of cells foreign to the host, and the like. Non-limiting examples of immune cells of the lymphoid lineage include T cells, Natural Killer (NK) cells, embryonic stem cells, and pluripotent stem cells (e.g., those from which lymphoid cells can be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., T_{EM} cells and TEMRA cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells.

Additionally or alternatively, in some embodiments, the engineered immune cell is a T cell lacking the expression of a cytokine (such as TNFα), or T-Cell-Specific Transcription Factor (TCF-1), or both.

Natural killer (NK) cells can be lymphocytes that are part of cell-mediated immunity and act during the innate immune response. NK cells do not require prior activation in order to perform their cytotoxic effect on target cells.

The engineered immune cells of the presently disclosed subject matter can express non-endogenous levels of GLUT5 for the treatment of cancer, e.g., for treatment of tumor. Such engineered immune cells can be administered to a subject (e.g., a human subject) in need thereof for the treatment of cancer. In some embodiments, the immune cell is a lymphocyte, such as a T cell, a B cell or a natural killer (NK) cell. In certain embodiments, the engineered immune cell is a T cell. The T cell can be a CD4⁺ T cell or a CD8⁺ T cell. In certain embodiments, the T cell is a CD4⁺ T cell. In certain embodiments, the T cell is a CD8⁺ T cell.

The presently disclosed engineered immune cells of the present technology may further include at least one recombinant or exogenous co-stimulatory ligand. For example, the presently disclosed engineered immune cells can be further transduced with at least one co-stimulatory ligand, such that the engineered immune cells co-expresses or is induced to co-express GLUT5 and the at least one co-stimulatory ligand. Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFP)/lymphotoxin-alpha (LTa), lymphotoxin-beta O-Tβ), CD257/B cell-activating factor (B AFF)/Bly s/THANK/Tall-1, glucocorticoid-induced TNF Receptor ligand (GITRL), and T F-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the engineered immune cell comprises one recombinant co-stimulatory ligand (e.g., 4-1BBL). In certain embodiments, the engineered immune cell comprises two recombinant co-stimulatory ligands (e.g., 4-1BBL and CD80). CARs comprising at least one co-stimulatory ligand are described in U.S. Pat. No. 8,389,282, which is incorporated by reference in its entirety.

Furthermore, the presently disclosed engineered immune cells can further comprise at least one exogenous cytokine. For example, a presently disclosed engineered immune cell can be further transduced with at least one cytokine, such that the engineered immune cells secrete the at least one cytokine as well as express GLUT5. In certain embodiments, the at least one cytokine is selected from the group consisting of IL-2, IL-3, IL-6, IL-7, IL-11, IL-12, IL-15, IL-17, and IL-21. In certain embodiments, the cytokine is IL-12.

The engineered immune cells can be generated from peripheral donor lymphocytes, e.g., those disclosed in Sadelain, M., et al., Nat Rev Cancer 3:35-45 (2003), in Morgan, R. A. et al. (2006) Science 314: 126-129, in Panelli et al. (2000) J Immunol 164:495-504; Panelli et al. (2000) J Immunol 164:4382-4392 (2000), and in Dupont et al. (2005) Cancer Res 65:5417-5427; Papanicolaou et al. (2003) Blood 102:2498-2505. The engineered immune cells (e.g., T cells) can be autologous, non-autologous (e.g., allogeneic), or derived in vitro from engineered progenitor or stem cells.

In certain embodiments, the presently disclosed engineered immune cells (e.g., T cells) expresses from about 1 to about 5, from about 1 to about 4, from about 2 to about 5, from about 2 to about 4, from about 3 to about 5, from about 3 to about 4, from about 4 to about 5, from about 1 to about 2, from about 2 to about 3, from about 3 to about 4, or from about 4 to about 5 vector copy numbers per cell of a GLUT5 heterologous nucleic acid.

For example, the higher the non-endogenous levels of GLUT5 in an engineered immune cell, the greater cytotoxicity and/or cytokine production the engineered immune cell exhibits. Additionally, or alternatively, the cytotoxicity and cytokine production of a presently disclosed engineered immune cell (e.g., T cell) are proportional to the expression level of GLUT5 in the immune cell.

The unpurified source of immune cells can be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-immune cell initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. In some embodiments, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). In some embodiments, the cells are collected in a medium comprising 2% fetal calf serum (FCS) or 0.2% bovine serum albumin (BSA) or any other suitable, preferably sterile, isotonic medium.

In some embodiments, the engineered immune cells comprise one or more additional modifications. For example, in some embodiments, the engineered immune cells comprise and express (is transduced to express) a chimeric co-stimulatory receptor (CCR). CCR is described in Krause et al. (1998) J. Exp. Med. 188(4):619-626, and US20020018783, the contents of which are incorporated by reference in their entireties. CCRs mimic co-stimulatory signals, but unlike, engineered receptors, do not provide a T-cell activation signal, e.g., CCRs lack a CD3ζ polypeptide. CCRs provide co-stimulation, e.g., a CD28-like signal, in the absence of the natural co-stimulatory ligand on the antigen-presenting cell. A combinatorial antigen recognition, i.e., use of a CCR in combination with an engineered receptor, can augment T-cell reactivity against the dual-antigen expressing T cells, thereby improving selective tumor targeting.

In some embodiments, the engineered immune cells are further modified to suppress expression of one or more genes. In some embodiments, the engineered immune cells are further modified via genome editing. Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, for example, U.S. Pat. Nos. 7,888,121; 7,972,854; 7,914,796; 7,951,925; 8,110,379; 8,409,861; 8,586,526; U.S. Patent Publications 20030232410; 20050208489; 20050026157; 20050064474; 20060063231; 201000218264; 20120017290; 20110265198; 20130137104; 20130122591; 20130177983 and 20130177960, the disclosures of which are incorporated by reference in their entireties. These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knock out of a gene or the insertion of a sequence of interest (targeted integration). Cleavage can occur through the use of specific nucleases such as engineered zinc finger nucleases (ZFN), transcription-activator like effector nucleases (TALENs), or using the CRISPR/Cas system with an engineered crRNA/tracr RNA (‘single guide RNA’) to guide specific cleavage. In some embodiments, the engineered immune cells are modified to disrupt or reduce expression of an endogenous T-cell receptor gene (see, e.g. WO 2014153470, which is incorporated by reference in its entirety). In some embodiments, the engineered immune cells are modified to result in disruption or inhibition of PD1, PDL-1 or CTLA-4 (see, e.g. U.S. Patent Publication 20140120622), or other immunosuppressive factors known in the art (Wu et al. (2015) Oncoimmunology 4(7): e1016700, Mahoney et al. (2015) Nature Reviews Drug Discovery 14, 561-584).

### Administration

Engineered immune cells overexpressing GLUT5 of the presently disclosed subject matter can be provided systemically or directly to a subject for treating cancer. In certain embodiments, engineered immune cells are directly injected into an organ of interest. Additionally or alternatively, the engineered immune cells are provided indirectly to the organ of interest, for example, by administration into the circulatory system (e.g., the tumor vasculature) or into the tissue of interest (e.g., solid tumor). Expansion and differentiation agents can be provided prior to, during or after administration of cells and compositions to increase production of the engineered immune cells either in vitro or in vivo.

Engineered immune cells of the presently disclosed subject matter can be administered in any physiologically acceptable vehicle, systemically or regionally, normally intravascularly, intraperitoneally, intrathecally, or intrapleurally, although they may also be introduced into bone or other convenient site where the cells may find an appropriate site for regeneration and differentiation (e.g., thymus). In certain embodiments, at least 1×10⁵ cells can be administered, eventually reaching 1×10¹⁰ or more. In certain embodiments, at least 1×10⁶ cells can be administered. A cell population comprising engineered immune cells can comprise a purified population of cells. Those skilled in the art can readily determine the percentage of engineered immune cells in a cell population using various well-known methods, such as fluorescence activated cell sorting (FACS). The ranges of purity in cell populations comprising engineered immune cells can be from about 50% to about 55%, from about 55% to about 60%, about 60% to about 65%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The engineered immune cells can be introduced by injection, catheter, or the like. If desired, factors can also be included, including, but not limited to, interleukins, e.g., IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

In certain embodiments, compositions of the presently disclosed subject matter comprise pharmaceutical compositions comprising engineered immune cells overexpressing GLUT5 with a pharmaceutically acceptable carrier. Administration can be autologous or non-autologous. For example, engineered immune cells overexpressing GLUT5 and compositions comprising the same can be obtained from one subject, and administered to the same subject or a different, compatible subject. Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising engineered immune cells overexpressing GLUT5), it can be formulated in a unit dosage injectable form (solution, suspension, emulsion).

### Formulations

Engineered immune cells over-expressing GLUT5 and compositions comprising the same can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter, e.g., a composition comprising engineered immune cells, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the engineered immune cells of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is suitable particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the engineered immune cells as described in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the engineered immune cells of the presently disclosed subject matter is the quantity of cells necessary to achieve an optimal effect. The quantity of cells to be administered will vary for the subject being treated. In certain embodiments, from about 10² to about 10¹², from about 10³ to about 10¹¹, from about 10⁴ to about 10¹⁰, from about 10⁵ to about 10⁹, or from about 10⁶ to about 10⁸ engineered immune cells of the presently disclosed subject matter are administered to a subject. More effective cells may be administered in even smaller numbers. In some embodiments, at least about 1×10⁸, about 2×10⁸, about 3×10⁸, about 4×10⁸, about 5×10⁸, about 1×10⁹, about 5×10⁹, about 1×10¹⁰, about 5×10¹⁰, about 1×10¹¹, about 5×10¹¹, about 1×10¹² or more engineered immune cells of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art. Generally, engineered immune cells are administered at doses that are nontoxic or tolerable to the patient.

The skilled artisan can readily determine the amount of cells and optional additives, vehicles, and/or carrier in compositions to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Therapeutic Uses of the Engineered Immune Cells of the Present Technology

For treatment, the amount of the engineered immune cells provided herein administered is an amount effective in producing the desired effect, for example, treatment of a cancer or one or more symptoms of a cancer. An effective amount can be provided in one or a series of administrations of the engineered immune cells provided herein. An effective amount can be provided in a bolus or by continuous perfusion. For adoptive immunotherapy using antigen-specific T cells, cell doses in the range of about 10⁶ to about 10¹⁰ are typically infused. Lower doses of the engineered immune cells may be administered, e.g., about 10⁴ to about 10⁸.

The engineered immune cells of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the engineered immune cells and the compositions comprising thereof are intravenously administered to the subject in need. Methods for administering cells for adoptive cell therapies, including, for example, donor lymphocyte infusion and engineered T cell therapies, and regimens for administration are known in the art and can be employed for administration of the engineered immune cells provided herein.

For example, the presently disclosed subject matter provides methods of reducing tumor burden in a subject. In one non-limiting example, the method of reducing tumor burden comprises administering an effective amount of the presently disclosed engineered immune cells to the subject and administering a suitable antibody targeted to the tumor, thereby inducing tumor cell death in the subject. In some embodiments, the engineered immune cells and the antibody are administered at different times. For example, in some embodiments, the engineered immune cells are administered and then the antibody is administered. In some embodiments, the antibody is administered 1 hour, 2 hours, 3 hours, 4 hours, 5 hours, 6 hours, 7 hours, 8 hours, 9 hours, 10 hours, 11 hours, 12 hours, 18 hours, 24 hours, 30 hours, 26 hours, 48 hours or more than 48 hours after the administration of the engineered immune cells of the present technology.

The presently disclosed subject matter provides various methods of using the engineered immune cells (e.g., T cells) provided herein, overexpressing GLUT5. In some embodiments, the engineered immune cell is a CAR, caTCR, or eTCR. For example, the presently disclosed subject matter provides methods of reducing tumor burden in a subject. In one non-limiting example, the method of reducing tumor burden comprises administering an effective amount of the presently disclosed engineered immune cells to the subject, thereby inducing tumor cell death in the subject.

The presently disclosed engineered immune cells can reduce the number of tumor cells, reduce tumor size, and/or eradicate the tumor in the subject. In certain embodiments, the method of reducing tumor burden comprises administering an effective amount of engineered immune cells to the subject, thereby inducing tumor cell death in the subject. Non-limiting examples of suitable tumors include adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof. In some embodiments, the cancer is a relapsed or refractory cancer. In some embodiments, the cancer is resistant to one or more cancer therapies, e.g., one or more chemotherapeutic drugs.

The presently disclosed subject matter also provides methods of increasing or lengthening survival of a subject with cancer (e.g., a tumor). In one non-limiting example, the method of increasing or lengthening survival of a subject with cancer (e.g., a tumor) comprises administering an effective amount of the presently disclosed engineered immune cell to the subject, thereby increasing or lengthening survival of the subject. The presently disclosed subject matter further provides methods for treating or preventing cancer (e.g., a tumor) in a subject, comprising administering the presently disclosed engineered immune cells to the subject. Also provided herein are methods for treating of inhibiting tumor growth or metastasis in a subject comprising contacting a tumor cell with an effective amount of any of the engineered immune cells provided herein.

Cancers whose growth may be inhibited using the engineered immune cells of the presently disclosed subject matter include cancers typically responsive to immunotherapy. Non-limiting examples of cancers for treatment include breast cancer, endometrial cancer, ovarian cancer, colon cancer, lung cancer, stomach cancer, prostate cancer, renal cancer, pancreatic cancer, brain cancer, acute lymphoblastic leukemia (ALL), chronic lymphocytic leukemia (CLL), acute myeloid leukemia (AML), and metastases thereof.

Additionally, the presently disclosed subject matter provides methods of increasing immune-activating cytokine production in response to a cancer cell in a subject in need thereof. In one non-limiting example, the method comprises administering the presently disclosed engineered immune cell to the subject. The immune-activating cytokine (which is also referred to herein as a cytokine) can be granulocyte macrophage colony stimulating factor (GM-CSF), IFNα, IFN-β, IFN-δ, TNFα, IL-2, IL-3, IL-6, IL-11, IL-7, IL-12, IL-15, IL-21, interferon regulatory factor 7 (IRF7), and combinations thereof. In certain embodiments, the GLUT5 overexpressing engineered immune cells of the presently disclosed subject matter increase the production of GM-CSF, IFN-7, and/or TNFα.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in the presently disclosed subject matter is administered to these subjects to elicit an anti-tumor response, with the objective of palliating their condition. Ideally, reduction in tumor mass occurs as a result, but any clinical improvement constitutes a benefit. Clinical improvement comprises decreased risk or rate of progression or reduction in pathological consequences of the tumor.

Another group of suitable subjects is known in the art as the “adjuvant group.” These are individuals who have had a history of neoplasia, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes. Another group has a genetic predisposition to neoplasia but has not yet evidenced clinical signs of neoplasia. For instance, women testing positive for a genetic mutation associated with breast cancer, but still of childbearing age, can wish to receive one or more of the engineered immune cells described herein in treatment prophylactically to prevent the occurrence of neoplasia until it is suitable to perform preventive surgery.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the GLUT5 over-expressing engineered immune cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. Modification of the engineered immune cells can include engineering a suicide gene into the GLUT5 over-expressing T cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). The suicide gene can be included within the vector comprising nucleic acids encoding GLUT5. A presently disclosed engineered immune cell (e.g., a T cell) incorporated with a suicide gene can be pre-emptively eliminated at a given time point post T cell infusion, or eradicated at the earliest signs of toxicity.

### Combination Therapy

The compositions of the present technology may be employed in conjunction with other therapeutic agents useful in the treatment of cancers. For example, the GLUT5 over-expressing engineered immune cells of the present technology may be separately, sequentially or simultaneously administered with at least one additional cancer therapy. In some embodiments, the additional cancer therapy is selected from among a chemotherapy, a radiation therapy, an immunotherapy, a monoclonal antibody, an anti-cancer nucleic acid, an anti-cancer protein, an anti-cancer virus or microorganism, a cytokine, or any combination thereof.

Radiation therapy includes, but is not limited to, exposure to radiation, e.g., ionizing radiation, UV radiation, as known in the art. Exemplary dosages include, but are not limited to, a dose of ionizing radiation at a range from at least about 2 Gy to not more than about 10 Gy or a dose of ultraviolet radiation at a range from at least about 5 J/m² to not more than about 50 J/m², usually about 10 J/m².

In some embodiments, the methods further comprise sequentially, separately, or simultaneously administering an immunotherapy to the subject. In some embodiments, the immunotherapy regulates immune checkpoints. In further embodiments, the immunotherapy comprises, or consists essentially of, or yet further consists of an immune checkpoint inhibitor, such as an Cytotoxic T-Lymphocyte Associated Protein 4 (CTLA4) inhibitor, or a Programmed Cell Death 1 (PD-1) inhibitor, or a Programmed Death Ligand 1 (PD-L1) inhibitor. In yet further embodiments, the immune checkpoint inhibitor comprises, or consists essentially of, or yet further consists of an antibody or an equivalent thereof recognizing and binding to an immune checkpoint protein, such as an antibody or an equivalent thereof recognizing and binding to CTLA4 (for example, Yervoy (ipilimumab), CP-675,206 (tremelimumab), AK104 (cadonilimab), or AGEN1884 (zalifrelimab)), or an antibody or an equivalent thereof recognizing and binding to PD-1 (for example, Keytruda (pembrolizumab), Opdivo (nivolumab), Libtayo (cemiplimab), Tyvyt (sintilimab), BGB-A317 (tislelizumab), JS001 (toripalimab), SHR1210 (camrelizumab), GB226 (geptanolimab), JS001 (toripalimab), AB122 (zimberelimab), AK105 (penpulimab), HLX10 (serplulimab), BCD-100 (prolgolimab), AGEN2034 (balstilimab), MGA012 (retifanlimab), AK104 (cadonilimab), HX008 (pucotenlimab), PF-06801591 (sasanlimab), JNJ-63723283 (cetrelimab), MGD013 (tebotelimab), CT-011 (pidilizumab), or Jemperli (dostarlimab)), or an antibody or an equivalent thereof recognizing and binding to PD-L1 (for example, Tecentriq (atezolizumab), Imfinzi (durvalumab), Bavencio (avelumab), CS1001 (sugemalimab), or KN035 (envafolimab)).

In some embodiments, the methods further comprise sequentially, separately, or simultaneously administering a cytokine to the subject. In some embodiments, the cytokine is administered prior to, during, or subsequent to administration of the one or more engineered immune cells. In some embodiments, the cytokine is selected from the group consisting of interferon α, interferon β, interferon γ, complement C5a, IL-2, TNFα, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3C_{L}1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

The methods for treating cancer may further comprise sequentially, separately, or simultaneously administering to the subject at least one chemotherapeutic agent, optionally selected from the group consisting of nitrogen mustards, ethylenimine derivatives, alkyl sulfonates, nitrosoureas, gemcitabine, triazenes, folic acid analogs, anthracyclines, taxanes, COX-2 inhibitors, pyrimidine analogs, purine analogs, antibiotics, enzyme inhibitors, epipodophyllotoxins, platinum coordination complexes, vinca alkaloids, substituted ureas, methyl hydrazine derivatives, adrenocortical suppressants, hormone antagonists, endostatin, taxols, camptothecins, SN-38, doxorubicin, doxorubicin analogs, antimetabolites, alkylating agents, antimitotics, anti-angiogenic agents, tyrosine kinase inhibitors, mTOR inhibitors, heat shock protein (HSP90) inhibitors, proteosome inhibitors, HDAC inhibitors, pro-apoptotic agents, methotrexate and CPT-11.

Additionally or alternatively, in some embodiments, the method further comprises sequentially, separately, or simultaneously administering to the recipient subject any one or more of: a fructose; a pyruvate kinase M2 (PKM2) activator (such as DASA58); or a ketohexokinase (KHK) inhibitor (such as 6-(4-(2-Hydroxyethyl)piperazin-1-yl)-2-(3-(hydroxymethyl)-piperidin-1-yl)-4-(trifluoromethyl)nicotinonitrile). Non-limiting examples of PKM2 activators include TEPP-46, AG 348, or DASA-58.

TEPP-46 is a cell-permeable thienopyrrolopyridazinone that exhibits good aqueous solubility (29.6 μg/mL or 79.5 μM in PBS, pH 7.4) and acts as a potent allosteric PKM2 activator (AC₅₀=92 nM; [PKM2]=0.1 nM) by stabilizing PKM2 tetramer via stoichiometric interaction with FBP-bound PKM2 (2 activator/4 FBP/tetramer) in a similar manner as PKM2 Activator III (Cat. No. 504537). Its CAS number is 1221186-53-3 and has a structure as shown below. TEPP-46 is available from Millipore Sigma with Catalog No. 5.05487.0001.

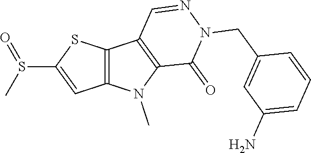 [SMILES: Cn1c2cc(S(C)=O)sc2c2cnn(Cc3cccc(N)c3)c(=O)c21]

DASA-58 (the structure of which is listed below) is an selective activator of pyruvate kinase M2 (PKM2) with an AC₉₀ value of 680 nM, and an AC₅₀ value of 38 nM. Its CAS number is 1203494-49-8 and has a structure as shown below. It is available from APExBIO with Catalog No. B6035.

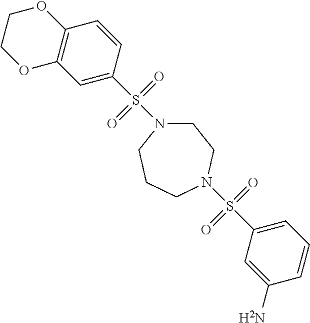 [SMILES: [2NH2]c1cccc(S(=O)(=O)N2CCCN(S(=O)(=O)c3ccc4c(c3)OCCO4)CC2)c1]

AG 348 is an allosteric activator of red blood cell pyruvate kinase, increases enzymatic activity, protein stability, and ATP levels over a broad range of PKLR genotypes. Its CAS number is 1260075-17-9 and has a structure as shown below. It is available from ProbeChem with Catalog No. PC-43151.

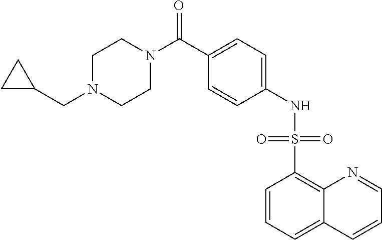 [SMILES: O=C(c1ccc(NS(=O)(=O)c2cccc3cccnc23)cc1)N1CCN(CC2CC2)CC1]

An inhibitor of KHK decreases, slows down, or partically or completely inhibits the conversion of fructose to fructose-1-phosphate. Non-limiting examples of the KHK inhibitors include 6-(4-(2-Hydroxyethyl)piperazin-1-yl)-2-(3-(hydroxymethyl)-piperidin-1-yl)-4-(trifluoromethyl)nicotinonitrile) and PF-06835919 (2-((1R,5S,6R)-3-(2-((S)-2-Methylazetidin-1-yl)-6-(trifluoromethyl)pyrimidin-4-yl)-3-azabicyclo[3.1.0]hexan-6-yl)acetic acid).

PF-06835919 (2-((1R,5S,6R)-3-(2-((S)-2-Methylazetidin-1-yl)-6-(trifluoromethyl)pyrimidin-4-yl)-3-azabicyclo[3.1.0]hexan-6-yl)acetic acid) is a ketohexokinase (KHK) inhibitor developed by Pfizer. Its CAS number is 2102501-84-6 and has a structure as shown below. It is available from MedKoo Biosciences, Inc. with Catalog No. 555163.

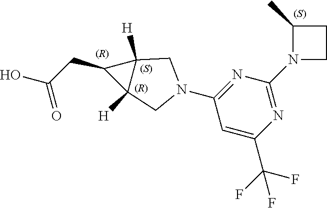

### Kits

The presently disclosed subject matter provides kits for the treatment or prevention of a disease, such as cancer. In certain embodiments, the kit comprises a therapeutic or prophylactic composition containing an effective amount of an engineered immune cell comprising a vector that overexpresses GLUT5. In some embodiments, the engineered immune cell is a CAR, caTCR, or eTCR. In particular embodiments, the engineered immune cell further expresses at least one co-stimulatory ligand.

In some embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the engineered immune cell can be provided together with instructions for administering the engineered immune cell to a subject having or at risk of developing cancer. The instructions will generally include information about the use of the composition for the treatment or prevention of cancer. In other embodiments, the instructions include at least one of the following: description of the therapeutic agent; dosage schedule and administration for treatment or prevention of cancer or symptoms thereof; precautions; warnings; indications; counter-indications; overdose information; adverse reactions; animal pharmacology; clinical studies; and/or references. The instructions may be printed directly on the container (when present), or as a label applied to the container, or as a separate sheet, pamphlet, card, or folder supplied in or with the container.

In some embodiments, the at least one engineered immune cell of the present technology utilized GLUT5 and binds to target cells that express a target antigen on the cell surface. The at least one engineered immune cell of the present technology may be provided in the form of a prefilled syringe or autoinjection pen containing a sterile, liquid formulation or lyophilized preparation (e.g., Kivitz et al., Clin. Ther. 28:1619-29 (2006)).

A device capable of delivering the kit components through an administrative route may be included. Examples of such devices include syringes (for parenteral administration) or inhalation devices.

The kit components may be packaged together or separated into two or more containers. In some embodiments, the containers may be vials that contain sterile, lyophilized formulations of engineered immune cell composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers.

Also provided herein are kits for use in the manufacture of an engineered immune cell that overexpresses GLUT5. In certain embodiments, the kit comprises a vector comprising a heterologous GLUT5 nucleic acid. Additionally or alternatively, in some embodiments, the kit further comprises a vector comprising an engineered T-cell receptor (TCR) or other cell-surface ligand that binds to a target antigen, such as a tumor antigen or viral protein. In some embodiments, the vector comprising the heterologous GLUT5 nucleic acid and the vector comprising the engineered T-cell receptor (TCR) or cell-surface ligand that binds to a target antigen are the same. In other embodiments, the vector comprising the heterologous GLUT5 nucleic acid and the vector comprising the engineered T-cell receptor (TCR) or cell-surface ligand that binds to a target antigen are distinct. Additionally or alternatively, in some embodiments, the kits further comprise fructose, a pyruvate kinase M2 (PKM2) activator, a ketohexokinase (KHK) inhibitor, or an additional anti-cancer therapeutic agent.

### EXAMPLES

Current strategies to enhance the effects of immunotherapy have focused on modulating the tumor microenvironment (TME) and more specially the metabolism of tumors in order to overcome limited efficacy. The examples herein argue for fundamentally enhancing T cells by providing them with a unique privileged nutrient source that reprograms their metabolism and allow them to overcome the limitations of the harsh TME.

Enhancing nutrient transport in T cells by overexpressing GLUT5: Literature precedent implicates a lack of nutrients as one of the causes of reduced T cells in the TME. It is demonstrated herein that both acute and chronically stimulated T cells are unable to adequately proliferate in low glucose conditions. Moreover, using leukemia cells as a model system, it is demonstrated herein that in low glucose conditions, cells can readily shunt carbons through the serine synthesis pathway, and that this can be reversed by overexpressing GLUT5 in the presence of fructose. It is further investigated herein that nutrient transporter modulation can enhance the effectiveness of T cells, linking metabolic engineering to immunotherapy.

Develop novel strategies to trace GLUT5-mediated metabolism in vitro and in vivo using isotope tracing and hyperpolarized MRI. In order to characterize early response to therapy as well as fully understand the implications of metabolic reprogramming, in vivo methods are necessary to establish metabolic biomarkers (Di Gialleonardo et al. Cancer Res 77, 3113-3120 (2017); Dong et al. Cancer Res 79, 242-250 (2019); Day et al. Nat Med 13, 1382-1387 (2007); Ward et al. Cancer Res 70, 1296-1305 (2010)). With this in mind, two strategies are developed for interrogating the effects of fructose transporter overexpression. First, stable isotope tracing mass spectroscopy is utilized, validating in vitro and in bolus injections in vivo with infusion of modified T cells. By pulling down T cells and rapidly extracting them, it is invegated that the newfound ability of T cells metabolizes fructose in vivo. Furthermore, a non-invasive approach is developed to image enhanced fructose metabolism using hyperpolarized ¹³C fructose MRI. Leveraging the development of a hyperpolarized microNMR system (Jeong et al. Sci Adv 3, e1700341 (2017)) fructose flux is measured in small numbers of T cells and mice are imaged with infusion of T cells. This provides a method for translating this approach from mice to humans with potential T-cell therapies.

Demonstrate that metabolism can be reversed in the TME, leveraging the understanding of nutrient availability: By overexpressing a unique nutrient transporter system that feeds into glycolysis while providing a nutrient that tumor cells do not readily use (due to low expression of the fructose transport system), it is investigated for the first time that availability of intracellular carbohydrate sources can reverse T-cell metabolism. Additionally, the flux of these metabolites are traced into metabolic intermediates, developing a strategy for understanding immunometabolism in vivo for applications to other modulations.

Execute a therapeutic strategy utilizing supplementation of a naturally occurring sugar, fructose: Given that this approach is taken in vivo, an optimal strategy is developed to modulate the TME using fructose supplementation. Typically, most dietary fructose is taken up by the liver and small intestines, given their high levels of ketohexokinase (KHK). In these examples, bolus injections are combined with KHK inhibition and an optimal strategy is developed for raising tumor fructose, ultimately aiming to enhance immunotherapy.

### Example 1: Approaches and Corresponding Experimental Data

Ensuring robust and unbiased results: the strategy disclosed herein is designed to guarantee robust, unbiased, and statistically accurate data. For this purpose, data analysis is automated wherever possible. Data analysis is performed in concert with the Department of Biostatistics at Memorial Sloan Kettering cancer Center (MSK), and results are only be considered statistically significant if confidence levels are >95%.

Consideration of relevant biological variables: Since this study is exclusively geared toward modulating T cells and using iotope tracing and hyperpolarized MRI to study immunometabolism, controls in every case are used for both drug vehicle and wild type T cells.

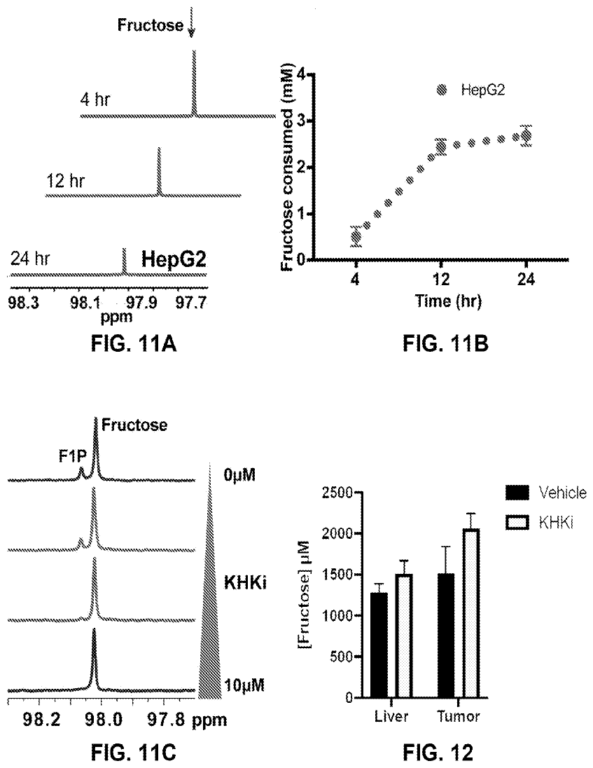

Data: The data measuring fructose flux in vitro is presented herein, which (1) demonstrates the role and modulation of fructose flux in glycolysis, (2) provides an extensively characterized model of T-cell exhaustion for use in metabolic studies, (3) demonstrates GLUT5 overexpression in primary human T cells, and (4) provides methods for tracing fructose metabolism in vitro using a novel HP microNMR system and in vivo using our newly synthesized HP [U-²H,2-¹³C] fructose. Pharmacokinetic data for ketohexokinase (KHK) inhibition in vivo is also included to extend the blood half-life of circulating fructose in Example 3 (FIG. 11) and tumor fructose in Example 4 (FIG. 12).

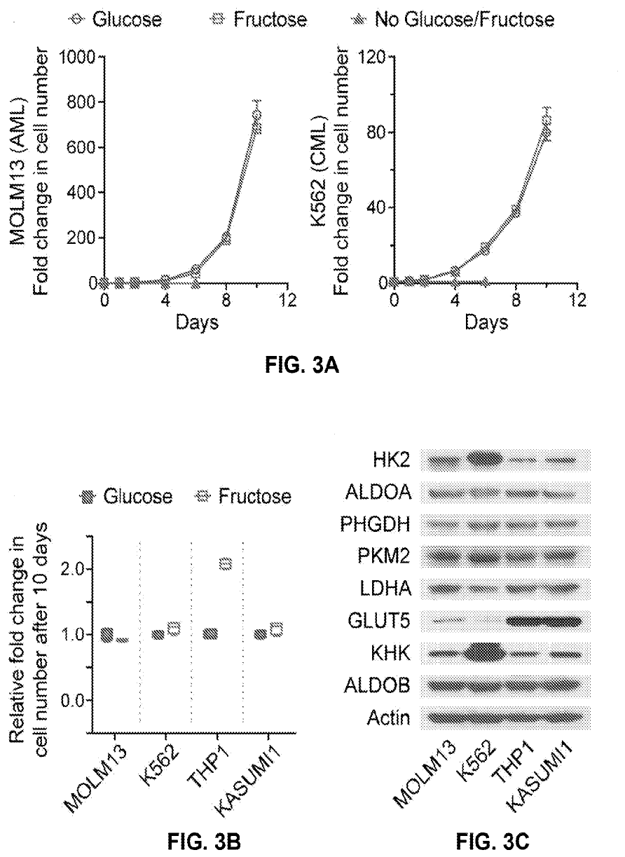

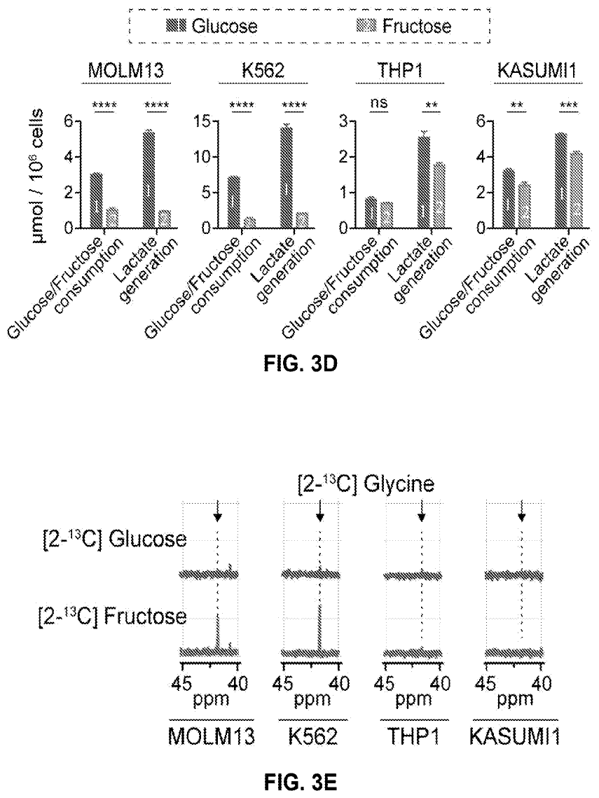

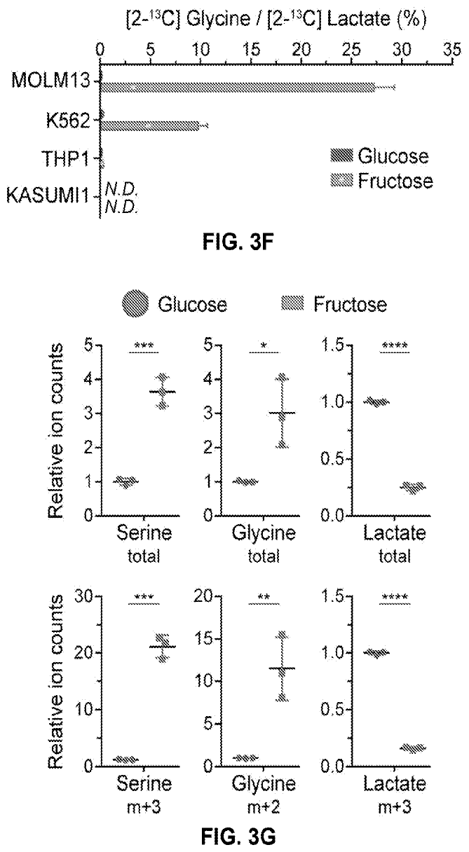

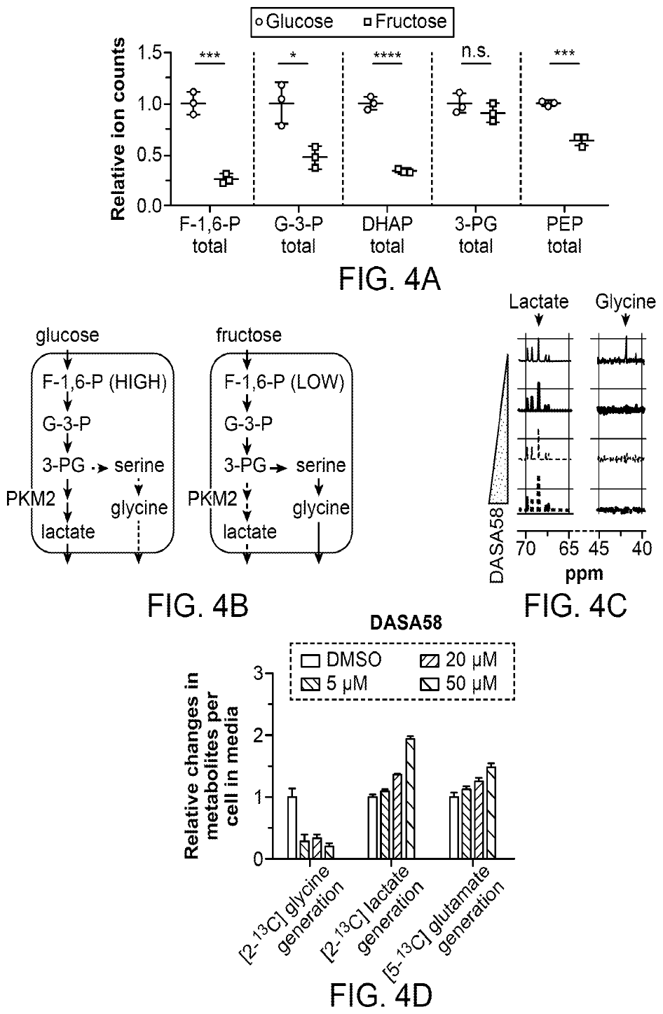

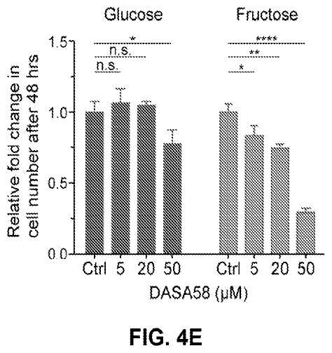

Fructose metabolism in the setting of low GLUT5 expression is shunted and can be rescued by GLUT5 overexpression: leukemia cell lines were utilized as models for differential fructose uptake. When cultured in media with 10 mM glucose or fructose, the growth rates were similar or higher in the presence of fructose (FIG. 3). These cell lines can be divided into 2 groups, those which produced equivalent levels of lactate in the glucose and fructose conditions and those which produced less lactate in fructose. Using isotope tracing with [2-¹³C]glucose and [2-¹³C] fructose, it was found that the level of [2-¹³C]glycine was significantly higher when GLUT5-low cells were cultured in media with [2-¹³C] fructose as compared to [2-¹³C]glucose (FIG. 3), demonstrating that segregation of flux from fructose is dominated by GLUT5. Taking advantage of this label scheme provides a means of directly elucidating the routing of flux through glycolysis. The significant increase in the total and fully-enriched pool sizes of metabolites indicated that the flux through SSP was elevated in GLUT5-low leukemic cells cultured in the fructose-rich condition. It was further confirmed that this was due to the inhibition of a downstream glycolytic enzyme (PKM2) as a result of low levels of the intermediate fructose-1,6-bisphosphate (F16P) (FIG. 4).

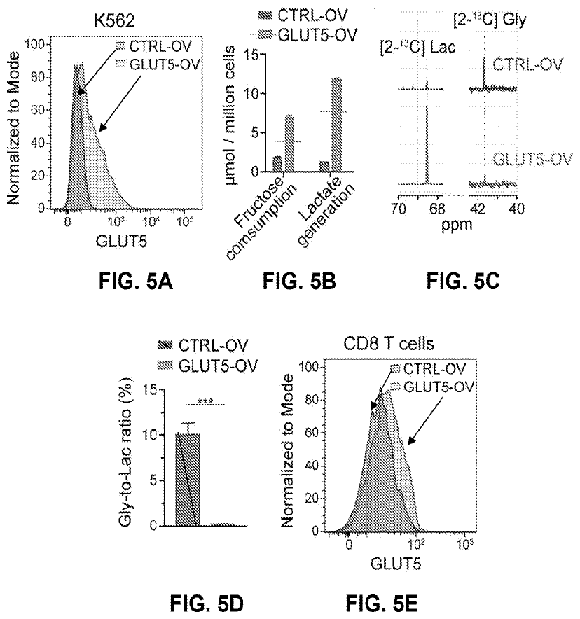

To further confirm the effect of GLUT5 level on the fate of fructose metabolism, GLUT5 was overexpressed and the metabolic phenotype was able to be reversed, metabolizing high amounts of fructose to lactate (FIG. 5). These results support the role of fructose uptake kinetics in dictating glycolytic flux and implicate its modulation as a potent mediator of glycolytic carbons.

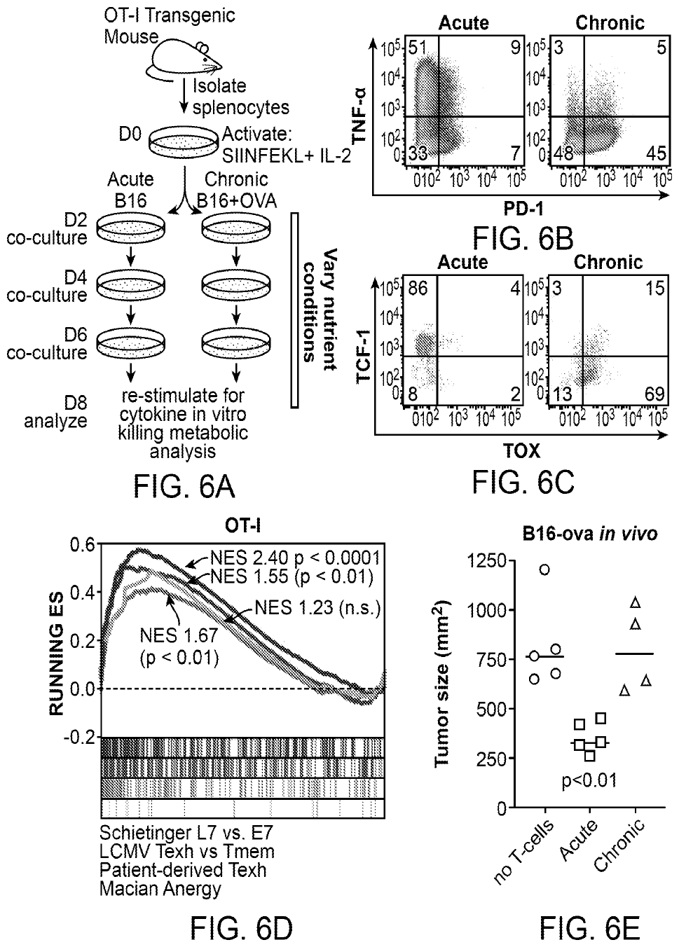

T-cell model and performance of T cells in low glucose conditions: Utilizing primary murine splenocytes, an in vitro model of T-cell exhaustion was developed (FIG. 6A). FACS analysis confirmed that chronically stimulated T-cells from the model failed to produce cytokines and upregulated immune checkpoints (FIG. 6B). Moreover, chronically stimulated T cells lost expression of memory-associated transcription factors and expressed exhaustion-associated factors (FIG. 6C). RNAseq data confirmed that the acute and chronically stimulated model matched well with multiple previously identified gene set enrichment analysis (FIG. 6D), further supporting the accuracy of this model. Taking these T cells in vivo, they also lost the ability to suppress growth (FIG. 6E).

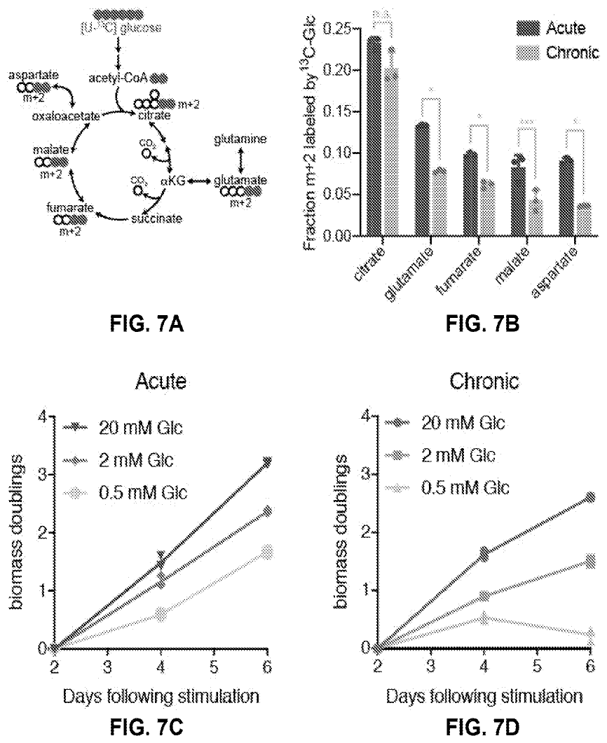

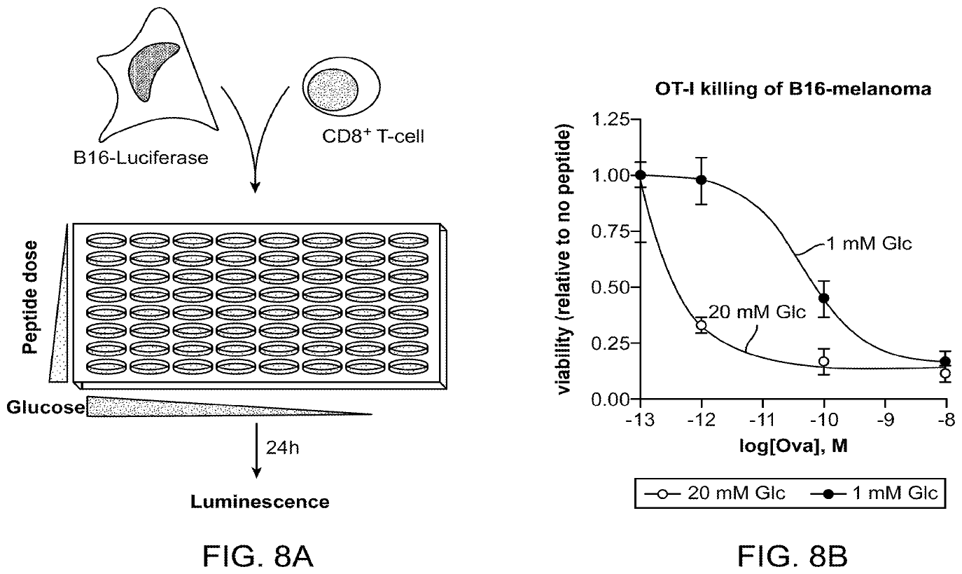

Using the system, it was found that exhausted T cells showed reduced flux of glucose into the TCA cycle (FIGS. 7A & 7B) and that under glucose-limiting conditions they demonstrated a reduced ability to double both in acute and chronic stimulation (FIG. 7C). Furthermore, when cultured in an in vitro killing assay, OT-I T cells were less able to kill B16-melanoma cells in low glucose conditions (FIG. 8).

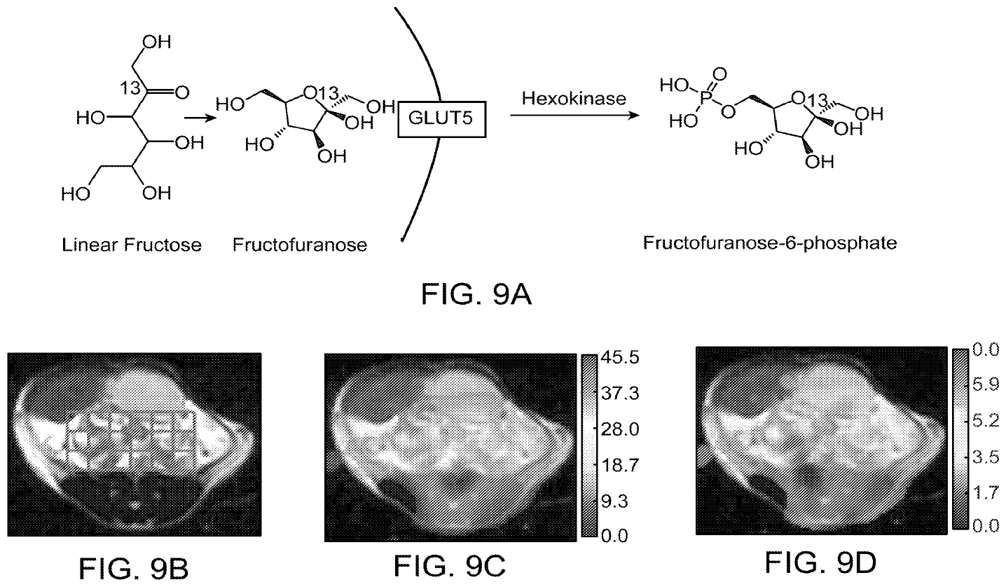

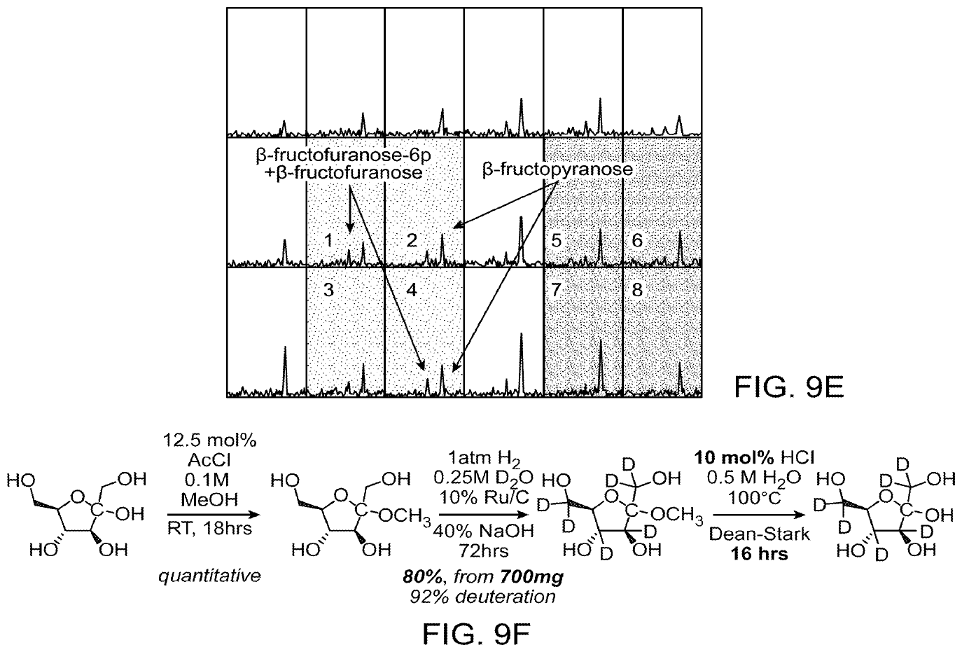

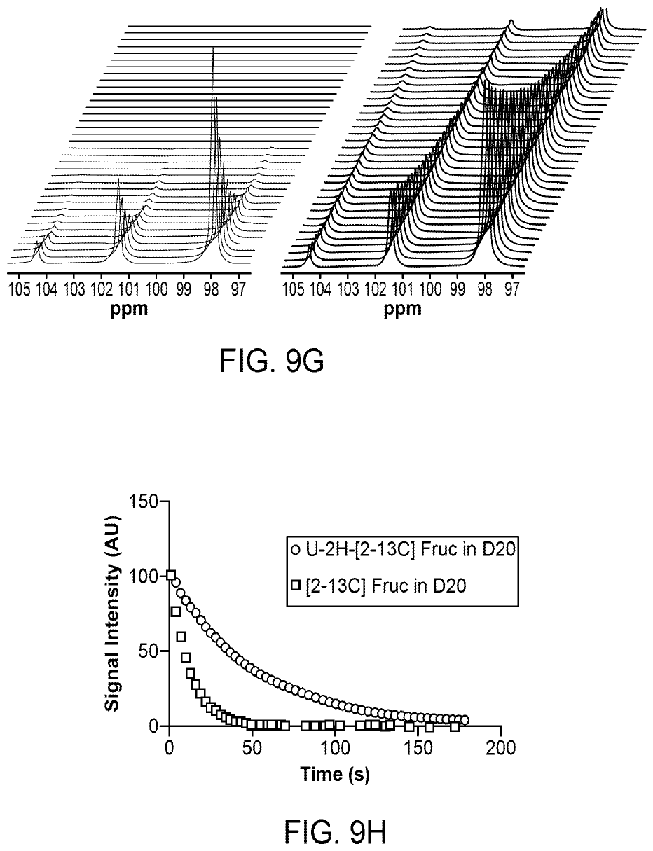

Enhancement of hyperpolarized lifetimes using D₂O solvation and application to HP fructose: In previous experiments, the necessary strategies and combined methods to develop not only novel probes for HP MRI but also bolus tracing experiments to confirm biochemistry in vivo. See Salamanca-Cardona et al. Cell Metab 26, 830-841 e833 (2017). While many molecular strategies have been developed for HP MRI, they generally suffer from short lifetimes (≤10 s) making flux through pathways of interest difficult to visualize in vivo. It was demonstrated herein that the first HP sugar probe, HP [2-¹³C] fructose, showed that it has the potential to trace in vivo metabolism even with a short lifetime of 13 s, and visualized its conversion to F6P (FIG. 9). See Keshari et al. J Am Chem Soc 131, 17591-17596 (2009). In recent work, It was shown by the Applicant that utilization of rapid exchange between H₂O and D₂O can dramatically increase the lifetimes of the HP probes by greatly reducing dipolar relaxation. See Cho et al. J Magn Reson 295, 57-62 (2018). As such, a synthesis was developed for [2-¹³C,U-²H₇] fructose at gram scale (FIG. 9F) as well as a preparation to hyperpolarize it. It was found by the Applicant that the lifetime of this molecule is 1.5 minutes, 6 times longer than previously shown (FIGS. 9G & 9H), and this can marry well with the T-cell studies.

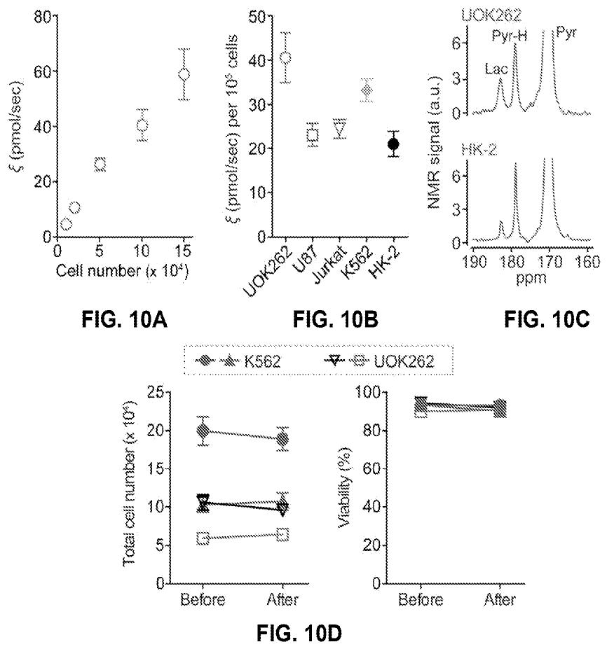

Hyperpolarized microNMR for measuring flux in small cell numbers: Given that the number of T cells engineer is limited, tools to aid in flux analysis are needed. In previous work, developed was a microNMR system capable of analyzing flux in mass limited samples (FIG. 10). See Jeong et al. Sci Adv 3, e1700341 (2017). Using it, metabolic flus were able to be measured in primary stem cells isolated from mice. This platform is used in combination with HP fructose and isotope tracing in order to assess changes in T-cell metabolism.

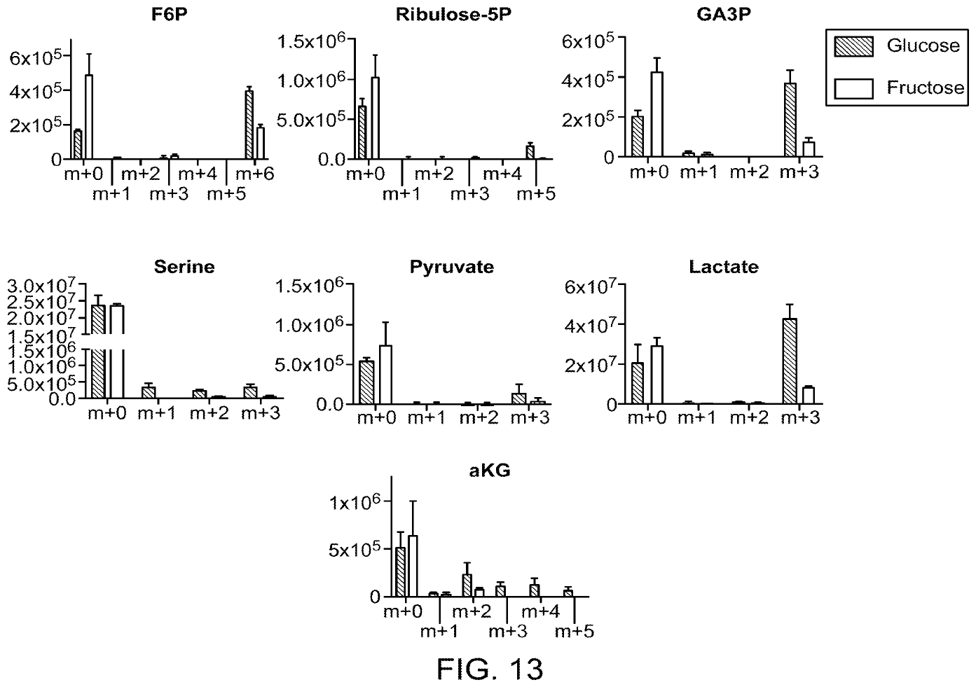

Under one experimental setting, antigen stimulated primary human T cells were incubated with either [U-¹³C]glucose or [U-¹³C] fructose for 24 hours. Activated T cells did not populate glycolytic intermediates when exposed to fructose as the only carbon source, as demonstrated by reduced labeling of m+6 fructose-6-phosphate (F6P), m+3 glyceroaldehyde-3-phosphate (GA3P), m+3 pyruvate and m+3 lactate. Furthermore, activated T cells were unable to run the pentose phosphate shunt, as demonstrated by loss of labeling in ribulose-5-phosphate (ribulose-5P), as well as the serine synthesis pathway (m+3 serine). This carried into the TCA cycle, which was significantly reduced, most clearly observed in the loss of m+2 a-ketoglutarate (αKG) when T cells were provided with fructose as the only carbon source. See, FIG. 13. These data supports that primary human T cells are unable to metabolize fructose at the levels of glucose to drive glycolysis.

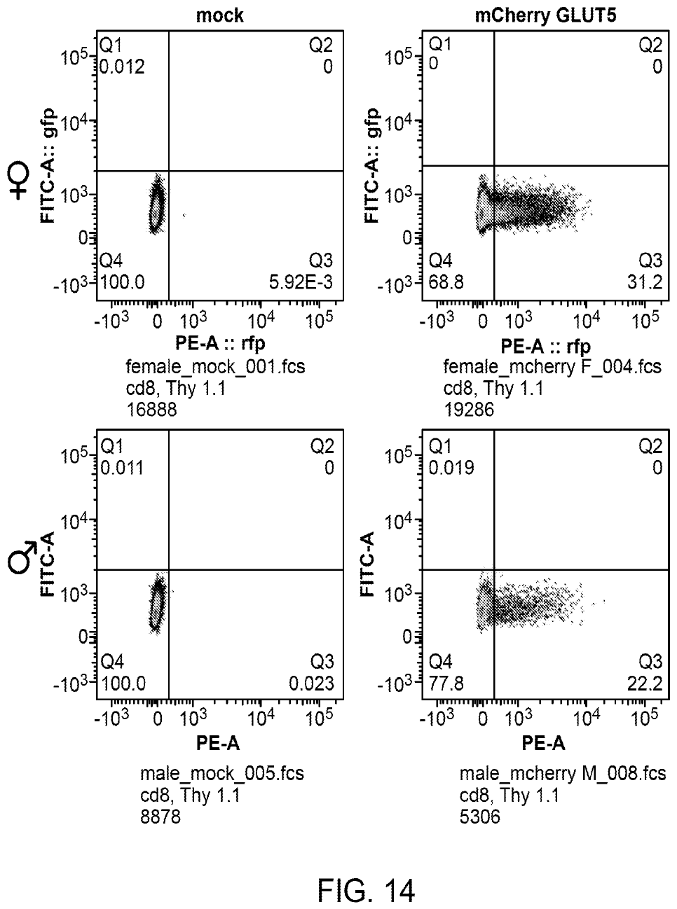

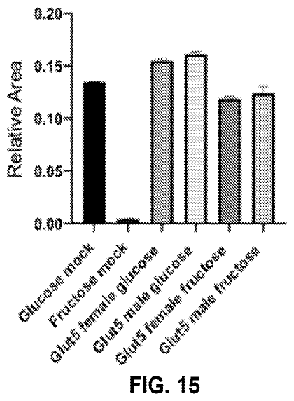

Under another experimental setting, OT-1 mice were used. These mice contain transgenic inserts for mouse Tcra-V2 and Tcrb-V5 genes. The transgenic T cell receptor was designed to recognize ovalbumin peptide residues 257-264 (OVA₂₅₇₋₂₆₄) in the context of H2Kb (CD8 co-receptor interaction with MHC class I). This results in MHC class I-restricted, ovalbumin-specific, CD8+ T cells (OT-I cells). That is, the CD8 T cells of this mouse primarily recognize OVA₂₅₇₋₂₆₄ when presented by the MHC I molecule. T-cells of the male or female OT-1 mice were engineered to (over)express GLUT5 (see SEQ ID NO: 1). Such (over)expression of GLUT5 was confirmed using flow cytometry (FIG. 14). The GLUT5-(over)expressing T-cells were then incubated with [2-¹³C]glucose or [2-¹³C] fructose for 8 hours, and ¹³C lactate derived from [2-¹³C]glucose or [2-¹³C] fructose was measured. The result is shown in FIG. 15, suggesting that (over)expression of SLC2A5 (GLUT5) in OT-1 T cells allows them to take advantage of fructose as the sole carbon source for glycolysis.

Macrophages were also investigated in addition to T-cells. The results show that primary macrophages bearing GLUT5 survive and differentiate on fructose.

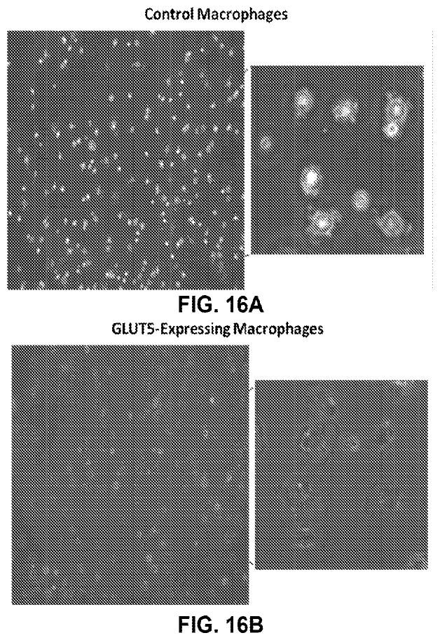

In one experimental setting, primary macrophages were cultured in media containing fructose (in the absence of glucose) for 24 hours. It was found that immune cells, including macrophages that populate the tumor microenvironment, did not express GLUT5. Corresponding to this, primary macrophages were unable to use fructose as an energy source and began to undergo de-differentiation and cell death after 24 hours of treatment (FIG. 16A). On the other hand, primary macrophages stably (over)expressing a novel GLUT5 construct (FIG. 16B) survived, differentiated, and proliferated on fructose even in the absence of glucose.

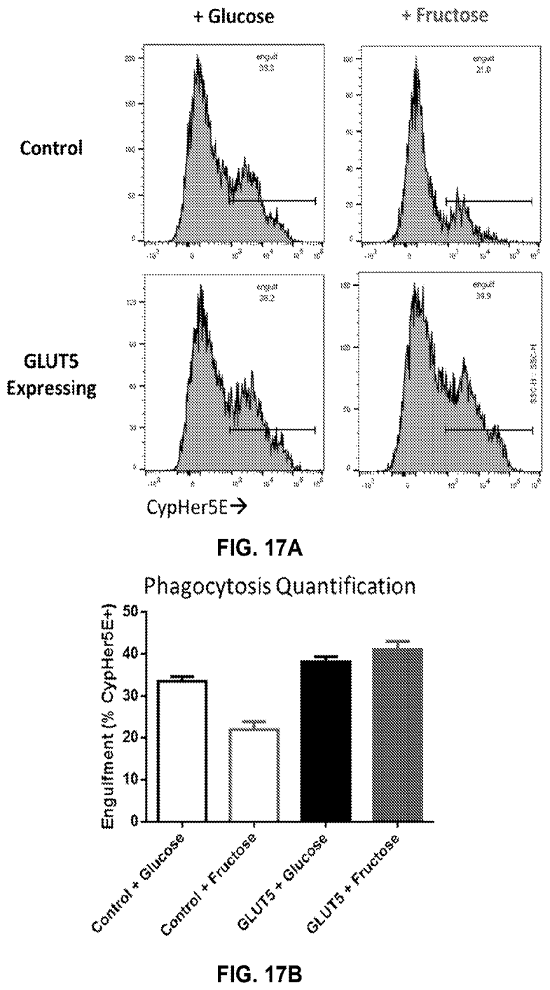

Under another experimental setting, macrophages were differentiated in glucose. MDA-MB-231 triple negative breast cancer cells (TNBCs) were labeled with the pH-sensitive dye CypHer5E, an indicator of phagocytosis, then co-cultured with macrophages (over)expressing GLUT5 or not in either glucose or fructose alone. The result is plotted in FIG. 17, showing GLUT5-expressing macrophages use fructose as efficiently as glucose to perform phagocytosis (see, the right bar of FIG. 17B). Thus, the data suggests expression of GLUT5 in macrophages allows for fructose use during phagocytosis of TNBCs.

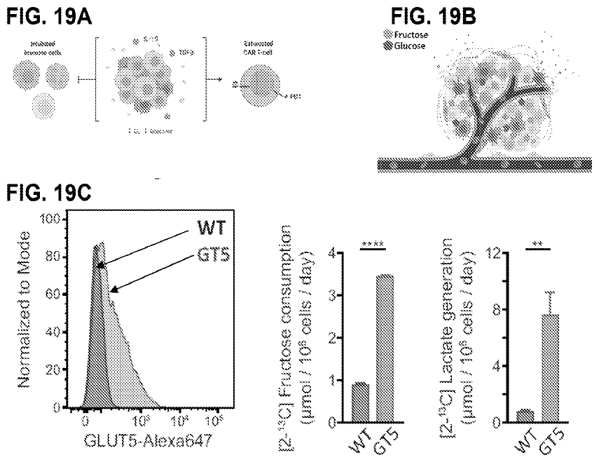

FIG. 19A shows a schematic of the immunosuppressive tumor microenvironment (TME). FIG. 19B shows a schematic of how glucose is limiting in the (TME) which makes T cells exhausted; fructose is present in the TME and is not generally consumed by cancer cells or immune cells. FIG. 19C shows that expression of fructose transporter GLUT5 (GT5) on T cells allowed them to consume fructose and metabolize it, producing lactate.

### Example 2: Trace the Metabolic Fate of Fructose in Activated T Cells to Optimize Conditions for GLUT5 Overexpression Using LC/MS Metabolomics and a Novel Model of T-Cell Exhaustion In Vitro

Utilizing a combination of nuclear magnetic resonance (NMR) and liquid chromatography-mass spectrometry (LC/MS) approaches, metabolism is traced in a novel model of T-cell exhaustion. Moreover, GLUT5 is (over)expressed in T cells and their ability to target cancer cells as mediated by enhanced glycolytic flux is characterized. Further, GLUT5 (over)expression is combined with checkpoint inhibition.

Data, such as those discussed in Example 1, demonstrates established models and methods necessary to explore fructose metabolism in the setting of T-cell activation. Moreover, modulation of GLUT5 expression can shift carbons into glycolysis to generate lactate. Accordingly, experiments are performed to trace fructose metabolism in activated T cells as well as to develop GLUT5 (over)expressing T cells and assess the ability to reroute metabolism in glucose-limiting conditions in vitro.

Trace Metabolism Using a Combination of NMR and LC/MS Approaches in T Cells with Exhaustion in Culture in Order to Determine the Metabolic Fate of Fructose

In order to understand how T cells metabolize fructose in acutely and chronically activated states, the consumption rate is firstly quantified of metabolites, including fructose, glutamine, and other substrates, by measuring changes in metabolite levels in cell culture media with high-field NMR. The pool size of intracellular metabolites and fractional labeling are also analyzed using high-field NMR and LC/MS. Together these provide a quantitative description of the pathways that utilize fructose.

In vitro T-cell exhaustion model (FIG. 6): Splenocytes from OT-I TCR transgenic mice are incubated with 1 μM SIINFEKL (SEQ ID NO: 40) peptide in RPMI media containing FBS, penicillin/streptomycin, L-glutamine, 2-mercaptoethanol, and recombinant IL-2 (10 ng/mL) for 48 hours. They are then co-cultured with B16 melanoma cells that were either unpulsed (“Acute”) or pulsed with 1 μM SIINFEKL (SEQ ID NO: 40) peptide (“Chronic”). Every 48 hours, cells are similarly passaged into fresh media.

Steady state TCA cycle metabolite measurements: Metabolites are extracted with 1 mL ice-cold 80% methanol containing 2 μM deuterated 2-hydroxyglutarate (D-2-hydroxyglutaric-2,3,3,4,4-d₅ acid, d5-2HG) as an internal standard. After overnight incubation at −80° C., lysates are harvested and centrifuged at 21,000 g for 20 minutes to remove protein. Extracts are then dried in an evaporator (Genevac EZ-2 Elite) and resuspended by incubating at 30° C. for 2 hours in 50 μL of 40 mg/mL methoxyamine hydrochloride in pyridine. Metabolites are further derivatized by addition of 80 μL of MSTFA+1% TCMS (Thermo Scientific) and 70 μl ethyl acetate (Sigma) and then incubated at 37° C. for 30 minutes. Samples are then analyzed using an Agilent 7890A Gas Chromatograph (GC) coupled to Agilent 5977C mass selective detector. The GC will be operated in splitless mode with constant helium gas flow at 1 mL/min. 1 μl of derivatized metabolites are injected onto an HP-5MS column and the GC oven temperature ramped from 60° C. to 290° C. over 25 minutes. Peaks representing compounds of interest are extracted and integrated using MassHunter software (Agilent Technologies) and then normalized to both the internal standard (d5-2HG) peak area and protein content of duplicate samples as determined by BCA protein assay (Thermo Scientific).

Isotope tracing studies: For isotope tracing studies, cells are washed and cultured in RPMI media lacking glucose or glutamine, supplemented with dialized FBS, penicillin-streptomycin, 2-mercaptoethanol, and recombinant IL-2 (10 ng/mL), as well as either ¹²C-glucose (Sigma) and ¹²C-glutamine (Gibco) or the ¹³C versions of each metabolite, [U-¹³C]glucose, [U-¹³C]glutamine or [U-¹³C] fructose (Cambridge Isotope Labs) to a final concentration of 10 mM (glucose) and 2 mM (glutamine) analogous to the data shown in Example 1 (FIG. 7). Enrichment of ¹³C is assessed by quantifying the abundance of the following ions: aKG, m/z 304-318; aspartate, m/z 334-346; citrate, m/z 465-482; fumarate, m/z 245-254; glutamate, m/z 363-377 and malate, m/z 335-347. Correction for natural isotope abundance is performed using IsoCor software. For measurements of labeled lactate and glycine, ¹³C NMR spectroscopy is used to quantify the generation of peaks in time and processed using a combination of mestReNova (mestrelab) and Chenomx (Chenomx, Inc) software packages.

### Overexpress GLUT5 in T Cells and Demonstrate Enhanced Ability to Kill B16 Melanoma Cells as Result of Enhanced Glycolysis Mediated by Fructose

The changes in fructose metabolism in T cells are determined with GLUT5 overexpression (GLUT5-OV). The data with leukemia cell lines show that GLUT5-OV allows the cells to metabolize fructose through the glycolytic pathway at a similar rate as glucose. Retroviral particles for GLUT5-OV are utilized in T cells and the assay is optimized to maximize their fructose metabolism supported by Example 1 (FIG. 5E). With GLUT5-OV, the expression level of interferon-gamma (IFN-7), a molecular signature for the effector T cells, is evaluated, along with proliferation rate of T cells in the chronically-activated state. Cell killing is measured as in Example 1 (FIG. 8) by co-culturing lucerifase-expressing B16 cells incubated at varying levels of glucose and fructose and pulsed with SIINFEKL (SEQ ID NO: 40) peptide. Further, the advantage of incubating cells with checkpoint blockade (anti-PD-L1) is explored in vitro with GLUT5-OV and extracellular fructose in the presence of B16 cells.

Biostatistics, analysis and sample: LC/MS and NMR metabolomics are processed using previously described methods and stable isotope tracing is quantified as described. All metabolic levels are compared between the different conditions (combinations of activation state and GLUT5 status, 4 groups) using the t-test. With 10 in vitro replicates per group, there is an 80% power at a two-sided 0.05 level to detect Cohen's d effect size of 1.3. The Bonferroni correction is used to account for multiple testing.

Results, potential challenges and alternative solutions: Without wishing to be bound by the theory, with GLUT5 in T cells derived from OT-I TCR transgenic mice, an increased steady state concentration of lactate can be detected as well as glutamate and other TCA cycle intermediates in both the acute and chronically stimulated T cells and that this is derived from ¹³C-fructose. Under the conditions of enhanced fructose uptake and low glucose (0.5-1 mM), T cells can secrete levels of IFN-γ at an equivalent or higher level as the high glucose condition (20 mM). Moreover, the mass doubling rate can be restored to the high glucose condition. Furthermore, these enhanced T cells can have the ability to more efficiently kill B16 melanoma cells in culture.

It is possible that the endogenous levels of the fructose transporters GLUT2/5 provide some level of fructose transport into the melanoma cells to allow them to compete with T cells in fructose-rich conditions. Without expected to be limiting, given that fructose transport is disproportionally increased in T cells; however, the titration of transport is explored in order to optimize a balance between metabolic flux, killing and competition with the target cancer cell. It is also possible that glucose may not be limiting in the tumor for T cells; while this is unlikely (Sugiura & Rathmell. J Immunol 200, 400-407 (2018)), the ability of GLUT5 overexpression to enhance metabolism even in the setting of physiologic glucose (5 mM) is explored. With co-incubation with anti-PD-L1 antibody, further killing efficacy can be observed in GLUT5 OV T cells. In some embodiments, these cells express fewer exhaustion (e.g., Tox) (Philip et al. Nature 545, 452-456 (2017); Scott et al. Nature 571, 270-274 (2019)) markers and, at a lower glucose concentration, show a greater ability to kill cells than wildtype T cells with checkpoint blockade. Ultimately, evidence is provided showing that such a nutrient strategy could “turbo charge” T cells.

### Example 3: Develop a Non-Invasive Imaging Approach to Trace Increased Flux from Fructose

New technologies are needed to advance the understanding of T-cell metabolism. Using novel methods to interrogate metabolism in vivo, hyperpolarized [U-²H,2-¹³C] fructose is developed for measuring flux in glycolysis and is utilized it in the setting of a novel platform, hyperpolarized microNMR, and in vivo in animal models.

Many mechanisms have been developed to assess and modulate metabolism in vitro, providing a basis for understanding of immunometabolism. However, the difficulty of applying these approaches in vivo limits the ability to characterize in vivo metabolism and the results of immune cell modulation. Accordingly, the disclosure herein is to develop a unique strategy combining in vivo isotope tracing of isotopically enriched fructose with hyperpolarization to provide a means of tracing metabolism in vivo.

Optimize Hyperpolarized [U-²1,2-³C] Fructose for Measuring Flux into Glycolysis

While it has been previously shown that fructose can be hyperpolarized, it suffers from a short T₁, limiting its ability to robustly detect metabolic flux through glycolysis all the way to lactate. To address this, a novel synthetic strategy was developed to uniformly deuterate and carbon label the C2 hemiketal position of fructose as well as dramatically extend its lifetime to 1.5 mins (Example 1, FIGS. 9G & 9H). The currently achieved polarization is greater than 10%—superior to most hyperpolarized probes currently in development. In order to make this new approach robust for in vitro and in vivo studies, the concentrations of polarizing radical (OX063, typically 15-30 mM) and substrate are optimized in the preparation (currently 4M), with achieved concentration at dissolution and maximum hyperpolarization as the metrics. The synthesis is scaled up and grams of [U-²H,2-¹³C] fructose is produced for use in subsequent experiments. Given Applicant's extensive previous experience developing molecules for hyperpolarized MRI, greater than 20% polarization and 100 mM HP [U-²H,2-¹³C] Fructose (TIP fructose) in solution can be produced for use in both in vitro and in vivo studies.

Utilize a Novel microNMR System to Measure Flux in T Cells, Validated by Isotopic Tracing

Utilizing the newly developed HP fructose preparation, the flux of fructose into glycolysis is traced in T cells. Fructose is hyperpolarized as optimized in Examples and dissolved at concentrations ranging from 10-100 mM for cell studies. GLUT5-OV K562 cells (Example 1, FIG. 5) are used as a model system to optimize the data acquisition and read out of HP fructose tracing in vitro (n=10 replicates). The newly developed HP microNMR (Example 1, FIG. 10) is used to measure metabolism in these cells as previously described. See Jeong et al. Sci Adv 3, e1700341 (2017). Briefly, the system operates at 3T (32 Mhz for ¹³C) and hyperpolarized spins are excited using a 30 degree flip angle and acquired with a TR=250 ms. The spectral data is processed using standard methods (Fourier transform, 5 Hz line broadening, baseline correction and peak integration). It is firstly measured to what degree HP fructose is converted to HP F6P in vitro and assess if HP C2 lactate is readily detected. C2 lactate (resonating at 70 ppm) has been detected previously by HP fructose in E. coli and HP glucose and HP pyruvate in mammalian cells (Meier et al. Mol Biosyst 7, 2834-2836 (2011); Meier et al. FEBS Lett 585, 3133-3138 (2011); Timm et al. Magn Reson Med 74, 1543-1547 (2015); Rodrigues et al. Nat Med 20, 93-97 (2014)). These products are used to optimize the acquisition strategy and determine an adequate number of cells for the T-cell experiments. In additional parallel studies, [U-¹³C] fructose is incubated with cells at increasing time points (0, 1 hr, 4 hr, 8 hr, 24 hr) to measure metabolic flux for quantitative comparison to HP measurements. Cells are extracted and metabolites are measured using LC/MS as described herein. Then, using the T-cell models described in Example 2 (acute, chronic and GLUT5-OV, n=10 cell replicates per group), the metabolic flux of HP fructose to its products is measured and changes in T-cell metabolism are assessed in the HP microNMR system. In addition, HP pyruvate NMR and [U-¹³C]glucose are utilized with LC/MS to measure glycolytic flux for comparison to fructose measurements.

### Develop and Implement In Vivo HP Fructose MRI

Translation of novel hyperpolarized probes into in vivo animal models represents a great technical advance in the exploration of the modulate metabolism induced by GLUT5-OV. Using the optimized probe formulation for HP fructose, the metabolic conversion of fructose is explored in vivo in the liver of wild type B6 mice, as previously shown. In some embodiments, fructose is converted to fructose-1-phosphate (F1P) and later to lactate in vivo and that this can be readily blocked by inhibition of ketohexokinase (KHK). [2-¹³C]F1P resonates downfield of the C2 pyranose resonance; in Example 1, this conversion was measured in transformed hepatocytes (HepG2 cells, FIG. 11) using [2-¹³C] fructose. When further converted, C2 lactate is generated (resonating at 70 ppm) from fructose and this has been detected previously in vivo derived from HP glucose (Rodrigues et al. Nat Med 20, 93-97 (2014)) and pyruvate (Park et al. Magn Reson Med 75, 973-984 (2016)). All imaging studies are conducted under IACUC-approved protocol #13-12-019. Briefly, mice are imaged using a 3T Bruker MRI equipped with a dual tune ¹H/¹³C volume coil. To measure delivery to the liver and timing of conversion, a slab dynamic approach is utilized with a constant 10° excitation and temporal resolution of 5 s, similar to previous work using HP [1-¹³C]pyruvate, after infusion of 200 μL of 100 mM HP fructose, in a subset of mice (n=5). This established timing is used to time the HP MR imaging, acquiring spatially resolved MRSI data, analogous to Applicant's previous work, after infusion of HP fructose. Mice with vehicle and treated (10 mice per group, 20 mice total) with a single dose of the KHK inhibitor 6-(4-(2-Hydroxyethyl)piperazin-1-yl)-2-(3-(hydroxymethyl)-piperidin-1-yl)-4-(trifluoromethyl)nicotinonitrile (KHKi) at 10 mg/kg are imaged as previously described. See Huard et al. J Med Chem 60, 7835-7849 (2017). Pharmacokinetic analysis of the KHKi was conducted in vivo, finding that the drug has a ti/2 of 2 hr and Cₘₐₓ of 475 μg/ml (approximately 1 μM). This is well within what Applicant found for inhibition of KHK and the sensitivity for detecting it with ¹³C-F1P in vitro (FIG. 11C). In order to further confirm the observed change in metabolism, a rapid bolus approach is used in vivo to infuse 100 mM [U-¹³C] fructose into mice and a rapid freeze clamping of the liver tissue is conducted 60 seconds post-infusion. This is then extracted and labeled metabolites are measured by LC/MS and NMR. This approach to confirm glutamine metabolism in vivo is used here for comparison to HP fructose. See Salamanca-Cardona et al. Cell Metab 26, 830-841 e833 (2017).

Biostatistics, analysis and sample: LC/MS data is processed using previously described methods and stable isotope tracing is quantified for comparison to HP data. To analyze HP fructose data, areas under the curve for all metabolites are integrated and fit to a flux model as previously described to generate rates. See Keshari et al. Magnetic resonance in medicine 63, 322-329 (2010). These is then compared in the setting of GLUT5 overexpression in order to quantify flux changes. Conversion of HP fructose to F1P is quantified by integrating the area under the curve for each voxel of the 2D spatially resolved data. The ratio of F1P/fructose is used to normalize the data. These data are then compared in the setting of KHKi in order to quantify flux changes in vivo. These data are compared to bolus infusions and LC/MS measurement of ¹³C labeled fructose and F1P in liver tissue. All metabolic changes between the different conditions are compared using the t-test. With at least 5 replicates per group, there is an 80% power at a two-sided 0.05 level to detect large Cohen's d effect size of 2. The Bonferroni correction is used to account for multiple testing. Without wishing to be bound by the theory, this is sufficient power to detect the changes in vitro (with GLUT5 overexpression) and in vivo (with KHKi).

Results, potential challenges and alternative solutions: The newly developed fructose probe can provide evidence of in vitro flux of HP fructose to lactate, demonstrating that flux from this nutrient is modulated via overexpression of GLUT5. By utilizing the HP microNMR approach, rates can be measured in line with longer term flux measurements conducted by LC/MS. Moreover, this probe can be readily detect fructose metabolism in vivo in the liver where GLUT2 expression is high, generating substantial amounts of F1P, and that this can also be validated by rapid infusions of [U-¹³C] fructose, providing a unique approach to studying this modulation. It is possible that the doses of HP fructose can not be sufficient to detect metabolism in vivo; increased dosing up to the 250 mM dose used for HP glucose is explored in the in vivo studies. Furthermore, modulation of fructose uptake using the KHKi in vivo can be reflected in the imaging with a reduced F1P resonance. It is possible that a single dose of the KHKi may not be sufficient to reduce the flux; added doses are explored to match the measured pharmacokinetics of the drug. Overall, Developed and used in vivo herein is a novel strategy to explore fructose immunometabolism.

### Example 4. Utilize GLUT5-Overexpressing T Cells In Vivo with Addition of Fructose to Demonstrate Enhanced Tumor-Killing Efficacy and Verify Metabolism Using In Vivo Isotopic Tracing and Hyperpolarized MRI

Using a syngeneic model of melanoma, methods to raise the concentration of fructose in tumor is assessed and in turn a tumor microenvironment more amenable to GLUT5 T cells is created. T-cell trafficking to the tumor and metabolic function using in vivo rapid isotopic tracing is investigated and response to T-cell treatment is assessed as well as combination with checkpoint blockade.

Melanomas, which are known to respond to immunotherapy (see Weiss et al. Clin Cancer Res 25(17):5191-5201 (2019)), express low levels of GLUT2/5 as well as KHK (Uhlen et al. Science 357(6352):eaan2507 (2017); Cerami et al. Cancer Discov 2, 401-404 (2012); Gao et al. Sci Signal 6, p11 (2013)), making them an ideal target to demonstrate the enhanced killing ability of GLUT5-overexpressing T cells. Using a syngeneic model of melanoma, developed herein are strategies to increase tumor fructose and leverage this new nutrient availability to fuel enhanced T cells in vivo. Furthermore, the isotope tracing and HP MRI tools developed are used to interrogate metabolism in vivo and mechanistically link the changes in therapeutic response observed with the underlying immunometabolism.

### Utilizing a Syngeneic Model of Melanoma, Assess the Ability to Raise the In Vivo Fructose Level in Tumors by Exploring Multiple Fructose Delivery Routes (IV, IP and High Fructose Corn Syrup) and Ketohexokinase Inhibition

In order to determine the effect of fructose addition into GLUT5-OV T cells in vivo, the assay is optimized for delivering fructose into the tumor microenvironment. Specifically, three methods are tested, including: (i) intraperitoneal (IP) and (ii) intravenous (IV) injections of fructose, and (iii) water bottle with high-fructose corn syrup (HFCS). IP and IV injections (4 g/kg) are conducted daily and 25% HFCS in water is provided for three weeks; twice a week during this time the fructose level in circulating blood is measured and an endpoint tumor fructose measurement is taken. A B16 melanoma mouse model is utilized. Briefly, B16 cells (2×10⁵) are engrafted subcutaneously into C57BL/6 mice and assayed on Day 7 post-tumor-engraftment. In a second cohort of mice, with the same conditions, co-administration of the KHKi⁵³ used in Example 3 is performed to block the uptake of the liver and small intestine (the predominant sites of fructose uptake and subsequent phosphorylation by KHK). As shown in Example 1, a single IP injection of fructose was piloted and both the tumors and liver were extracted after 1 hr (FIG. 12). This data shows that not only there are mM concentrations of fructose in the tumor but this is further enhanced with a single dose of KHKi. Given this PK data, this dose of KHKi is explored using multiple routes of delivery. Specially, a dose of 10 mg/kg was used and administered IP at the same time as the fructose delivery. Fructose concentration is measured in the circulating blood as well as the endpoint tumor with all 3 delivery methods (n=10 mice per group, total of 80 mice) utilizing a standard kit assay. See Goncalves et al. Science 363, 1345-1349 (2019). Taken together, these experiments provide the necessary data to determine which fructose delivery method is best for the remaining experiments.

### Characterize to What Degree GLUT5-OV T Cells Traffic to the Tumor and Exhibit Enhanced Glycolytic Flux In Vivo by Applying Rapid Isotopic Tracing

The method that exhibits the highest concentration of fructose to the tumor-bearing mice with an infusion of T cells is applied. On Day 7 post-tumor-engraftment, the GLUT5-OV or Ctrl-OV T cells are transferred into the tumor-bearing mice through intravenous injection. Recently, one study showed that the basal level of fructose in circulating blood of C57BL/6 mice is 2 mM (Goncalves et al. Science 363, 1345-1349 (2019)); therefore firstly tested is whether GLUT5-OV itself can improve T-cell antitumor function in the tumor microenvironment (TME). Tumor size is measured every two days after T-cell transfer using the in-house preclinical MRI machine (n=5 per group, with a total of 6 groups—30 mice: GLUT5-OV and Ctrl-OV with ±fructose as well as just ±fructose in the absence of T-cells for comparison). Also tested is whether GLUT5-OV T cells utilize fructose for their major carbon source in another set of tumor-bearing mice; on Day 10 post-T-cell transfer, [U-¹³C] fructose is injected through tail vein, T cells are collected from tumor, sorted based on GLUT5, and their intracellular metabolites are analyzed with LC/MS.

### Assess In Vivo Tumor Response Using Both Conventional Anatomic MRI and HP MRI

This work is extended to imaging of the B16 melanoma model with treatment in order to validate in vivo target efficacy as well as the added benefit of checkpoint blockade. Post-development of tumors, mice (n=30 mice) are imaged with HP fructose at baseline in order to assess dynamics in vivo. All imaging studies are conducted under IACUC-approved protocol #13-12-019. To measure delivery to the tumor and timing of conversion, a slab dynamic approach is used with a constant 100 excitation and temporal resolution of 5 s, after infusion of 200 μL of 100 mM HP fructose, similar to previous work using HP [1-¹³C]pyruvate, in a subset of mice (n=5). Once timing is established, this is used to time the HP MR imaging, acquiring spatially resolved MRSI data analogous to previous work after infusion of HP fructose. Anatomic images are acquired on these mice using both T₁-weighted gradient echo and T₂-weighted spin echo sequences. Images are acquired before and after administration of either vehicle, Ctrl-OV or GLUT5-OV T cells at timings of baseline, 24 hours, 5 days and 2 weeks. Peak areas under the HP MRSI data are quantified for each metabolite and compared to surrounding tissue. These mice are also injected with HP pyruvate to assess the total glycolytic metabolism in the tumors in order to establish a possible re-routing of glycolytic flux in tumors. Tumor volume is calculated from the anatomic MRI and used to monitor changes in tumor size over time. After this study, in a second cohort of mice (n=20) GLUT5-OV T cells are infused and treatment with 250 μg of either anti-PD-L1 antibody or isotype control antibody is performed (treated on days 3, 5, 7 and 9 post-engraftment) (Zamarin et al. J Clin Invest 128, 1413-1428 (2018)). These mice are monitored analogous to the first cohort.

Biostatistics, analysis and sample: In some embodiments, the level of fructose increases with successful fructose administration, and with 10 replicates per group there is a power of 80% to detect a Cohen's d effect size of 1.2 with a one-sided 0.05 level. The ANOVA method is used to look for differences by method of delivery and KHK inhibition. This same comparison is made when inhibiting KHK and in some embodiments, a larger increase is shown, thus having more power to detect it. There are 5 replicates per group and the degree of ¹³C labeled products are measured after infusion of ¹³C-fructose in tumors as well as tumor size at 10 days after T-cell infusion. There is an 80% power to detect an effect size of 1.4 with at least a one-sided 0.05 level test. Conversion of HP fructose to HP lactate is quantified by integrating the area under the curve, and the ratio of HP lactate/fructose is compared for each group. In addition, HP lactate derived from HP pyruvate is acquired for comparison and quantified the same way. Tumor volumes are quantified by standard ROI analysis of tumors on T₂-weighted images and calculated for each time point. Percent change in tumor volume is used as the metric for response. With at least 5 replicates per group there is an 80% power at a one-sided 0.05 level to detect large Cohen's d effect size of 1.4. In all comparisons, Bonferroni correction is used to account for multiple testing.

Results, potential challenges and alternative solutions: In some embodiments, high dose injections of fructose allows for a transient but substantial increase in blood as well as tumor fructose levels (>2 mM). It was found by Applicant that with a single IP injection of fructose the blood concentration can be raised to >10 mM and tumor concentration can be raised to >2 mM. The dosing schedule relative to T-cell action is further optimized. It is possible that the fructose levels of tumors can not be further elevated in vivo due to the action of the liver and gluconeogenesis. Levels of glucose in the blood are measured in order to assess this effect and the addition of the KHKi further addresses this limitation, since Applicant was able to extend the blood half-life of fructose 50% in the first attempt with the KHKi. Additionally, if this dose of KHKi is not adequate, the possibility of increasing the dose is explored in order to further inhibit KHK. It is important to note that the T cells do not express significant KHK and this is not relevant for their metabolism. In some embodiments, the KHKi is necessary as the blood fructose is high after injection, but if it is necessary and if the KHKi does not dramatically increase the fructose half-life in the tumor, further explored is the development of liposomes containing fructose as well as direct injection of fructose into tumors in order to verify the fundamental metabolic reprogramming. This facilitates not only enhanced trafficking and survival of T cells in the tumor microenvironment, but also more substantial killing as further manifested in reduced tumor sizes. Moreover, in some embodiments, metabolism of HP fructose can be observed in tumors that are treated with GLUT5-OV T cells, matched to tracing findings from LC/MS and NMR experiments, as T cells can expand in the tumor to greater than 10% of cells per voxel. This is readily in the range of detection for HP MRI. It is possible that HP lactate can not be detected in tumors from fructose due to the cellularity. At the minimum, however, HP F6P can be detected, which was previously detected in tumors that overexpress GLUT5; this informs on the degree of fructose metabolism into glycolysis. In some embodiments, anti-PD-L1 co-therapy further enhances the efficacy of GLUT5-OV T cells in vivo and that this is reflected in tumor volume in time. This not only demonstrates that T cells are able to metabolize fructose in vivo, but also validates the development of this translatable biomarker. Furthermore, this provides support for future studies in combination with checkpoint blockade and other solid tumors.

### Example 5. Utilizing Fructose Metabolism to Fuel and Image Anti-Tumor Immunity

T cells were engineered to overexpress a fructose transporter, GLUT5, which is normally not expressed in the immune compartment at high levels. Without wishing to be bound by theory, it was believed that increased fructose uptake by CD8 T cells will ameliorate exhaustion caused by the glucose-low tumor microenvironment.

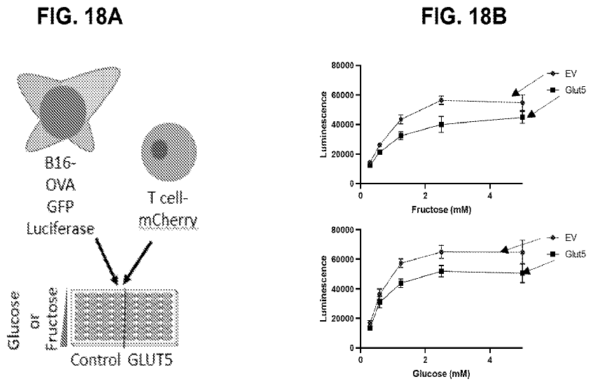

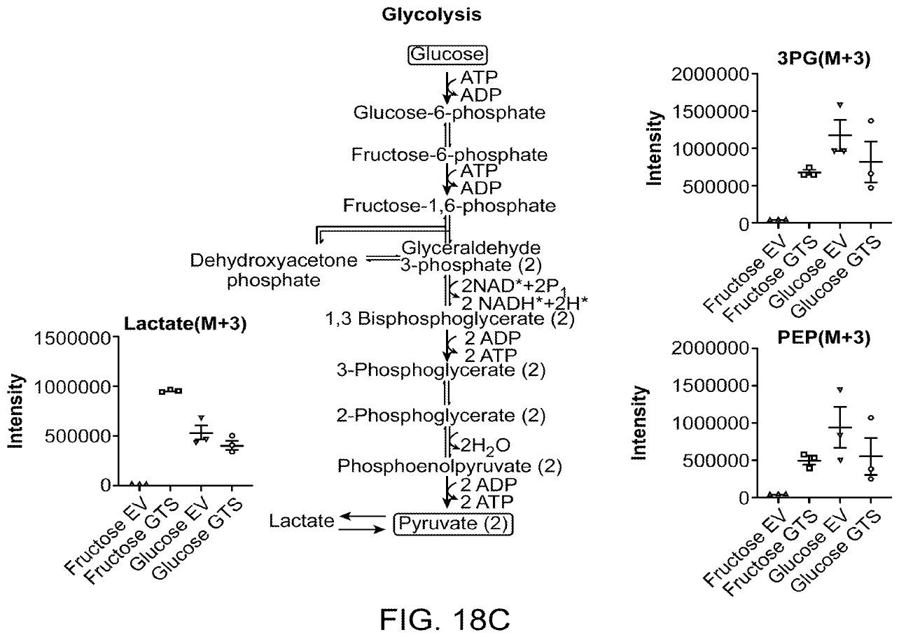

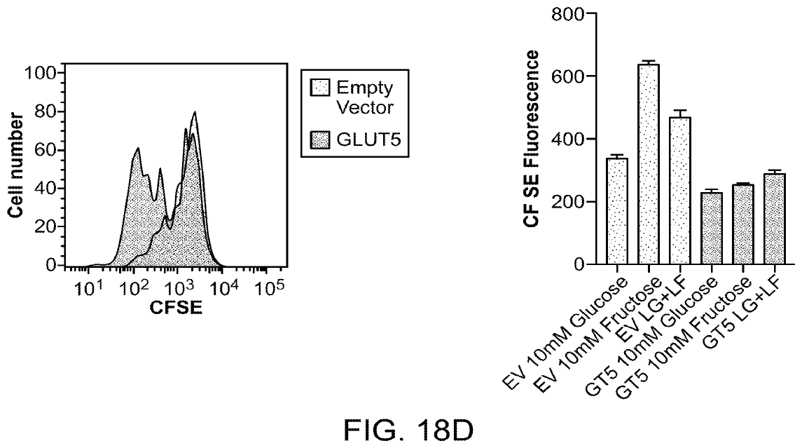

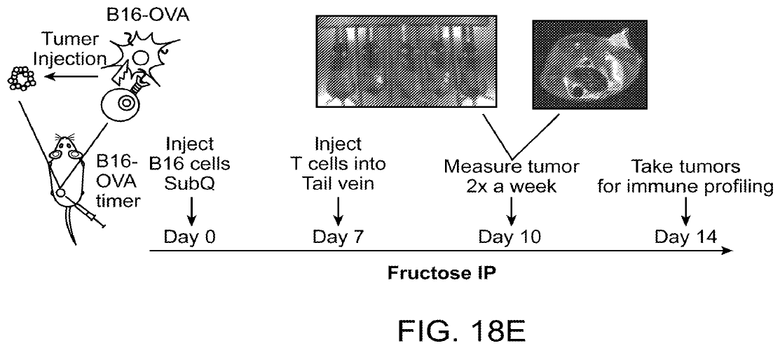

The OTI mouse model of anti-tumor immunity was utilized in which all CD8 T cells were engineered to recognize OVA peptide presented on MHCI. For the tumor model, B16 melanoma cells overexpressing OVA in conjunction with GFP and Luciferase were chosen for in vivo and in vitro imaging. An assay was then designed to measure cancer cell killing in vitro using T cells which express GLUT5 coupled to mCherry and B16-OVA-GFP-Luciferase cells (FIG. 18A). Live B16 cells were quantified post incubation with T cells under various glucose and fructose titrations using luminescence (FIG. 18B). GLUT5 T cells were more efficient in killing under low glucose and fructose conditions, in the range of 1.5-3 mM (FIG. 18B). GLUT5 expression restored T cell glycolytic capacity under fructose to that of unexhausted T cells under glucose replete conditions (FIG. 18C). Similarly, GLUT5 expressing T cells proliferated more under high fructose and low glucose conditions, as was assayed through CFSE fluorescent dye incorporation and dilution over three-day period (FIG. 18D).

A mouse experiment to measure GLUT5 T cell tumor killing in vivo is being conducted (FIG. 18E). The tumor size is quantified using luminescence and T2-weighted MRI imaging.

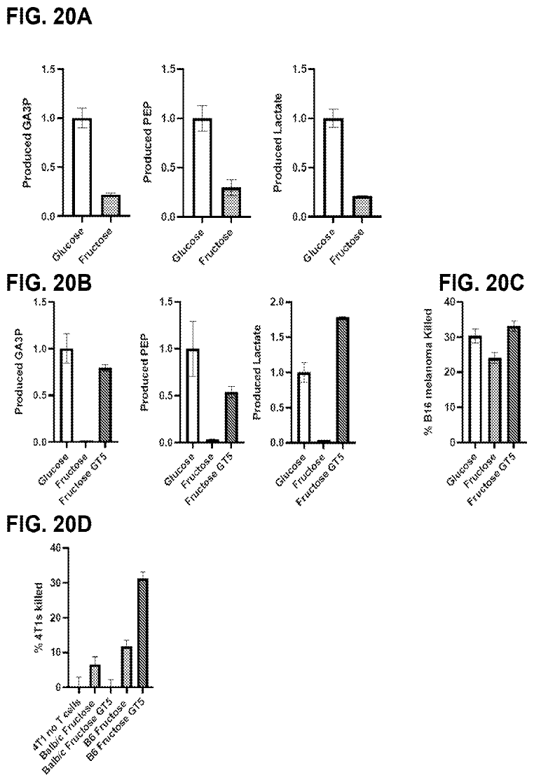

FIG. 20A shows that wild-type (WT) T cells do not metabolize fructose as evident through decreased production of glycolytic intermediates, GA3P, PEP and lactate when grown in fructose compared to glucose. FIG. 20B shows that GT5-expressing T cells grown in fructose are able to produce GA3P, PEP and lactate, which is comparable to WT T cells grown in high glucose media. FIG. 20C: GT5 or WT T cells were grown with B16 melanoma cells in glucose or fructose, and their ability to kill was evaluated. FIG. 20D: T cells were either isolated from B6 mice or Balb/c mice, engineered to express GT5, and grown with 4T1 breast cancer cells which come from Balb/c background. The ability of B6 GT5 T cells to kill based on MHC mismatch was evaluated in glucose and fructose media.

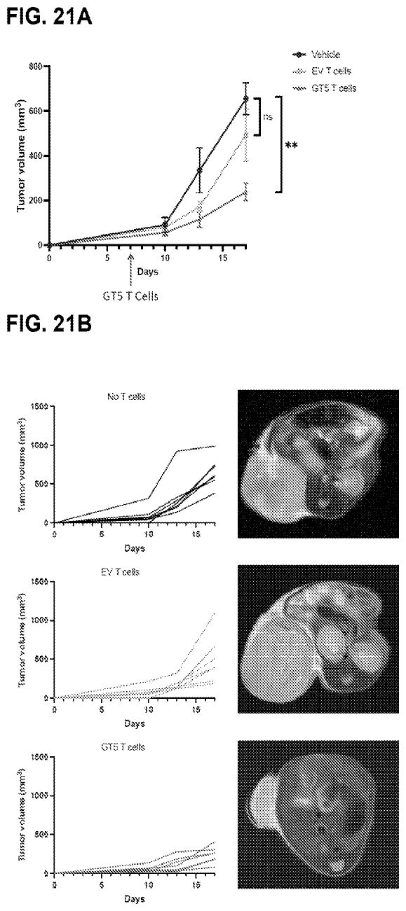

FIG. 21: The ability of GT5 cells to kill in vivo was evaluated: B16 melanoma cells were injected into mouse flanks and either WT (EV) or GT5 T cells were injected one week post tumor implantation. Figures on the right show the progression of individual mice and their representative MRI images.

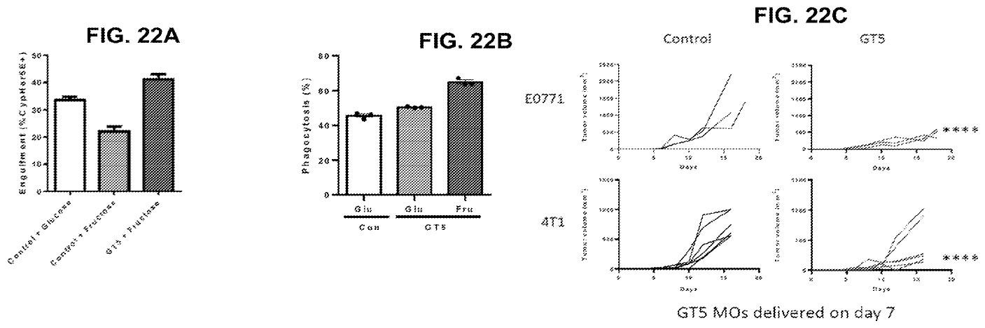

FIG. 22A: GT5 was introduced into macrophages and subsequently cultured in fructose or glucose prior to co-culture with apoptotic human breast cancer cells. Control cells (cells not given GT5) were generally unable to use fructose to perform phagocytosis of apoptotic breast cancer cells (termed efferocytosis; compare orange bars to white bars). On the other hand, macrophages with GT5 not only were able to efficiently perform efferocytosis of apoptotic breast cancer cells, they did so better than any condition tested (compare red bar to white and orange bars). FIG. 22B: Similar to FIG. 22A, except instead live human breast cancer cells were concurrently labeled with the antibody CD47, allowing macrophages to perform antibody-mediated phagocytosis. Importantly, macrophages with GT5 exhibited significantly enhanced antibody-mediated phagocytosis of CD47-labeled human breast cancer cells than all other conditions tested (compared red bar to white and orange bars). FIG. 22C: The ability of GT5 macrophages to prevent progression of two orthotopic mouse models of human breast cancer. All mice were treated with CD47 antibody beginning day 7 after implantation. Additionally, tumor-size matched mice were administered either macrophages with a control construct or GT5. Both immune checkpoint blockade (ICB)-sensitive (E0771) and -insensitive (4T1) orthotopic models significantly responded to GT5 macrophage infusion without the need for ICB therapy.

These results demonstrate that overexpressing GLUT5 on T cells are useful in methods for restoring T cell functionality in vitro and in vivo under limiting glucose conditions.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. An engineered immune cell comprising a non-endogenous expression vector that includes a nucleic acid sequence encoding a Glucose Transporter 5 (GLUT5) amino acid sequence of SEQ ID NO: 1 or SEQ ID NO: 2, wherein the engineered immune cell lacks expression of a cytokine, optionally wherein the nucleic acid sequence is any one of SEQ ID NOs: 7-9 or optionally wherein the cytokine is TNFα, and/or T-Cell-Specific Transcription Factor (TCF-1).

2. The engineered immune cell of claim 1, wherein the engineered immune cell is a T cell, a CD4+ T cell, a CD8+ T cell, a B cell, a natural killer (NK) cell, a natural killer T (NKT) cell, a dendritic cell, a myeloid cell, a monocyte, a macrophage, or a tumor-infiltrating immune cell or wherein the engineered immune cell is derived from an autologous donor or an allogenic donor.

3. The engineered immune cell of claim 1, wherein the non-endogenous expression vector including the GLUT5 nucleic acid sequence is a plasmid, a cosmid, a bacmid, a bacterial artificial chromosome (BAC), a yeast artificial chromosome (YAC), a viral vector, or a retroviral vector.

4. The engineered immune cell of claim 1, wherein the GLUT5 nucleic acid sequence is operably linked to an expression control sequence, optionally wherein the expression control sequence is an inducible promoter, a constitutive promoter, a native GLUT5 promoter, or a heterologous promoter.

5. (canceled)

6. (canceled)

7. The engineered immune cell of claim 1, further comprising a receptor that binds to a target antigen and/or a nucleic acid encoding the receptor,
optionally wherein
the receptor is a native cell receptor, a non-native cell receptor, a T-cell receptor, or a chimeric antigen receptor (CAR); or
the target antigen comprises a tumor antigen, optionally wherein the tumor antigen is 5T4, alpha 5β1-integrin, 707-AP, A33, AFP, ART-4, B7H4, BAGE, Bcl-2, β-catenin, BCMA, Bcr-abl, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC16, MUM-1-B, myc, MUM-2, MUM-3, NA88-A, NYESO-1, NY-Eso-B, p53, proteinase-3, p190 minor bcr-abl, Pml/RARα, PRAME, progesterone receptor, PSA, PSCA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, or WT1.

8. (canceled)

9. (canceled)

10. (canceled)

11. The engineered immune cell of claim 7, wherein the CAR comprises (i) an extracellular antigen binding domain; (ii) a transmembrane domain; and (iii) an intracellular domain, wherein the extracellular antigen binding domain binds to the target antigen,
optionally wherein
the extracellular antigen binding fragment is a single-chain variable fragment (scFv); or
the transmembrane domain comprises a CD8 transmembrane domain or a CD28 transmembrane domain; or
the intracellular domain comprises a CD3C signaling domain and optionally one or more costimulatory domains selected from a CD28 costimulatory domain, a 4-1BB costimulatory domain, an OX40 costimulatory domain, an ICOS costimulatory domain, a DAP-10 costimulatory domain, a PD-1 costimulatory domain, a CTLA-4 costimulatory domain, a LAG-3 costimulatory domain, a 2B4 costimulatory domain, a BTLA costimulatory domain, or any combination thereof.

12. (canceled)

13. (canceled)

14. (canceled)

15. (canceled)

16. A composition comprising an effective amount of the engineered immune cell of claim 1 and a pharmaceutically acceptable carrier.

17. (canceled)

18. (canceled)

19. (canceled)

20. (canceled)

21. (canceled)

22. (canceled)

23. (canceled)

24. (canceled)

25. (canceled)

26. A method for treating cancer or inhibiting tumor growth or metastasis or restoring T cell functionality under limiting glucose conditions in vivo in a subject in need thereof comprising administering to a recipient subject an effective amount of an engineered immune cell comprising a non-endogenous expression vector that includes a nucleic acid sequence encoding a Glucose Transporter 5 (GLUT5) amino acid sequence of SEQ ID NO: 1 or SEQ ID NO: 2, optionally wherein the nucleic acid sequence is any one of SEQ ID NOs: 7-9.

27. The method of claim 26, wherein the cancer or tumor is selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

28. The method of claim 26, wherein the engineered immune cell is administered pleurally, intravenously, subcutaneously, intranodally, intratumorally, intrathecally, intrapleurally or intraperitoneally.

29. The method of claim 26, further comprising sequentially, separately, or simultaneously administering to the subject an additional cancer therapy.

30. The method of claim 29, wherein the additional cancer therapy is selected from among chemotherapeutic agents, immune checkpoint inhibitors, monoclonal antibodies that specifically target tumor antigens, immune activating agents (e.g., interferons, interleukins, cytokines), oncolytic virus therapy and cancer vaccines.

31. The method of claim 26, further comprising sequentially, separately, or simultaneously administering to the subject one or more of: fructose; a pyruvate kinase M2 (PKM2) activator, DASA58, a ketohexokinase (KHK) inhibitor, or 6-(4-(2-Hydroxyethyl)piperazin-1-yl)-2-(3-(hydroxymethyl)-piperidin-1-yl)-4-(trifluoromethyl)nicotinonitrile.

32. A kit comprising an expression vector that includes a nucleic acid sequence encoding a Glucose Transporter 5 (GLUT5) amino acid sequence of SEQ ID NO: 1 or SEQ ID NO: 2 and one or more of: fructose, a pyruvate kinase M2 (PKM2) activator, a ketohexokinase (KHK) inhibitor, or an additional anti-cancer therapeutic agent, optionally wherein the nucleic acid sequence is any one of SEQ ID NOs: 7-9, and instructions for transducing immune cells with the expression vector.

33. The kit of claim 33 further comprising a vector encoding an engineered T-cell receptor (TCR) or other cell-surface ligand that binds to a target antigen.

34. (canceled)

35. (canceled)

36. The method of claim 26, wherein the engineered immune cell is obtained by isolating immune cells from a donor subject; and transducing the immune cells with the non-endogenous expression vector that includes the nucleic acid sequence encoding a Glucose Transporter 5 (GLUT5) amino acid sequence of SEQ ID NO: 1 or SEQ ID NO: 2.

37. The method of claim 36, wherein the donor subject and the recipient subject are the same or different.

38. The method of claim 36, wherein the immune cells isolated from the donor subject comprise one or more lymphocytes selected from among T cells, B cells, tumor infiltrating lymphocytes, or natural killer cells.

39. The method of claim 38, wherein the T cells are CD8⁺ cytotoxic T cells or CD4⁺ T cells, wherein the T cells comprise a native T cell receptor (TCR), a non-native TCR, or a chimeric antigen receptor (CAR).

40. The method of claim 39, wherein the chimeric antigen receptor (CAR) binds to a tumor antigen.
